SEQUENCE LISTING (85 sequences)

SEQ ID NO: 1 - DNA, 30 bp, Artificial Sequence
  Description of Artificial Sequence Synthetic oligonucleotide
ttttttgaattcgttaccctctctaacttc

SEQ ID NO: 2 - DNA, 33 bp, Artificial Sequence
  Description of Artificial Sequence Synthetic oligonucleotide
tttttttctagattacctccgactgcgtcttga

SEQ ID NO: 3 - DNA, 40 bp, Artificial Sequence
  Description of Artificial Sequence Synthetic oligonucleotide
gatctgcagcacgtgtctagaggatatcgaattcgcggcc

SEQ ID NO: 4 - DNA, 40 bp, Artificial Sequence
  Description of Artificial Sequence Synthetic oligonucleotide
gatccgcggccgcgaattcgatatcctctagacacgtgct

SEQ ID NO: 5 - DNA, 30 bp, Artificial Sequence
  Description of Artificial Sequence Synthetic oligonucleotide
ttttttgaattctcactgacagtgcagaca

SEQ ID NO: 6 - DNA, 33 bp, Artificial Sequence
  Description of Artificial Sequence Synthetic oligonucleotide
tttttttctagattagctgtaagctggggccac

SEQ ID NO: 7 - DNA, 30 bp, Artificial Sequence
  Description of Artificial Sequence Synthetic oligonucleotide
ttttttgaattcttcaactgccttggaatg

SEQ ID NO: 8 - DNA, 33 bp, Artificial Sequence
  Description of Artificial Sequence Synthetic oligonucleotide
tttttttctagattaagcgtgcacgttcacgga

SEQ ID NO: 9 - DNA, 36 bp, Artificial Sequence
  Description of Artificial Sequence Synthetic oligonucleotide
ttttttgatatcaccggaattgcagtcatgattggc

SEQ ID NO: 10 - DNA, 36 bp, Artificial Sequence
  Description of Artificial Sequence Synthetic oligonucleotide
ttttttgatatcatgtataatgctgatatgattgac

SEQ ID NO: 11 - DNA, 36 bp, Artificial Sequence
  Description of Artificial Sequence Synthetic oligonucleotide
tttttttctagattaacgttttcccgaggcgaagtc

SEQ ID NO: 12 - DNA, 42 bp, Artificial Sequence
  Description of Artificial Sequence Synthetic oligonucleotide
atcatcgagctccagctgtaattcatggtcgaaaagaagtgc

SEQ ID NO: 13 - DNA, 73 bp, Artificial Sequence
  Description of Artificial Sequence Synthetic oligonucleotide
gaattcctcgagctgcagcccgggtttttatagctaattagtcattttttgagagtaccacttcagctacctc

SEQ ID NO: 14 - DNA, 72 bp, Artificial Sequence
  Description of Artificial Sequence Synthetic oligonucleotide
cccgggctgcagctcgaggaattctttttattgattaactagtcattataaagatctaaaatgcataatttc

SEQ ID NO: 15 - DNA, 45 bp, Artificial Sequence
  Description of Artificial Sequence Synthetic oligonucleotide
gatgatggtaccgtaaacaaatataatgaaaagtattctaaacta

SEQ ID NO: 16 - DNA, 34 bp, Artificial Sequence
  Description of Artificial Sequence Synthetic oligonucleotide
aaacccgggttctttattctatacttaaaaagtg

SEQ ID NO: 17 - DNA, 43 bp, Artificial Sequence
  Description of Artificial Sequence Synthetic oligonucleotide
aaaagaattcgtcgactacgatacaaacttaacggatatcgcg

SEQ ID NO: 18 - DNA, 33 bp, Artificial Sequence
  Description of Artificial Sequence Synthetic oligonucleotide
ttttcgcgaaccggaattgcagtcatgattggc

SEQ ID NO: 19 - DNA, 39 bp, Artificial Sequence
  Description of Artificial Sequence Synthetic oligonucleotide
ttttgtcgacgcggccgcttaagcgtgcacgttcacgga

SEQ ID NO: 20 - DNA, 42 bp, Artificial Sequence
  Description of Artificial Sequence Synthetic oligonucleotide
atcatcgagctcgcggccgcctatcaaaagtcttaatgagtt

SEQ ID NO: 21 - DNA, 73 bp, Artificial Sequence
  Description of Artificial Sequence Synthetic oligonucleotide
gaattcctcgagctgcagcccgggtttttatagctaattagtcattttttcgtaagtaagtatttttatttaa

SEQ ID NO: 22 - DNA, 72 bp, Artificial Sequence
  Description of Artificial Sequence Synthetic oligonucleotide
cccgggctgcagctcgaggaattctttttattgattaactagtcaaatgagtatatataattgaaaaagtaa

SEQ ID NO: 23 - DNA, 45 bp, Artificial Sequence
  Description of Artificial Sequence Synthetic oligonucleotide
gatgatggtaccttcataaatacaagtttgattaaacttaagttg

SEQ ID NO: 24 - DNA, 33 bp, Artificial Sequence
  Description of Artificial Sequence Synthetic oligonucleotide
aaacccgggcggtggtttgcgattccgaaatct

SEQ ID NO: 25 - DNA, 43 bp, Artificial Sequence
  Description of Artificial Sequence Synthetic oligonucleotide
aaaagaattcggatccgattaaacctaaataattgtactttgt

SEQ ID NO: 26 - DNA, 33 bp, Artificial Sequence
  Description of Artificial Sequence Synthetic oligonucleotide
tttcacgtgatgtataatgctgatatgattgac

SEQ ID NO: 27 - DNA, 42 bp, Artificial Sequence
  Description of Artificial Sequence Synthetic oligonucleotide
ttttggatccgcggccgcttaacgttttcccgaggcgaagtc

SEQ ID NO: 28 - DNA, 39 bp, Artificial Sequence
  Description of Artificial Sequence Synthetic oligonucleotide
ttttttgatatcatgaccggaattgcagtcatgattggc

SEQ ID NO: 29 - DNA, 36 bp, Artificial Sequence
  Description of Artificial Sequence Synthetic oligonucleotide
ttttcgcgaatgaccggaattgcagtcatgattggc

SEQ ID NO: 30 - DNA, 42 bp, Artificial Sequence
  Description of Artificial Sequence Synthetic oligonucleotide
atcatcgagctcgaccctttacaagaataaaagaagaaacaa

SEQ ID NO: 31 - DNA, 73 bp, Artificial Sequence
  Description of Artificial Sequence Synthetic oligonucleotide
ctcgagctgcaggaattccccgggtttttattagctaattagcaatatagattcaatatgataattactctaa

SEQ ID NO: 32 - DNA, 72 bp, Artificial Sequence
  Description of Artificial Sequence Synthetic oligonucleotide
cccggggaattcctgcagctcgagtttttattgactagttaatcataagataaataatatacagcattgtaa

SEQ ID NO: 33 - DNA, 45 bp, Artificial Sequence
  Description of Artificial Sequence Synthetic oligonucleotide
gatgatggtaccgggtaatggcttttgtttataaccacgtttgtc

SEQ ID NO: 34 - DNA, 95 bp, Artificial Sequence
  Description of Artificial Sequence Synthetic oligonucleotide
aaacccgggttaattaattagtcatcaggcagggcgaaacgagactatctgctcgttaattaattagagcttctttattctatacttaaaaagtg

SEQ ID NO: 35 - DNA, 43 bp, Artificial Sequence
  Description of Artificial Sequence Synthetic oligonucleotide
aaaactgcaggtcgactacgatacaaacttaacggatatcgcg

SEQ ID NO: 36 - DNA, 58 bp, Artificial Sequence
  Description of Artificial Sequence Synthetic oligonucleotide
ttttcgcgatatccgttaagtttgtatcgtaatgaccggaattgcagtcatgattggc

SEQ ID NO: 37 - DNA, 43 bp, Artificial Sequence
  Description of Artificial Sequence Synthetic oligonucleotide
ttttgtcgactctagataaaaattaagcgtgcacgttcacgga

SEQ ID NO: 38 - DNA, 30 bp, Artificial Sequence
  Description of Artificial Sequence Synthetic primer
gaagtggccatcttcgtgcacggaccaact

SEQ ID NO: 39 - DNA, 30 bp, Artificial Sequence
  Description of Artificial Sequence Synthetic primer
agttggtccgtgcacgaagatggccacttc

SEQ ID NO: 40 - DNA, 30 bp, West Nile Virus
  CDS (1)..(30)
gaagtggccatttttgtccatggaccaact

SEQ ID NO: 41 - protein, 10 aa, West Nile Virus
EVAIFVHGPT

SEQ ID NO: 42 - DNA, 24 bp, Artificial Sequence
  Description of Artificial Sequence Synthetic primer
aagcctaccatcgatgtgaagatg

SEQ ID NO: 43 - DNA, 51 bp, Artificial Sequence
  Description of Artificial Sequence Synthetic primer
gggccctctagataaaaattaagcgtgcacgttcacggagaggaagagcag

SEQ ID NO: 44 - DNA, 51 bp, Artificial Sequence
  Description of Artificial Sequence Synthetic primer
ctgctcttcctctccgtgaacgtgcacgcttaatttttatctagagggccc

SEQ ID NO: 45 - protein, 10 aa, Artificial Sequence
  Description of Artificial Sequence Synthetic peptide
LLFLSVNVHA

SEQ ID NO: 46 - protein, 8 aa, Artificial Sequence
  Description of Artificial Sequence Synthetic peptide
KPTIDVKM

SEQ ID NO: 47 - DNA, 59 bp, Artificial Sequence
  Description of Artificial Sequence Synthetic primer
atcgcgatatccgttaagtttgtatcgtaatgaccggaattgcagtcatgattggcctg

SEQ ID NO: 48 - DNA, 24 bp, Artificial Sequence
  Description of Artificial Sequence Synthetic primer
catcttcacatcgatggtaggctt

SEQ ID NO: 49 - DNA, 24 bp, Artificial Sequence
  Description of Artificial Sequence Synthetic primer
aagcctaccatcgatgtgaagatg

SEQ ID NO: 50 - protein, 8 aa, Artificial Sequence
  Description of Artificial Sequence Synthetic peptide
KPTIDVKM

SEQ ID NO: 51 - protein, 10 aa, Artificial Sequence
  Description of Artificial Sequence Synthetic peptide
MTGIAVMIGL

SEQ ID NO: 52 - DNA, 41 bp, Artificial Sequence
  Description of Artificial Sequence Synthetic oligonucleotide
cgatatccgttaagtttgtatcgtaatgcccgggtcgcgaa

SEQ ID NO: 53 - DNA, 45 bp, Artificial Sequence
  Description of Artificial Sequence Synthetic oligonucleotide
agctttcgcgacccgggcattacgatacaaacttaacggatatcg

SEQ ID NO: 54 - DNA, 4270 bp, West Nile virus
  CDS (1760)..(3811)
tgaatgttaaatgttatactttggatgaagctataaatatgcattggaaaaataatccatttaaagaaaggattcaaatactacaaaacctaagcgataatatgttaactaagcttattcttaacgacgctttaaatatacacaaataaacataatttttgtataacctaacaaataactaaaacataaaaataataaaaggaaatgtaatatcgtaattattttactcaggaatggggttaaatatttatatcacgtgtatatctatactgttatcgtatactctttacaattactattacgaatatgcaagagataataagattacgtatttaagagaatcttgtcatgataattgggtacgacatagtgataaatgctatttcgcatcgttacataaagtcagttggaaagatggatttgacagatgtaacttaataggtgcaaaaatgttaaataacagcattctatcggaagataggataccagttatattatacaaaaatcactggttggataaaacagattctgcaatattcgtaaaagatgaagattactgcgaatttgtaaactatgacaataaaaagccatttatctcaacgacatcgtgtaattcttccatgttttatgtatgtgtttcagatattatgagattactataaactttttgtatacttatattccgtaaactatattaatcatgaagaaaatgaaaaagtatagaagctgttcacgagcggttgttgaaaacaacaaaattatacattcaagatggcttacatatacgtctgtgaggctatcatggataatgacaatgcatctctaaataggtttttggacaatggattcgaccctaacacggaatatggtactctacaatctcctcttgaaatggctgtaatgttcaagaataccgaggctataaaaatcttgatgaggtatggagctaaacctgtagttactgaatgcacaacttcttgtctgcatgatgcggtgttgagagacgactacaaaatagtgaaagatctgttgaagaataactatgtaaacaatgttctttacagcggaggctttactcctttgtgtttggcagcttaccttaacaaagttaatttggttaaacttctattggctcattcggcggatgtagatatttcaaacacggatcggttaactcctctacatatagccgtatcaaataaaaatttaacaatggttaaacttctattgaacaaaggtgctgatactgacttgctggataacatgggacgtactcctttaatgatcgctgtacaatctggaaatattgaaatatgtagcacactacttaaaaaaaataaaatgtccagaactgggaaaaattgatcttgccagctgtaattcatggtagaaaagaagtgctcaggctacttttcaacaaaggagcagatgtaaactacatctttgaaagaaatggaaaatcatatactgttttggaattgattaaagaaagttactctgagacacaaaagaggtagctgaagtggtactctcaaaaaggtacgtgactaattagctataaaaaggatccgggttaattaattagtcatcaggcagggcgagaacgagactatctgctcgttaattaattagagcttctttattctatacttaaaaagtgaaaataaatacaaaggttcttgagggttgtgttaaattgaaagcgagaaataatcataaattatttcattatcgcgatatccgttaagtttgtatcgtaatgaccggaattgcagtcatgattggcctgatcgccagcgtaggagcagttaccctctctaacttccaagggaaggtgatgatgacggtaaatgctactgacgtcacagatgtcatcacgattccaacagctgctggaaagaacctatgcattgtcagagcaatggatgtgggatacatgtgcgatgatactatcacttatgaatgcccagtgctgtcggctggtaatgatccagaagacatcgactgttggtgcacaaagtcagcagtctacgtcaggtatggaagatgcaccaagacacgccactcaagacgcagtcggaggtcactgacagtgcagacacacggagaaagcactctagcgaacaagaagggggcttggatggacagcaccaaggccacaaggtatttggtaaaaacagaatcatggatcttgaggaaccctggatatgccctggtggcagccgtcattggttggatgcttgggagcaacaccatgcagagagttgtgtttgtcgtgctattgcttttggtggccccagcttacagcttcaactgccttggaatgagcaacagagacttcttggaaggagtgtctggagcaacatgggtggatttggttctcgaaggcgacagctgcgtgactatcatgtctaaggacaagcctaccatcgatgtgaagatgatgaatatggaggcggccaacctggcagaggtccgcagttattgctatttggctaccgtcagcgatctctccaccaaagctgcgtgcccgaccatgggagaagctcacaatgacaaacgtgctgacccagcttttgtgtgcagacaaggagtggtggacaggggctggggcaacggctgcggactatttggcaaaggaagcattgacacatgcgccaaatttgcctgctctaccaaggcaataggaagaaccatcttgaaagagaatatcaagtacgaagtggccatcttcgtgcacggaccaactactgtggagtcgcacggaaactactccacacaggttggagccactcaggcagggagattcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagcggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccattggcacaagtctggaagcagcattggcaaagcctttacaaccaccctcaaaggagcgcagagactagccgctctaggagacacagcttgggactttggatcagttggaggggtgttcacctcagttgggaaggctgtccatcaagtgttcggaggagcattccgctcactgttcggaggcatgtcctggataacgcaaggattgctgggggctctcctgttgtggatgggcatcaatgctcgtgataggtccatagctctcacgtttctcgcagttggaggagttctgctcttcctctccgtgaacgtgcacgcttaatttttatctagaatcgatcccgggtttttatgactagttaatcacggccgccttataaagatctaaaatgcataatttctaaataatgaaaaaaagtacatcatgagcaacgcgttagtatattttacaatggagattaacgctctataccgttctatgtttattgattcagatgatgttttagaaaagaaagttattgaatatgaaaactttaatgaagatgaagatgacgacgatgattattgttgtaaatctgttttagatgaagaagatgacgcgctaaagtatactatggttacaaagtataagtctatactactaatggcgacttgtgcaagaaggtatagtatagtgaaaatgttgttagattatgattatgaaaaaccaaataaatcagatccatatctaaaggtatctcctttgcacataatttcatctattcctagtttagaatac

SEQ ID NO: 55 - protein, 684 aa, West Nile virus
MTGIAVMIGLIASVGAVTLSNFQGKVMMTVNATDVTDVITIPTAAGKNLCIVRAMDVGYMCDDTITYECPVLSAGNDPEDIDCWCTKSAVYVRYGRCTKTRHSRRSRRSLTVQTHGESTLANKKGAWMDSTKATRYLVKTESWILRNPGYALVAAVIGWMLGSNTMQRVVFVVLLLLVAPAYSFNCLGMSNRDFLEGVSGATWVDLVLEGDSCVTIMSKDKPTIDVKMMNMEAANLAEVRSYCYLATVSDLSTKAACPTMGEAHNDKRADPAFVCRQGVVDRGWGNGCGLFGKGSIDTCAKFACSTKAIGRTILKENIKYEVAIFVHGPTTVESHGNYSTQVGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSAASLNDLTPVGRLVTVNPFVSVATANAKVLIELEPPFGDSYIVVGRGEQQINHHWHKSGSSIGKAFTTTLKGAQRLAALGDTAWDFGSVGGVFTSVGKAVHQVFGGAFRSLFGGMSWITQGLLGALLLWMGINARDRSIALTFLAVGGVLLFLSVNVHA

SEQ ID NO: 56 - DNA, 5109 bp, West Nile virus
  CDS (1640)..(3691)
gaccctttacaagaataaaagaagaaacaactgtgaaatagtttataaatgtaattcgtatgcagaaaacgataatatattttggtatgagaaatctaaaggagacatagtttgtatagacatgcgctcttccgatgagatattcgatgcttttctaatgtatcatatagctacaagatatgcctatcatgatgatgatatatatctacaaatagtgttatattattctaataatcaaaatgttatatcttatattacgaaaaataaatacgttaagtatataagaaataaaactagagacgatattcataaagtaaaaatattagctctagaagactttacaacggaagaaatatattgttggattagtaatatataacagcgtagctgcacggttttgatcattttccaacaatataaaccaatgaaggaggacgactcatcaaacataaataacattcacggaaaatattcagtatcagatttatcacaagatgattatgttattgaatgtatagacggatcttttgattcgatcaagtatagagatataaaggttataataatgaagaataacggttacgttaattgtagtaaattatgtaaaatgcggaataaatacttttctagatggttgcgtctttctacttctaaagcattattagacatttacaataataagtcagtagataatgctattgttaaagtctatggtaaaggtaagaaacttattataacaggattttatctcaaacaaaatatgatacgttatgttattgagtggataggggatgattttacaaacgatatatacaaaatgattaatttctataatgcgttattcggtaacgatgaattaaaaatagtatcctgtgaaaacactctatgcccgtttatagaacttggtagatgctattatggtaaaaaatgtaagtatatacacggagatcaatgtgatatctgtggtctatatatactacaccctaccgatattaaccaacgagtttctcacaagaaaacttgtttagtagatagagattctttgattgtgtttaaaagaagtaccagtaaaaagtgtggcatatgcatagaagaaataaacaaaaaacatatttccgaacagtattttggaattctcccaagttgtaaacatattttttgcctatcatgtataagacgttgggcagatactaccagaaatacagatactgaaaatacgtgtcctgaatgtagaatagtttttcctttcataatacccagtaggtattggatagataataaatatgataaaaaaatattatataatagatataagaaaatgatttttacaaaaatacctataagaacaataaaaatataattacatttacggaaaatagctggttttagtttaccaacttagagtaattatcatattgaatctatattgctaattagctaataaaaacccgggttaattaattagtcatcaggcagggcgagaacgagactatctgctcgttaattaattagagcttctttattctatacttaaaaagtgaaaataaatacaaaggttcttgagggttgtgttaaattgaaagcgagaaataatcataaattatttcattatcgcgatatccgttaagtttgtatcgtaatgaccggaattgcagtcatgattggcctgatcgccagcgtaggagcggttaccctctctaacttccaagggaaggtgatgatgacggtaaatgctactgacgtcacagatgtcatcacgattccaacagctgctggaaagaacctatgcattgtcagagcaatggatgtgggatacatgtgcgatgatactatcacttatgaatgcccagtgctgtcggctggtaatgatccagaagacatcgactgttggtgcacaaagtcagcagtctacgtcaggtatggaagatgcaccaagacacgccactcaagacgcagtcggaggtcactgacagtgcagacacacggagaaagcactctagcgaacaagaagggggcttggatggacagcaccaaggccacaaggtatttggtaaaaacagaatcatggatcttgaggaaccctggatatgccctggtggcagccgtcattggttggatgcttgggagcaacaccatgcagagagttgtgtttgtcgtgctattgcttttggtggccccagcttacagcttcaactgccttggaatgagcaacagagacttcttggaaggagtgtctggagcaacatgggtggatttggttctcgaaggcgacagctgcgtgactatcatgtctaaggacaagcctaccatcgatgtgaagatgatgaatatggaggcggccaacctggcagaggtccgcagttattgctatttggctaccgtcagcgatctctccaccaaagctgcgtgcccgaccatgggagaagctcacaatgacaaacgtgctgacccagcttttgtgtgcagacaaggagtggtggacaggggctggggcaacggctgcggactatttggcaaaggaagcattgacacatgcgccaaatttgcctgctctaccaaggcaataggaagaaccatcttgaaagagaatatcaagtacgaagtggccatcttcgtgcacggaccaactactgtggagtcgcacggaaactactccacacaggttggagccactcaggcagggagattcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagcggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccattggcacaagtctggaagcagcattggcaaagcctttacaaccaccctcaaaggagcgcagagactagccgctctaggagacacagcttgggactttggatcagttggaggggtgttcacctcagttgggaaggctgtccatcaagtgttcggaggagcattccgctcactgttcggaggcatgtcctggataacgcaaggattgctgggggctctcctgttgtggatgggcatcaatgctcgtgataggtccatagctctcacgtttctcgcagttggaggagttctgctcttcctctccgtgaacgtgcacgcttaatttttatctagagtcgagtttttattgactagttaatcataagataaataatatacagcattgtaaccatcgtcatccgttatacggggaataatattaccatacagtattattaaattttcttacgaagaatatagatcggtatttatcgttagtttattttacatttattaattaaacatgtctactattacctgttatggaaatgacaaatttagttatataatttatgataaaattaagataataataatgaaatcaaataattatgtaaatgctactagattatgtgaattacgaggaagaaagtttacgaactggaaaaaattaagtgaatctaaaatattagtcgataatgtaaaaaaaataaatgataaaactaaccagttaaaaacggatatgattatatacgttaaggatattgatcataaaggaagagatacttgcggttactatgtacaccaagatctggtatcttctatatcaaattggatatctccgttattcgccgttaaggtaaataaaattattaactattatatatgtaatgaatatgatatacgacttagcgaaatggaatctgatatgacagaagtaatagatgtagttgataaattagtaggaggatacaatgatgaaatagcagaaataatatatttgtttaataaatttatagaaaaatatattgctaacatatcgttatcaactgaattatctagtatattaaataattttataaattttaataaaaaatacaataacgacataaaagatattaaatctttaattcttgatctgaaaaacacatctataaaactagataaaaagttattcgataaagataataatgaatcgaacgatgaaaaattggaaacagaagttgataagctaatttttttcatctaaatagtattattttattgaagtacgaagttttacgttagataaataataaaggtcgatttttactttgttaaatatcaaatatgtcattatctgataaagatacaaaaacacacggtgattatcaaccatctaacgaacagatattacaaaaaatacgtcggactatggaaaacgaagctgatagcctcaatagaagaagcattaaagaaattgttgtagatgttatgaagaattgggatcatcctctcaacgaagaaatagataaagttctaaactggaaaaatgatacattaaacgatttagatcatctaaatacagatgataatattaaggaaatcatacaatgtctgattagagaatttgcgtttaaaaagatcaattctattatgtatagttatgctatggtaaaactcaattcagataacgaaacattgaaagataaaattaaggattattttatagaaactattcttaaagacaaacgtggttataaacaaaagccattaccc

SEQ ID NO: 57 - protein, 684 aa, West Nile virus; identical to SEQ ID NO: 55

SEQ ID NO: 58 - DNA, 2087 bp, West Nile virus
  CDS (15)..(2072)
ctgcagccgccaccatgggatcaaccggaattgcagtcatgattggcctgatcgccagcgtaggagcagttaccctctctaacttccaagggaaggtgatgatgacggtaaatgctactgacgtcacagatgtcatcacgattccaacagctgctggaaagaacctatgcattgtcagagcaatggatgtgggatacatgtgcgatgatactatcacttatgaatgcccagtgctgtcggctggtaatgatccagaagacatcgactgttggtgcacaaagtcagcagtctacgtcaggtatggaagatgcaccaagacacgccactcaagacgcagtcggaggtcactgacagtgcagacacacggagaaagcactctagcgaacaagaagggggcttggatggacagcaccaaggccacaaggtatttggtaaaaacagaatcatggatcttgaggaaccctggatatgccctggtggcagccgtcattggttggatgcttgggagcaacaccatgcagagagttgtgtttgtcgtgctattgcttttggtggccccagcttacagcttcaactgccttggaatgagcaacagagacttcttggaaggagtgtctggagcaacatgggtggatttggttctcgaaggcgacagctgcgtgactatcatgtctaaggacaagcctaccatcgatgtgaagatgatgaatatggaggcggccaacctggcagaggtccgcagttattgctatttggctaccgtcagcgatctctccaccaaagctgcgtgcccgaccatgggagaagctcacaatgacaaacgtgctgacccagcttttgtgtgcagacaaggagtggtggacaggggctggggcaacggctgcggactatttggcaaaggaagcattgacacatgcgccaaatttgcctgctctaccaaggcaataggaagaaccatcttgaaagagaatatcaagtacgaagtggccatttttgtccatggaccaactactgtggagtcgcacggaaactactccacacaggttggagccactcaggcagggagattcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagcggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccattggcacaagtctggaagcagcattggcaaagcctttacaaccaccctcaaaggagcgcagagactagccgctctaggagacacagcttgggactttggatcagttggaggggtgttcacctcagttgggaaggctgtccatcaagtgttcggaggagcattccgctcactgttcggaggcatgtcctggataacgcaaggattgctgggggctctcctgttgtggatgggcatcaatgctcgtgataggtccatagctctcacgtttctcgcagttggaggagttctgctcttcctctccgtgaacgtgcacgcttaatttttatctaga

SEQ ID NO: 59 - protein, 686 aa, West Nile virus
MGSTGIAVMIGLIASVGAVTLSNFQGKVMMTVNATDVTDVITIPTAAGKNLCIVRAMDVGYMCDDTITYECPVLSAGNDPEDIDCWCTKSAVYVRYGRCTKTRHSRRSRRSLTVQTHGESTLANKKGAWMDSTKATRYLVKTESWILRNPGYALVAAVIGWMLGSNTMQRVVFVVLLLLVAPAYSFNCLGMSNRDFLEGVSGATWVDLVLEGDSCVTIMSKDKPTIDVKMMNMEAANLAEVRSYCYLATVSDLSTKAACPTMGEAHNDKRADPAFVCRQGVVDRGWGNGCGLFGKGSIDTCAKFACSTKAIGRTILKENIKYEVAIFVHGPTTVESHGNYSTQVGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSAASLNDLTPVGRLVTVNPFVSVATANAKVLIELEPPFGDSYIVVGRGEQQINHHWHKSGSSIGKAFTTTLKGAQRLAALGDTAWDFGSVGGVFTSVGKAVHQVFGGAFRSLFGGMSWITQGLLGALLLWMGINARDRSIALTFLAVGGVLLFLSVNVHA

SEQ ID NO: 60 - DNA, 6928 bp, West Nile virus
  CDS (2005)..(4056)
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcggccgcaattctgaatgttaaatgttatactttggatgaagctataaatatgcattggaaaaataatccatttaaagaaaggattcaaatactacaaaacctaagcgataatatgttaactaagcttattcttaacgacgctttaaatatacacaaataaacataatttttgtataacctaacaaataactaaaacataaaaataataaaaggaaatgtaatatcgtaattattttactcaggaatggggttaaatatttatatcacgtgtatatctatactgttatcgtatactctttacaattactattacgaatatgcaagagataataagattacgtatttaagagaatcttgtcatgataattgggtacgacatagtgataaatgctatttcgcatcgttacataaagtcagttggaaagatggatttgacagatgtaacttaataggtgcaaaaatgttaaataacagcattctatcggaagataggataccagttatattatacaaaaatcactggttggataaaacagattctgcaatattcgtaaaagatgaagattactgcgaatttgtaaactatgacaataaaaagccatttatctcaacgacatcgtgtaattcttccatgttttatgtatgtgtttcagatattatgagattactataaactttttgtatacttatattccgtaaactatattaatcatgaagaaaatgaaaaagtatagaagctgttcacgagcggttgttgaaaacaacaaaattatacattcaagatggcttacatatacgtctgtgaggctatcatggataatgacaatgcatctctaaataggtttttggacaatggattcgaccctaacacggaatatggtactctacaatctcctcttgaaatggctgtaatgttcaagaataccgaggctataaaaatcttgatgaggtatggagctaaacctgtagttactgaatgcacaacttcttgtctgcatgatgcggtgttgagagacgactacaaaatagtgaaagatctgttgaagaataactatgtaaacaatgttctttacagcggaggctttactcctttgtgtttggcagcttaccttaacaaagttaatttggttaaacttctattggctcattcggcggatgtagatatttcaaacacggatcggttaactcctctacatatagccgtatcaaataaaaatttaacaatggttaaacttctattgaacaaaggtgctgatactgacttgctggataacatgggatgtactcctttaatgatcgctgtacaatctggaaatattgaaatatgtagcacactacttaaaaaaaataaaatgtccagaactgggaaaaattgatcttgccagctgtaattcatggtagaaaagaagtgctcaggctacttttcaacaaaggagcagatgtaaactacatctttgaaagaaatggaaaatcatatactgttttggaattgattaaagaaagttactctgagacacaaaagaggtagctgaagtggtactctcaaaggtacgtgactaattagctataaaaaggatccgggttaattaattagtcatcaggcagggcgagaacgagactatctgctcgttaattaattagagcttctttattctatacttaaaaagtgaaaataaatacaaaggttcttgagggttgtgttaaattgaaagcgagaaataatcataaattatttcattatcgcgatatccgttaagtttgtatcgtaatgaccggaattgcagtcatgattggcctgatcgccagcgtaggagcggttaccctctctaacttccaagggaaggtgatgatgacggtaaatgctactgacgtcacagatgtcatcacgattccaacagctgctggaaagaacctatgcattgtcagagcaatggatgtgggatacatgtgcgatgatactatcacttatgaatgcccagtgctgtcggctggtaatgatccagaagacatcgactgttggtgcacaaagtcagcagtctacgtcaggtatggaagatgcaccaagacacgccactcaagacgcagtcggaggtcactgacagtgcagacacacggagaaagcactctagcgaacaagaagggggcttggatggacagcaccaaggccacaaggtatttggtaaaaacagaatcatggatcttgaggaaccctggatatgccctggtggcagccgtcattggttggatgcttgggagcaacaccatgcagagagttgtgtttgtcgtgctattgcttttggtggccccagcttacagcttcaactgccttggaatgagcaacagagacttcttggaaggagtgtctggagcaacatgggtggatttggttctcgaaggcgacagctgcgtgactatcatgtctaaggacaagcctaccatcgatgtgaagatgatgaatatggaggcggccaacctggcagaggtccgcagttattgctatttggctaccgtcagcgatctctccaccaaagctgcgtgcccgaccatgggagaagctcacaatgacaaacgtgctgacccagcttttgtgtgcagacaaggagtggtggacaggggctggggcaacggctgcggactatttggcaaaggaagcattgacacatgcgccaaatttgcctgctctaccaaggcaataggaagaaccatcttgaaagagaatatcaagtacgaagtggccatcttcgtgcacggaccaactactgtggagtcgcacggaaactactccacacaggttggagccactcaggcagggagattcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagcggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccattggcacaagtctggaagcagcattggcaaagcctttacaaccaccctcaaaggagcgcagagactagccgctctaggagacacagcttgggactttggatcagttggaggggtgttcacctcagttgggaaggctgtccatcaagtgttcggaggagcattccgctcactgttcggaggcatgtcctggataacgcaaggattgctgggggctctcctgttgtggatgggcatcaatgctcgtgataggtccatagctctcacgtttctcgcagttggaggagttctgctcttcctctccgtgaacgtgcacgcttaatttttatctagaatcgatcccgggtttttatgactagttaatcacggccgcttataaagatctaaaatgcataatttctaaataatgaaaaaaagtacatcatgagcaacgcgttagtatattttacaatggagattaacgctctataccgttctatgtttattgattcagatgatgttttagaaaagaaagttattgaatatgaaaactttaatgaagatgaagatgacgacgatgattattgttgtaaatctgttttagatgaagaagatgacgcgctaaagtatactatggttacaaagtataagtctatactactaatggcgacttgtgcaagaaggtatagtatagtgaaaatgttgttagattatgattatgaaaaaccaaataaatcagatccatatctaaaggtatctcctttgcacataatttcatctattcctagtttagaatacctgcagccaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 61 - protein, 684 aa, West Nile virus; identical to SEQ ID NO: 55

SEQ ID NO: 62 - DNA, 37 bp, Artificial Sequence
  Description of Artificial Sequence Synthetic primer
attatcgcgaaccggaattgcagtcatgattggcctg

SEQ ID NO: 63 - protein, 9 aa, Artificial Sequence
  Description of Artificial Sequence Synthetic peptide
TGIAVMIGL

SEQ ID NO: 64 - DNA, 40 bp, Artificial Sequence
  Description of Artificial Sequence Synthetic primer
attatcgcgaatgaccggaattgcagtcatgattggcctg

SEQ ID NO: 65 - protein, 10 aa, Artificial Sequence
  Description of Artificial Sequence Synthetic peptide
MTGIAVMIGL

SEQ ID NO: 66 - DNA, 11029 bp, West Nile virus
  CDS (97)..(10395)
agtagttcgcctgtgtgagctgacaaacttagtagtgtttgtgaggattaacaacaattaacacagtgcgagctgtttcttagcacgaagatctcgatgtctaagaaaccaggagggcccggcaagagccgggctgtcaatatgctaaaacgcggaatgccccgcgtgttgtccttgattggactgaagagggctatgttgagcctgatcgacggcaaggggccaatacgatttgtgttggctctcttggcgttcttcaggttcacagcaattgctccgacccgagcagtgctggatcgatggagaggtgtgaacaaacaaacagcgatgaaacaccttctgagttttaagaaggaactagggaccttgaccagtgctatcaatcggcggagctcaaaacaaaagaaaagaggaggaaagaccggaattgcagtcatgattggcctgatcgccagcgtaggagcagttaccctctctaacttccaagggaaggtgatgatgacggtaaatgctactgacgtcacagatgtcatcacgattccaacagctgctggaaagaacctatgcattgtcagagcaatggatgtgggatacatgtgcgatgatactatcacttatgaatgcccagtgctgtcggctggtaatgatccagaagacatcgactgttggtgcacaaagtcagcagtctacgtcaggtatggaagatgcaccaagacacgccactcaagacgcagtcggaggtcactgacagtgcagacacacggagaaagcactctagcgaacaagaagggggcttggatggacagcaccaaggccacaaggtatttggtaaaaacagaatcatggatcttgaggaaccctggatatgccctggtggcagccgtcattggttggatgcttgggagcaacaccatgcagagagttgtgtttgtcgtgctattgcttttggtggccccagcttacagcttcaactgccttggaatgagcaacagagacttcttggaaggagtgtctggagcaacatgggtggatttggttctcgaaggcgacagctgcgtgactatcatgtctaaggacaagcctaccatcgatgtgaagatgatgaatatggaggcggccaacctggcagaggtccgcagttattgctatttggctaccgtcagcgatctctccaccaaagctgcgtgcccgaccatgggagaagctcacaatgacaaacgtgctgacccagcttttgtgtgcagacaaggagtggtggacaggggctggggcaacggctgcggactatttggcaaaggaagcattgacacatgcgccaaatttgcctgctctaccaaggcaataggaagaaccatcttgaaagagaatatcaagtacgaagtggccatttttgtccatggaccaactactgtggagtcgcacggaaactactccacacaggttggagccactcaggcagggagattcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagtggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccattggcacaagtctggaagcagcattggcaaagcctttacaaccaccctcaaaggagcgcagagactagccgctctaggagacacagcttgggactttggatcagttggaggggtgttcacctcagttgggaaggctgtccatcaagtgttcggaggagcattccgctcactgttcggaggcatgtcctggataacgcaaggattgctgggggctctcctgttgtggatgggcatcaatgctcgtgataggtccatagctctcacgtttctcgcagttggaggagttctgctcttcctctccgtgaacgtgcacgctgacactgggtgtgccatagacatcagccggcaagagctgagatgtggaagtggagtgttcatacacaatgatgtggaggcttggatggaccggtacaagtattaccctgaaacgccacaaggcctagccaagatcattcagaaagctcataaggaaggagtgtgcggtctacgatcagtttccagactggagcatcaaatgtgggaagcagtgaaggacgagctgaacactcttttgaaggagaatggtgtggaccttagtgtcgtggttgagaaacaggagggaatgtacaagtcagcacctaaacgcctcaccgccaccacggaaaaattggaaattggctggaaggcctggggaaagagtattttatttgcaccagaactcgccaacaacacctttgtggttgatggtccggagaccaaggaatgtccgactcagaatcgcgcttggaatagcttagaagtggaggattttggatttggtctcaccagcactcggatgttcctgaaggtcagagagagcaacacaactgaatgtgactcgaagatcattggaacggctgtcaagaacaacttggcgatccacagtgacctgtcctattggattgaaagcaggctcaatgatacgtggaagcttgaaagggcagttctgggtgaagtcaaatcatgtacgtggcctgagacgcataccttgtggggcgatggaatccttgagagtgacttgataataccagtcacactggcgggaccacgaagcaatcacaatcggagacctgggtacaagacacaaaaccagggcccatgggacgaaggccgggtagagattgacttcgattactgcccaggaactacggtcaccctgagtgagagctgcggacaccgtggacctgccactcgcaccaccacagagagcggaaagttgataacagattggtgctgcaggagctgcaccttaccaccactgcgctaccaaactgacagcggctgttggtatggtatggagatcagaccacagagacatgatgaaaagaccctcgtgcagtcacaagtgaatgcttataatgctgatatgattgacccttttcagttgggccttctggtcgtgttcttggccacccaggaggtccttcgcaagaggtggacagccaagatcagcatgccagctatactgattgctctgctagtcctggtgtttgggggcattacttacactgatgtgttacgctatgtcatcttggtgggggcagctttcgcagaatctaattcgggaggagacgtggtacacttggcgctcatggcgaccttcaagatacaaccagtgtttatggtggcatcgtttctcaaagcgagatggaccaaccaggagaacattttgttgatgttggcggctgttttctttcaaatggcttatcacgatgcccgccaaattctgctctgggagatccctgatgtgttgaattcactggcggtagcttggatgatactgagagccataacattcacaacgacatcaaacgtggttgttccgctgctagccctgctaacacccgggctgagatgcttgaatctggatgtgtacaggatactgctgttgatggtcggaataggcagcttgatcagggagaagaggagtgcagctgcaaaaaagaaaggagcaagtctgctatgcttggctctagcctcaacaggacttttcaaccccatgatccttgctgctggactgattgcatgtgatcccaaccgtaaacgcggatggcccgcaactgaagtgatgacagctgtcggcctaatgtttgccatcgtcggagggctggcagagcttgacattgactccatggccattccaatgactatcgcggggctcatgtttgctgctttcgtgatttctgggaaatcaacagatatgtggattgagagaacggcggacatttcctgggaaagtgatgcagaaattacaggctcgagcgaaagagttgatgtgcggcttgatgatgatggaaacttccagctcatgaatgatccaggagcaccttggaagatatggatgctcagaatggtctgtctcgcgattagtgcgtacaccccctgggcaatcttgccctcagtagttggattttggataactctccaatacacaaagagaggaggcgtgttgtgggacactccctcaccaaaggagtacaaaaagggggacacgaccaccggcgtctacaggatcatgactcgtgggctgctcggcagttatcaagcaggagcgggcgtgatggttgaaggtgttttccacaccctttggcatacaacaaaaggagccgctttgatgagcggagagggccgcctggacccatactggggcagtgtcaaggaggatcgactttgttacggaggaccctggaaattgcagcacaagtggaacgggcaggatgaggtgcagatgattgtggtggaacctggcaagaacgttaagaacgtccagacgaaaccaggggtgttcaaaacacctgaaggagaaatcggggccgtgactttggacttccccactggaacatcaggctcaccaatagtggacaaaaacggtgatgtgattgggctttatggcaatggagtcataatgcccaacggctcatacataagcgcgatagtgcagggtgaaaggatggatgagccaatcccagccggattcgaacctgagatgctgaggaaaaaacagatcactgtactggatctccatcccggcgccggtaaaacaaggaggattctgccacagatcatcaaagaggccataaacagaagactgagaacagccgtgctagcaccaaccagggttgtggctgctgagatggctgaagcactgagaggactgcccatccggtaccagacatccgcagtgcccagagaacataatggaaatgagattgttgatgtcatgtgtcatgctaccctcacccacaggctgatgtctcctcacagggtgccgaactacaacctgttcgtgatggatgaggctcatttcaccgacccagctagcattgcagcaagaggttacatttccacaaaggtcgagctaggggaggcggcggcaatattcatgacagccaccccaccaggcacttcagatccattcccagagtccaattcaccaatttccgacttacagactgagatcccggatcgagcttggaactctggatacgaatggatcacagaatacaccgggaagacggtttggtttgtgcctagtgtcaagatggggaatgagattgccctttgcctacaacgtgctggaaagaaagtagtccaattgaacagaaagtcgtacgagacggagtacccaaaatgtaagaacgatgattgggactttgttatcacaacagacatatctgaaatgggggctaacttcaaggcgagcagggtgattgacagccggaagagtgtgaaaccaaccatcataacagaaggagaagggagagtgatcctgggagaaccatctgcagtgacagcagctagtgccgcccagagacgtggacgtatcggtagaaatccgtcgcaagttggtgatgagtactgttatggggggcacacgaatgaagacgactcgaacttcgcccattggactgaggcacgaatcatgctggacaacatcaacatgccaaacggactgatcgctcaattctaccaaccagagcgtgagaaggtatataccatggatggggaataccggctcagaggagaagagagaaaaaactttctggaactgttgaggactgcagatctgccagtttggctggcttacaaggttgcagcggctggagtgtcataccacgaccggaggtggtgctttgatggtcctaggacaaacacaattttagaagacaacaacgaagtggaagtcatcacgaagcttggtgaaaggaagattctgaggccgcgctggattgacgccagggtgtactcggatcaccaggcactaaaggcgttcaaggacttcgcctcgggaaaacgttctcagatagggctcattgaggttctgggaaagatgcctgagcacttcatggggaagacatgggaagcacttgacaccatgtacgttgtggccactgcagagaaaggaggaagagctcacagaatggccctggaggaactgccagatgctcttcagacaattgccttgattgccttattgagtgtgatgaccatgggagtattcttcctcctcatgcagcggaagggcattggaaagataggtttgggaggcgctgtcttgggagtcgcgacctttttctgttggatggctgaagttccaggaacgaagatcgccggaatgttgctgctctcccttctcttgatgattgtgctaattcctgagccagagaagcaacgttcgcagacagacaaccagctagccgtgttcctgatttgtgtcatgacccttgtgagcgcagtggcagccaacgagatgggttggctagataagaccaagagtgacataagcagtttgtttgggcaaagaattgaggtcaaggagaatttcagcatgggagagtttcttttggacttgaggccggcaacagcctggtcactgtacgctgtgacaacagcggtcctcactccactgctaaagcatttgatcacgtcagattacatcaacacctcattgacctcaataaacgttcaggcaagtgcactattcacactcgcgcgaggcttccccttcgtcgatgttggagtgtcggctctcctgctagcagccggatgctggggacaagtcaccctcaccgttacggtaacagcggcaacactccttttttgccactatgcctacatggttcccggttggcaagctgaggcaatgcgctcagcccagcggcggacagcggccggaatcatgaagaacgctgtagtggatggcatcgtggccacggacgtcccagaattagagcgcaccacacccatcatgcagaagaaagttggacagatcatgctgatcttggtgtctctagctgcagtagtagtgaacccgtctgtgaagacagtacgagaagccggaattttgatcacggccgcagcggtgacgctttgggagaatggagcaagctctgtttggaacgcaacaactgccatcggactctgccacatcatgcgtgggggttggttgtcatgtctatccataacatggacactcataaagaacatggaaaaaccaggactaaaaagaggtggggcaaaaggacgcaccttgggagaggtttggaaagaaagactcaaccagatgacaaaagaagagttcactaggtaccgcaaagaggccatcatcgaagtcgatcgctcagcggcaaaacacgccaggaaagaaggcaatgtcactggagggcatccagtctctaggggcacagcaaaactgagatggctggtcgaacggaggtttctcgaaccggtcggaaaagtgattgaccttggatgtggaagaggcggttggtgttactatatggcaacccaaaaaagagtccaagaagtcagagggtacacaaagggcggtcccggacatgaagagccccaactagtgcaaagttatggatggaacattgtcaccatgaagagtggagtggatgtgttctacagaccttctgagtgttgtgacaccctcctttgtgacatcggagagtcctcgtcaagtgctgaggttgaagagcataggacgattcgggtccttgaaatggttgaggactggctgcaccgagggccaagggaattttgcgtgaaggtgctctgcccctacatgccgaaagtcatagagaagatggagctgctccaacgccggtatggggggggactggtcagaaacccactctcacggaattccacgcacgagatgtattgggtgagtcgagcttcaggcaatgtggtacattcagtgaatatgaccagccaggtgctcctaggaagaatggaaaaaaggacctggaagggaccccaatacgaggaagatgtaaacttgggaagtggaaccagggcggtgggaaaacccctgctcaactcagacaccagtaaaatcaagaacaggattgaacgactcaggcgtgagtacagttcgacgtggcaccacgatgagaaccacccatatagaacctggaactatcacggcagttatgatgtgaagcccacaggctccgccagttcgctggtcaatggagtggtcaggctcctctcaaaaccatgggacaccatcacgaatgttaccaccatggccatgactgacactactcccttcgggcagcagcgagtgttcaaagagaaggtggacacgaaagctcctgaaccgccagaaggagtgaagtacgtgctcaatgagaccaccaactggttgtgggcgtttttggccagagaaaaacgtcccagaatgtgctctcgagaggaattcataagaaaggtcaacagcaatgcagctttgggtgccatgtttgaagagcagaatcaatggaggagcgccagagaagcagttgaagatccaaaattttgggagatggtggatgaggagcgcgaggcacatctgcggggggaatgtcacacttgcatttacaacatgatgggaaagagagagaaaaaacccggagagttcggaaaggccaagggaagcagagccatttggttcatgtggctcggagctcgctttctggagttcgaggctctgggttttctcaatgaagaccactggcttggaagaaagaactcaggaggaggtgtcgagggcttgggcctccaaaaactgggttacatcctgcgtgaagttggcacccggcctgggggcaagatctatgctgatgacacagctggctgggacacccgcatcacgagagctgacttggaaaatgaagctaaggtgcttgagctgcttgatggggaacatcggcgtcttgccagggccatcattgagctcacctatcgtcacaaagttgtgaaagtgatgcgcccggctgctgatggaagaaccgtcatggatgttatctccagagaagatcagagggggagtggacaagttgtcacctacgccctaaacactttcaccaacctggccgtccagctggtgaggatgatggaaggggaaggagtgattggcccagatgatgtggagaaactcacaaaagggaaaggacccaaagtcaggacctggctgtttgagaatggggaagaaagactcagccgcatggctgtcagtggagatgactgtgtggtaaagcccctggacgatcgctttgccacctcgctccacttcctcaatgctatgtcaaaggttcgcaaagacatccaagagtggaaaccgtcaactggatggtatgattggcagcaggttccattttgctcaaaccatttcactgaattgatcatgaaagatggaagaacactggtggttccatgccgaggacaggatgaattggtaggcagagctcgcatatctccaggggccggatggaacgtccgcgacactgcttgtctggctaagtcttatgcccagatgtggctgcttctgtacttccacagaagagacctgcggctcatggccaacgccatttgctccgctgtccctgtgaattgggtccctaccggaagaaccacgtggtccatccatgcaggaggagagtggatgacaacagaggacatgttggaggtctggaaccgtgtttggatagaggagaatgaatggatggaagacaaaaccccagtggagaaatggagtgacgtcccatattcaggaaaacgagaggacatctggtgtggcagcctgattggcacaagagcccgagccacgtgggcagaaaacatccaggtggctatcaaccaagtcagagcaatcatcggagatgagaagtatgtggattacatgagttcactaaagagatatgaagacacaactttggttgaggacacagtactgtagatatttaatcaattgtaaatagacaatataagtatgcataaaagtgtagttttatagtagtatttagtggtgttagtgtaaatagttaagaaaattttgaggagaaagtcaggccgggaagttcccgccaccggaagttgagtagacggtgctgcctgcgactcaaccccaggaggactgggtgaacaaagccgcgaagtgatccatgtaagccctcagaaccgtctcggaaggaggaccccacatgttgtaacttcaaagcccaatgtcagaccacgctacggcgtgctactctgcggagagtgcagtctgcgatagtgccccaggaggactgggttaacaaaggcaaaccaacgccccacgcggccctagccccggtaatggtgttaaccagggcgaaaggactagaggttagaggagaccccgcggtttaaagtgcacggcccagcctgactgaagctgtaggtcaggggaaggactagaggttagtggagaccccgtgccacaaaacaccacaacaaaacagcatattgacacctgggatagactaggagatcttctgctctgcacaaccagccacacggcacagtgcgccgacaatggtggctggtggtgcgagaacacaggatct

SEQ ID NO: 67 - protein, 3433 aa, West Nile virus
MSKKPGGPGKSRAVNMLKRGMPRVLSLIGLKRAMLSLIDGKGPIRFVLALLAFFRFTAIAPTRAVLDRWRGVNKQTAMKHLLSFKKELGTLTSAINRRSSKQKKRGGKTGIAVMIGLIASVGAVTLSNFQGKVMMTVNATDVTDVITIPTAAGKNLCIVRAMDVGYMCDDTITYECPVLSAGNDPEDIDCWCTKSAVYVRYGRCTKTRHSRRSRRSLTVQTHGESTLANKKGAWMDSTKATRYLVKTESWILRNPGYALVAAVIGWMLGSNTMQRVVFVVLLLLVAPAYSFNCLGMSNRDFLEGVSGATWVDLVLEGDSCVTIMSKDKPTIDVKMMNMEAANLAEVRSYCYLATVSDLSTKAACPTMGEAHNDKRADPAFVCRQGVVDRGWGNGCGLFGKGSIDTCAKFACSTKAIGRTILKENIKYEVAIFVHGPTTVESHGNYSTQVGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSSIGKAFTTTLKGAQRLAALGDTAWDFGSVGGVFTSVGKAVHQVFGGAFRSLFGGMSWITQGLLGALLLWMGINARDRSIALTFLAVGGVLLFLSVNVHADTGCAIDISRQELRCGSGVFIHNDVEAWMDRYKYYPETPQGLAKIIQKAHKEGVCGLRSVSRLEHQMWEAVKDELNTLLKENGVDLSVVVEKQEGMYKSAPKRLTATTEKLEIGWKAWGKSILFAPELANNTFVVDGPETKECPTQNRAWNSLEVEDFGFGLTSTRMFLKVRESNTTECDSKIIGTAVKNNLAIHSDLSYWIESRLNDTWKLERAVLGEVKSCTWPETHTLWGDGILESDLIIPVTLAGPRSNHNRRPGYKTQNQGPWDEGRVEIDFDYCPGTTVTLSESCGHRGPATRTTTESGKLITDWCCRSCTLPPLRYQTDSGCWYGMEIRPQRHDEKTLVQSQVNAYNADMIDPFQLGLLVVFLATQEVLRKRWTAKISMPAILIALLVLVFGGITYTDVLRYVILVGAAFAESNSGGDVVHLALMATFKIQPVFMVASFLKARWTNQENILLMLAAVFFQMAYHDARQILLWEIPDVLNSLAVAWMILRAITFTTTSNVVVPLLALLTPGLRCLNLDVYRILLLMVGIGSLIREKRSAAAKKKGASLLCLALASTGLFNPMILAAGLIACDPNRKRGWPATEVMTAVGLMFAIVGGLAELDIDSMAIPMTIAGLMFAAFVISGKSTDMWIERTADISWESDAEITGSSERVDVRLDDDGNFQLMNDPGAPWKIWMLRMVCLAISAYTPWAILPSVVGFWITLQYTKRGGVLWDTPSPKEYKKGDTTTGVYRIMTRGLLGSYQAGAGVMVEGVFHTLWHTTKGAALMSGEGRLDPYWGSVKEDRLCYGGPWKLQHKWNGQDEVQMIVVEPGKNVKNVQTKPGVFKTPEGEIGAVTLDFPTGTSGSPIVDKNGDVIGLYGNGVIMPNGSYISAIVQGERMDEPIPAGFEPEMLRKKQITVLDLHPGAGKTRRILPQIIKEAINRRLRTAVLAPTRVVAAEMAEALRGLPIRYQTSAVPREHNGNEIVDVMCHATLTHRLMSPHRVPNYNLFVMDEAHFTDPASIAARGYISTKVELGEAAAIFMTATPPGTSDPFPESNSPISDLQTEIPDRAWNSGYEWITEYTGKTVWFVPSVKMGNEIALCLQRAGKKVVQLNRKSYETEYPKCKNDDWDFVITTDISEMGANFKASRVIDSRKSVKPTIITEGEGRVILGEPSAVTAASAAQRRGRIGRNPSQVGDEYCYGGHTNEDDSNFAHWTEARIMLDNINMPNGLIAQFYQPEREKVYTMDGEYRLRGEERKNFLELLRTADLPVWLAYKVAAAGVSYHDRRWCFDGPRTNTILEDNNEVEVITKLGERKILRPRWIDARVYSDHQALKAFKDFASGKRSQIGLIEVLGKMPEHFMGKTWEALDTMYVVATAEKGGRAHRMALEELPDALQTIALIALLSVMTMGVFFLLMQRKGIGKIGLGGAVLGVATFFCWMAEVPGTKIAGMLLLSLLLMIVLIPEPEKQRSQTDNQLAVFLICVMTLVSAVAANEMGWLDKTKSDISSLFGQRIEVKENFSMGEFLLDLRPATAWSLYAVTTAVLTPLLKHLITSDYINTSLTSINVQASALFTLARGFPFVDVGVSALLLAAGCWGQVTLTVTVTAATLLFCHYAYMVPGWQAEAMRSAQRRTAAGIMKNAVVDGIVATDVPELERTTPIMQKKVGQIMLILVSLAAVVVNPSVKTVREAGILITAAAVTLWENGASSVWNATTAIGLCHIMRGGWLSCLSITWTLIKNMEKPGLKRGGAKGRTLGEVWKERLNQMTKEEFTRYRKEAIIEVDRSAAKHARKEGNVTGGHPVSRGTAKLRWLVERRFLEPVGKVIDLGCGRGGWCYYMATQKRVQEVRGYTKGGPGHEEPQLVQSYGWNIVTMKSGVDVFYRPSECCDTLLCDIGESSSSAEVEEHRTIRVLEMVEDWLHRGPREFCVKVLCPYMPKVIEKMELLQRRYGGGLVRNPLSRNSTHEMYWVSRASGNVVHSVNMTSQVLLGRMEKRTWKGPQYEEDVNLGSGTRAVGKPLLNSDTSKIKNRIERLRREYSSTWHHDENHPYRTWNYHGSYDVKPTGSASSLVNGVVRLLSKPWDTITNVTTMAMTDTTPFGQQRVFKEKVDTKAPEPPEGVKYVLNETTNWLWAFLAREKRPRMCSREEFIRKVNSNAALGAMFEEQNQWRSAREAVEDPKFWEMVDEEREAHLRGECHTCIYNMMGKREKKPGEFGKAKGSRAIWFMWLGARFLEFEALGFLNEDHWLGRKNSGGGVEGLGLQKLGYILREVGTRPGGKIYADDTAGWDTRITRADLENEAKVLELLDGEHRRLARAIIELTYRHKVVKVMRPAADGRTVMDVISREDQRGSGQVVTYALNTFTNLAVQLVRMMEGEGVIGPDDVEKLTKGKGPKVRTWLFENGEERLSRMAVSGDDCVVKPLDDRFATSLHFLNAMSKVRKDIQEWKPSTGWYDWQQVPFCSNHFTELIMKDGRTLVVPCRGQDELVGRARISPGAGWNVRDTACLAKSYAQMWLLLYFHRRDLRLMANAICSAVPVNWVPTGRTTWSIHAGGEWMTTEDMLEVWNRVWIEENEWMEDKTPVEKWSDVPYSGKREDIWCGSLIGTRARATWAENIQVAINQVRAIIGDEKYVDYMSSLKRYEDTTLVEDTVL

SEQ ID NO: 68 - DNA, 32 bp, Artificial Sequence
  Description of Artificial Sequence Synthetic primer
ggccgaattctgaatgttaaatgttatacttt

SEQ ID NO: 69 - DNA, 66 bp, Artificial Sequence
  Description of Artificial Sequence Synthetic primer
cccgggatcgatggatcctttttatagctaattagtcacgtacctttgagagtaccacttcagcta

SEQ ID NO: 70 - DNA, 66 bp, Artificial Sequence
  Description of Artificial Sequence Synthetic primer
ggatccatcgatcccgggtttttatgactagttaatcacggccgcttataaagatctaaaatgcat

SEQ ID NO: 71 - DNA, 30 bp, Artificial Sequence
  Description of Artificial Sequence Synthetic primer
ggctgcaggtattctaaactaggaatagat

SEQ ID NO: 72 - DNA, 12 bp, Artificial Sequence
  Description of Artificial Sequence Synthetic oligonucleotide
aattgcggccgc

SEQ ID NO: 73 - DNA, 29 bp, Artificial Sequence
  Description of Artificial Sequence Synthetic primer
aaaggatccgggttaattaattagtcatc

SEQ ID NO: 74 - DNA, 34 bp, Artificial Sequence
  Description of Artificial Sequence Synthetic primer
aataaagaagctctaattaattaacgagcagata

SEQ ID NO: 75 - DNA, 41 bp, Artificial Sequence
  Description of Artificial Sequence Synthetic primer
tcgttaattaattagagcttctttattctatacttaaaaag

SEQ ID NO: 76 - DNA, 56 bp, Artificial Sequence
  Description of Artificial Sequence Synthetic primer
aaaacccgggatcgattctagactcgagggtacctacgatacaaacttaacggata

SEQ ID NO: 77 - DNA, 5000 bp, Canarypox virus
  CDS (1864)..(2184)
ctgaaattgtaatttctacatgtagagaaggttttgatattgatggttttaacagaaacgtagaaattatatcaagggataacattttatatgatatagttttaaagtgtaagatggaattaaatttcatgtgcacaagaggcataggagataaaagcattttcagactttgtataatgaaggaatatgatcaaataaacaagaatctgttagttagttacttggataaattaatcgagacgcgtgataaaatgactatgtaccgttattgcatgaacgatattataaatataggttctcgtaggagagaactattgactatggcaatgaatgttaaatgttatactttggatgaagctataaatatgcattggaaaaataatccatttaaagaaaggattcaaatactacaaaacctaagcgataatatgttaactaagcttattcttaacgacgctttaaatatacacaaataaacataatttttgtataacctaacaaataactaaaacataaaaataataaaaggaaatgtaatatcgtaattattttactcaggaatggggttaaatatttatatcacgtgtatatctatactgttatcgtatactctttacaattactattacgaatatgcaagagataataagattacgtatttaagagaatcttgtcatgataattgggtacgacatagtgataaatgctatttcgcatcgttacataaagtcagttggaaagatggatttgacagatgtaacttaataggtgcaaaaatgttaaataacagcattctatcggaagataggataccagttatattatacaaaaatcactggttggataaaacagattctgcaatattcgtaaaagatgaagattactgcgaatttgtaaactatgacaataaaaagccatttatctcaacgacatcgtgtaattcttccatgttttatgtatgtgtttcagatattatgagattactataaactttttgtatacttatattccgtaaactatattaatcatgaagaaaatgaaaaagtatagaagctgttcacgagcggttgttgaaaacaacaaaattatacattcaagatggcttacatatacgtctgtgaggctatcatggataatgacaatgcatctctaaataggtttttggacaatggattcgaccctaacacggaatatggtactctacaatctcctcttgaaatggctgtaatgttcaagaataccgaggctataaaaatcttgatgaggtatggagctaaacctgtagttactgaatgcacaacttcttgtctgcatgatgcggtgttgagagacgactacaaaatagtgaaagatctgttgaagaataactatgtaaacaatgttctttacagcggaggctttactcctttgtgtttggcagcttaccttaacaaagttaatttggttaaacttctattggctcattcggcggatgtagatatttcaaacacggatcggttaactcctctacatatagccgtatcaaataaaaatttaacaatggttaaacttctattgaacaaaggtgctgatactgacttgctggataacatgggacgtactcctttaatgatcgctgtacaatctggaaatattgaaatatgtagcacactacttaaaaaaaataaaatgtccagaactgggaaaaattgatcttgccagctgtaattcatggtagaaaagaagtgctcaggctacttttcaacaaaggagcagatgtaaactacatctttgaaagaaatggaaaatcatatactgttttggaattgattaaagaaagttactctgagacacaaaagaggtagctgaagtggtactctcaaaatgcagaacgatgactgcgaagcaagaagtagagaaataacactttatgactttcttagttgtagaaaagatagagatataatgatggtcataaataactctgatattgcaagtaaatgcaataataagttagatttatttaaaaggatagttaaaaatagaaaaaaagagttaatttgtagggttaaaataatacataagatcttaaaatttataaatacgcataataataaaaatagattatacttattaccttcagagataaaatttaagatatttacttatttaacttataaagatctaaaatgcataatttctaaataatgaaaaaaagtacatcatgagcaacgcgttagtatattttacaatggagattaacgctctataccgttctatgtttattgattcagatgatgttttagaaaagaaagttattgaatatgaaaactttaatgaagatgaagatgacgacgatgattattgttgtaaatctgttttagatgaagaagatgacgcgctaaagtatactatggttacaaagtataagtctatactactaatggcgacttgtgcaagaaggtatagtatagtgaaaatgttgttagattatgattatgaaaaaccaaataaatcagatccatatctaaaggtatctcctttgcacataatttcatctattcctagtttagaatacttttcattatatttgtttacagctgaagacgaaaaaaatatatcgataatagaagattatgttaactctgctaataagatgaaattgaatgagtctgtgataatagctataatcagagaagttctaaaaggaaataaaaatctaactgatcaggatataaaaacattggctgatgaaatcaacaaggaggaactgaatatagctaaactattgttagatagaggggccaaagtaaattacaaggatgtttacggttcttcagctctccatagagctgctattggtaggaaacaggatatgataaagctgttaatcgatcatggagctgatgtaaactctttaactattgctaaagataatcttattaaaaaaaaataatatcacgtttagtaatattaaaatatattaataactctattactaataactccagtggatatgaacataatacgaagtttatacattctcatcaaaatcttattgacatcaagttagattgtgaaaatgagattatgaaattaaggaatacaaaaataggatgtaagaacttactagaatgttttatcaataatgatatgaatacagtatctagggctataaacaatgaaacgattaaaaattataaaaatcatttccctatatataatacgctcatagaaaaattcatttctgaaagtatactaagacacgaattattggatggagttataaattcttttcaaggattcaataataaattgccttacgagattcagtacattatactggagaatcttaataaccatgaactaaaaaaaattttagataatatacattaaaaaggtaaatagatcatctgttattataagcaaagatgcttgttgccaataatatacaacaggtatttgtttttatttttaactacatatttgatgttcattctctttatatagtatacacagaaaattcataatccacttagaatttctagttatctagtttttctagaatattgtactttatttctaatggaatggctctccagcctagtaatttattaatgttagctgatatcttgaaatcaggatattctgctccgtgaagagaaagtcctccaaagttgtatatttccatcactttcatggcttcctctttccatagtgtcttctataagctgtctatatattgtaaacttttctggttttatgcattttaaacatttagcaatctcattttcatcacaattaaggcacaaatctaacatggaatgtctaccataacccaataaggttttttcatttcctctatctctaatacacactgttctttccagactttcaacacgctgctattttctattttattcaagtccatattataagcgtccttgttagacacttcataatgtttgcattctggaatcatcatgttagatactattaatttagctacttctatgttgtcatcaaaagagttgctatctgtaattacactaagaggtgtatcacctgataaagaagtaatagagacatctgctctgaatttaagcaatacctcaataacttcttttgaagatgactttgcagctaaaaataatggagttctctctaaaacatcccttgagtttatattagccccgtaactaatgagtagttctgttatatctttggaatctattgatatattattaattacaattgtcatgctgacatatatagacatcataatatgatgaaaaatatgaaaatataagtgcacgtttactgttactatgattgtgatatcgatatgagttctttaataaaagtactgaaatagatataatgcagatatgattgatattttaaaaagttgaaaaaaaatatgccctgtttacaaatactatttggaaatattctgtaataaagtaatagtgatatgtcagtcacgatggatttgccaattgatcatatgagtatagataacataaacgagtataataaaaatggatatactagactctatatagaggtagccatgaaaaaacgtaaaaacgtagatagacttttatatctcggagctgatccgaatctggctagtgtagattcgtattgtcctcttcatattgctgttaggaatggtagtttaaagataataagatcattgttgaaatatggtgctaatataaatcaagaatgtcatgaaggagatactgctttgatgatggctatatcattaggtaattatacagcatgtaaaacacttctagataacaacgccgatcctaattatgttaactattacggtatagttccgcttattagagcaattatatgtgaaaagcctgacatagttagactgctattagatagaggagctaattgcaaccacttaattacaaaaaacggtagaacctatactgctttagagagtcttaggaattgcttttttaaagacaattcttcatcattgtcgatactaatat

SEQ ID NO: 78 - protein, 107 aa, Canarypox virus
MQNDDCEARSREITLYDFLSCRKDRDIMMVINNSDIASKCNNKLDLFKRIVKNRKKELICRVKIIHKILKFINTHNNKNRLYLLPSEIKFKIFTYLTYKDLKCIISK

SEQ ID NO: 79 - DNA, 10 bp, Artificial Sequence
  Description of Artificial Sequence Synthetic primer
ggccgaattc

SEQ ID NO: 80 - DNA, 36 bp, Artificial Sequence
  Description of Artificial Sequence Synthetic primer
ccatgcactgattaatcgatatttttccatgggccc

SEQ ID NO: 81 - DNA, 36 bp, Artificial Sequence
  Description of Artificial Sequence Synthetic primer
ggatcccgggtttttatgactagttaatcacggccg

SEQ ID NO: 82 - protein, 12 aa, Artificial Sequence
  Description of Artificial Sequence Synthetic VQ marker
VIRQGENETICS

SEQ ID NO: 83 - DNA, 232 bp, Artificial Sequence
  Description of Artificial Sequence Synthetic VQH6 amplified nucleotide fragment
aaaggatccgggttaattaattagtcatcaggcagggcgagaacgagactatctgctcgttaattaattagagcttctttattctatacttaaaaagtgaaaataaatacaaaggttcttgagggttgtgttaaattgaaagcgagaaataatcataaattatttcattatcgcgatatccgttaagtttgtatcgtaggtaccctcgagtctagaatcgatcccgggtttt

SEQ ID NO: 84 - DNA, 14 bp, Artificial Sequence
  Description of Artificial Sequence Synthetic oligonucleotide
gccgccaccatggg

SEQ ID NO: 85 - DNA, 18 bp, Artificial Sequence
  Description of Artificial Sequence Synthetic oligonucleotide
cccatggtggcggctgca

(19) United States
(12) Patent Application Publication
(10) Pub. No.: US 2005/0031641 A1
(43) Pub. Date: Feb. 10, 2005

(54) Recombinant vaccine against West Nile Virus

(76) Inventors: Sheena May Loosmore, Aurora (CA); Jean-Christophe Francis Audonnet, Lyon (FR); Jules Maarten Minke, Corbas (FR); Kemal Karaca, Athens, GA (US)

Correspondence Address: FROMMER LAWRENCE & HAUG, 745 FIFTH AVENUE- 10TH FL., NEW YORK, NY, 10151, US

(21) Appl. No.: 10/679,520

(22) Filed: Oct. 6, 2003

Related U.S. Application Data
(63) Continuation-in-part of application No. 10/374,953, filed on Feb. 26, 2003.
Application No. 10/374,953 is a continuation-in-part of application No. 10/116,298, filed on Apr. 4, 2002, now abandoned.
(63) Continuation-in-part of application No. PCT/FR02/01200, filed on Apr. 5, 2002.
(60) Provisional application No. 60/281,923, filed on Apr. 6, 2001.

(30) Foreign Application Priority Data
Apr. 6, 2001 (FR) 01/04737

(51) Int. Cl. A61K 48/00; A61K 39/12

(52) U.S. Cl. 424/199.1; 514/44

(57) ABSTRACT

An immunogenic or vaccine composition to induce an immune response or protective immune response against West Nile virus (WNV) in an animal susceptible to WNV. The composition includes a pharmaceutically or veterinarily acceptable vehicle or excipient, and a vector. The vector contains heterologous nucleic acid molecule(s), expresses in vivo in the animal WNV antigen, immunogen or epitope thereof, e.g., WNV E; WNV prM and E; WNV M and E; WNV prM, WNV M and E, WNV polyprotein prM-E, WNV polyprotein M-E, or WNV polyprotein prM-M-E. The composition can contain an adjuvant, such as carbomer. Methods for making and using such a composition, including prime-boost regimes and including as to differential diagnosis, are also contemplated.

### RELATED APPLICATIONS/INCORPORATION BY REFERENCE

This application is a continuation-in-part application of U.S. application Ser. No. 10/374,953 filed Feb. 26, 2003, which is a continuation-in-part of U.S. application Ser. No. 10/116,298, filed Apr. 4, 2002, which claims priority from U.S. Provisional application Ser. No. 60/281,923, filed Apr. 6, 2001. This application is also a continuation-in-part application of U.S. application Ser. No. ______ filed Sep. 30, 2003 (Attorney Docket No. 454313-3161.3), which is a continuation of U.S. application Ser. No. 10/374,953 filed Feb. 26, 2003, and which claims priority from U.S. Provisional Application Ser. No. 60/281,923, filed Apr. 6, 2001. This application is also a continuation-in-part of International Application PCT/FR02/01200 filed Apr. 5, 2002, published as WO 02/081621 on Oct. 17, 2002, which claims priority to French application 01/04737 filed Apr. 6, 2001. Each of the above applications, together with each document cited therein, and each of the documents referenced or cited in documents cited therein, are hereby incorporated herein by reference.

Indeed, more generally, each document cited in this text (“application cited documents”) and each document cited or referenced in each of the application cited documents, and any manufacturer's specifications or instructions for any products mentioned in this text and in any document incorporated into this text, are hereby incorporated herein by reference; and, technology in each of the documents incorporated herein by reference can be used in the practice of this invention.

### FIELD OF THE INVENTION

The present invention relates to vectors containing at least one polynucleotide of the West Nile fever virus (or West Nile Virus or WNV) or at least one nucleic acid molecule encoding at least one West Nile Virus antigen, immunogen or epitope, e.g., in vivo and in vitro expression vectors comprising and expressing at least one polynucleotide of the West Nile Virus or in vivo and in vitro expression vectors comprising and expressing at least one West Nile Virus antigen, immunogen or epitope, as well as immunogenic compositions and vaccines against West Nile fever; for instance, such compositions or vaccines that contain one or more of the vectors and/or one or more of the expression products of the vectors. The invention also relates to methods for using the vectors, compositions and vaccines, including for immunizing and vaccinating against this virus, expressing expression products of the polynucleotide(s), using the expression products in assays or to generate antibodies useful in assays, as well as to methods for making the, polynucleotide(s), vectors, compositions vaccines, assays, inter alia.

### BACKGROUND OF THE INVENTION

The West Nile fever virus (WNV) was first identified in man in 1937 in Uganda in the West Nile Province (Zeller H. G., Med. Trop., 1999, 59, 490-494).

Widespread in Africa, it is also found in India, Pakistan and the Mediterranean basin and was identified for the first time in the USA in 1999 in New York City (Anderson J. F. et al., Science, 1999, 286, 2331-2333).

The West Nile fever virus affects birds as well as reptiles, mammals, together with man.

The disease is characterized in birds by an attack of the central nervous system and death. The lesions include encephalitis, hemorrhages in the myocardium and hemorrhages and necroses in the intestinal tract.

In chickens, experimental infections by subcutaneous inoculations of the West Nile fever virus isolated on crows led to necrosis of the myocardium, nephritis and pneumonia 5 to 10 days after inoculation and moderate to severe encephalitis 21 days after inoculation (Senne D. A. et al., Avian Disease, 2000, 44, 642-649).

The West Nile fever virus also affects horses, especially in North Africa and Europe (Cantile C. et al., Equine Vet. J., 2000, 32 (1), 31-35). These horses reveal signs of ataxia, weakness of the rear limbs, paresis evolving towards tetraplegia and death. Horses and camels are the main animals manifesting clinical signs in the form of encephalitis.

Anti-WNV antibodies were detected in certain rodents, in livestock, especially bovines and ovines, as well as in domestic animals, especially in the dog (Zeller H. G., Med. Trop., 1999, 59, 490-494; Lundstrom J. O., Journal of Vector Ecology, 1999, 24 (1), 1-39).

The West Nile fever virus also affects with a number of symptoms the human species (Sampson B. A., Human Pathology, 2000, 31 (5), 527-531; Marra C. M., Seminars in Neurology, 2000, 20 (3), 323-327).

The West Nile fever virus is transmitted to birds and mammals by the bites of certain mosquitoes (e.g. Culex, Aedes, Anopheles). Direct transmission may happen from WNV infected subject to healthy subject by oral transmission (prey and transmission through colostrum) and blood/organ vectored transmission.

Wild and domestic birds are a reservoir for the West Nile virus and a propagation vector as a result of their migrations.

The virions of the West Nile fever virus are spherical particles with a diameter of 50 nm constituted by a lipoproteic envelope surrounding an icosahedric nucleocapsid containing a positive polarity, single-strand RNA.

A single open reading frame (ORF) encodes all the viral proteins in the form of a polyprotein. The cleaving and maturation of this polyprotein leads to the production of about ten different viral proteins. The structural proteins are encoded by the 5′ part of the genome and correspond to the nucleocapsid designated C (14 kDa), the envelope glycoprotein designated E (50 kDa), the pre-membrane protein designated prM (23 kDa), the membrane protein designated M (7 kDa). The non-structural proteins are encoded by the 3′ part of the genome and correspond to the proteins NS1 (40 kDa), NS2A (19 kDa), NS2B (14 kDa), NS3 (74 kDa), NS4A (15 kDa), NS4B (29 kDa), NS5 (97 kDa).

Parrish C. R. et al. (J. Gen. Virol., 1991, 72, 1645-1653), Kulkami A. B. et al. (J. Virol., 1992, 66 (6), 3583-3592) and Hill A. B. et al. (J. Gen. Virol., 1992, 73, 1115-1123), on the basis of the vaccinia virus, constructed in vivo expression vectors containing various inserts corresponding to nucleotide sequences coding for non-structural proteins of the Kunjin virus, optionally associated with structural proteins. These vectors were administered to mice to evaluate the immune cell response. The authors stress the importance of the cell response, which is essentially stimulated by non-structural proteins and especially NS3, NS4A and NS4B. These articles reveal the difficulty in providing a good vaccination strategy against West Nile fever.

Reference is also made to WO 02/081754 published Oct. 17, 2002, from PCT/US02/10764, filed Apr. 4, 2002, with a claim of priority from U.S. application Ser. No. 09/826,115, filed Apr. 4, 2001. The PCT claims a status of continuation-in-part from U.S. application Ser. No. 09/826,115. It further states that U.S. application Ser. No. 09/826,115 is a continuation-in-part of U.S. application Ser. No. 09/701,536, filed Nov. 29, 2000. It even further states that U.S. application Ser. No. 09/701,536 is the National Stage of PCT/US99/12298, filed Jun. 3, 1999, with a claim of priority to U.S. provisional application Ser. No. 60/087,908.

It would be advantageous to provide improved immunogenic and vaccine compositions against WNV, and methods for making and using such compositions, including such compositions that provide for differential diagnostic methods, assays and kits, and thus, differential diagnostic methods, assays and kits.

### OBJECTS AND/OR SUMMARY OF THE INVENTION

The invention provides an immunogenic or vaccine composition to induce an immune response or protective immune response against West Nile virus (WNV) in an animal susceptible to WNV comprising or consisting essentially of a pharmaceutically or veterinarily acceptable vehicle or excipient and a vector that contains or consists essentially of heterologous nucleic acid molecule(s), and that expresses in vivo in the animal a WNV protein, antigen, immunogen or epitope thereof, such as WNV E; WNV prM and E; WNV M and E; WNV prM, WNV M and E, WNV polyprotein prM-E, WNV polyprotein M-E, or WNV polyprotein prM-M-E.

The vector can be a DNA plasmid or a recombinant virus, such as a recombinant adenovirus, herpesvirus or poxvirus, e.g., an avipox virus, such as a canarypox virus or a fowlpox virus. The animal can be selected from the group consisting of an equine, a canine, a feline, a bovine, a porcine, a chicken, a duck, a goose and a turkey.

Advantageously, the nucleic acid molecule comprises or consists essentially of nucleotides 466-741, 742-966 and 967-2469 of GenBank AF196835 encoding WNV prM, M and E, respectively, nucleotides 466-2469 of GenBank AF196835 encoding WN protein prM-M-E, or nucleotides 421-2469 of GenBank AF196835 encoding WN protein prM-M-E and the signal peptide of prM.

The immunogenic or vaccine composition can further comprise or consist essentially of an adjuvant, such as a carbomer.

The immunogenic or vaccine composition can further comprise or consist essentially of an antigen or immunogen or epitope thereof of a pathogen other than WNV of the animal, or a vector that contains and expresses in vivo in the animal a nucleic acid molecule encoding the antigen, immunogen or epitope thereof, or an inactivated or attenuated pathogen other than WNV of the animal.

The invention additionally involves a kit comprising or consisting essentially of (a) the immunogenic or vaccine composition, and (b) the antigen or immunogen or epitope thereof of a pathogen other than WNV of the animal, or the vector that contains and expresses in vivo in the animal a nucleic acid molecule encoding the antigen, immunogen or epitope thereof, or the inactivated or attenuated pathogen other than WNV of the animal, wherein (a) and (b) are in separate containers, and the kit optionally contains instructions for admixture and/or administration of (a) and (b).

The invention also comprehends a method for inducing an immunological or protective immune response against WNV in an animal comprising or consisting essentially of administering to the animal the immunogenic or vaccine composition.

The invention further comprehends a method for inducing an immunological or protective immune response against WNV in an animal comprising or consisting essentially of administering to the animal (a) the immunogenic or vaccine composition, and (b) a WNV isolated antigen, immunogen or epitope thereof, wherein (a) is administered prior to (b) in a prime-boost regimen, or (b) is administered prior to (a) in a prime-boost regimen, or (a) and (b) are administered together, either sequentially or in admixture. The invention also involves a kit for performing this comprising or consisting essentially of (a) and (b) in separate containers, optionally with instructions for admixture and/or administration.

The invention even further comprehends a prime-boost immunization or vaccination against WNV, wherein the priming is done with (a) DNA vaccine(s) or immunological or immunogenic composition(s) that contains or consists essentially of (a) nucleic acid molecule(s) encoding and express(es) in vivo a WNV immunogen, antigen or epitope and the boost is done with (a) vaccine(s) or immunological or immunogenic composition(s) that is a WNV inactivated or attenuated or subunit (antigen, immunogen and/or epitope) preparation(s) and/or (a) recombinant or modified virus vaccine or immunological or immunogenic composition(s) that contains or consists essentially of (a) nucleic acid mocule encoding and express(es) in vivo (a) WNV immunogen(s), antigen(s) or epitope(s). Thus, the invention provides a prime-boost immunization or vaccination method against WNV, such as a prime-boost immunization or vaccination which comprises or consists essentially of or consists of administering to a target species animal (a) DNA vaccine(s) or immunological or immunogenic composition(s) of the invention (that contains or consists essentially of nucleic acid molecule(s) encoding and express(es) in vivo WNV antigen(s), immunogen(s) or epitope(s)) (as the prime) and thereafter administering (as the boost) administering inactivated WNV and/or attenuated WNV or a WNV subunit (antigen, immunogen and/or epitope) preparation(s)) and/or a recombinant or modified virus vaccine or immunological or immunogenic composition that contains or consists essentially of nucleic acid molecule(s) encoding and expresse(s) in vivo WNV immunogen(s), antigen(s) or epitope(s), advantageously (a) recombinant vaccine or immunological or immunogenic composition(s) that expresses the WNV immunogen, antigen or epitope in vivo. The boost is advantageously matched to the prime, e.g., the boost contains or consists essentially of or expresses at least one antigen, epitope or immunogen that is expressed by the prime.

The prime-boost regimen according to the invention can be used in animals of any age, advantageously young animals (e.g., animals that have detectable maternal antibodies and/or are suckling or nursing or breast-feeding), pre-adult animals (animals that are older than being a young animal but have not yet reached maturity or adulthood or an age to mate or reproduce), adult animals (e.g., animals that are of an age to mate or reproduce or are beyond such a period in life), and it is advantageous to employ the prime-boost regimen in pregnant females or females prior to giving birth, laying, or insemination.

The invention also relates to such immunogenic and vaccine compositions and kits thereof suitable for use in such prime-boost regimens and prime-boost regimens. The host or target species upon which the prime-boost regimen can be practiced includes any animal (target or host) species susceptible to disease caused by WNV, including mammals, reptiles, birds, especially humans, companion mammals or animals such as canines, felines, equines, zoo mammals or animals, such as aquatic mammals e.g. seals, felines, equines, zoo reptiles such as snakes, crocodiles, aligators, and avian species, such as domesticated birds that are pets or poultry, or wild birds, e.g., canaries, parakeets, chickens, ducks, geese, turkeys, sparrows, crows, and the like.

The prime-boost regimen is especially advantageous to practice in a young animal, as it allows vaccinatation or immunization at an early age, for instance, the first administration in the prime-boost regimen when practiced on a young animal can be at an age at which the the young animal has maternal antibodies. Another advantage of this regimen is that it can provide a degree of safety for pregnant females present in the same location or in close proximity to the young or to each other. Thus, the invention provides a prime-boost immunization or vaccination method against WNV, and the method may be practiced upon a young animal, such as a young foal, puppy or kitten, for instance, wherein the priming is done at a time that the young animal has maternal antibodies against WNV, with the boost advantageously at a time when maternal antibodies may be waning or decreasing or normally not present, such as a period of time post-breastfeeding.

Accordingly, the invention also involves kits for performing a prime-boost regimen comprising or consisting essentially of a priming vaccine or immunological or immunogenic composition and a boost vaccine or immunological or immunogenic compositions, in separate containers, optionally with instructions for admixture and/or administration.

Further still, the invention provides a differential diagnosis method comprising administering to animals an immunogenic or vaccine composition and/or a WNV antigen, immunogen or epitope, and testing the animals for presence or absence of a WNV protein or antibody thereto not expressed by the immunogenic or vaccine composition and/or not present in the WNV antigen, immunogen or epitope. An the invention additionally involves a kit for performing this method comprising the immunogenic or vaccine composition and/or the WNV antigen, immunogen or epitope, and an assay for testing for the presence or absence of the WNV protein, in separate containers, optionally with instructions for administration of the immunogenic or vaccine composition and/or the WNV antigen, immunogen or epitope and/or for performing the assay.

These and other embodiments are disclosed or are obvious from and encompassed by, the following Detailed Description.

### BRIEF DESCRIPTION OF THE DRAWINGS

FIG. 1 is a schematic showing construction of a pC5 H6p WNV prM-M-E donor plasmid, pDS-2946-1-1.

FIG. 2 depicts the nucleic and amino acid sequence of C5H6p WNV prM-M-E C5 in pDS-2646-1-1 (SEQ ID Nos: 54 and 55).

FIG. 3 is a schematic showing construction of a pF8 H6p WNV prM-M-E donor plasmid, pSL-5513-1-1-1.

FIG. 4 depicts the nucleic and amino acid sequence of F8 H6p WNV prM-M-E F8 in pSL-5513-1-1-1 (SEQ ID Nos: 56 and 57).

FIG. 5 is two immunoblots showing the expression of WNV proteins from pox recombinants in chick embryo fibroblast cells.

FIG. 6 is an immunoblot showing the expression of WNV proteins from pox recombinants in BHK cells.

FIG. 7 is a schematic showing construction of pVR1012 WNV prM-M-E, pSL-5448-1-1.

FIG. 8 depicts the nucleic and amino acid sequences of the WNV prM-M-E region in pSL-5448-1-1, pVR1012 WNV prM-M-E (SEQ ID Nos: 58 and 59).

FIG. 9 depicts the nucleic and amino acid sequences of pDS-2946-1-1, pC5 H6p WNV prM-M-E (SEQ ID Nos: 60 and 61).

FIG. 10 is a schematic showing construction of pC5 H6p WNV prM-M-E donor plasmids with a truncated H6p and/or truncated WNV capsid leader sequence.

FIG. 11 describes primers for constructions pC5 H6p WNV prM-M-E donor plasmids with a truncated H6p and/or truncated WNV capsid leader sequence (SEQ ID Nos: 48, 49, 50, 62, 63, 64 and 65). Additionally, the full length sequence of H6p 5′WNV sequence in vCP2017 is depicted (portions of SEQ ID Nos: 60 and 61).

FIG. 12 depicts the nucleic and amino acid sequences of the West Nile Virus (WNV) (SEQ ID Nos: 66 and 67).

### DETAILED DESCRIPTION

As discussed herein, the present invention relates to vectors containing at least one polynucleotide of the West Nile fever virus (or West Nile Virus or WNV) or at least one nucleic acid molecule encoding at least one West Nile Virus antigen, immunogen or epitope, e.g., in vivo and in vitro expression vectors comprising and expressing at least one polynucleotide of the West Nile Virus or in vivo and in vitro expression vectors comprising and expressing at least one West Nile Virus antigen, immunogen or epitope, as well as immunogenic compositions and vaccines against West Nile fever; for instance, such compositions or vaccines that contain one or more of the vectors and/or one or more of the expression products of the vectors.

Advantageously, the immunogen or antigen is the envelope protein, E, or the pre-membrane protein (prM protein), or the membrane protein (M protein), or combinations thereof, e.g., E and prM; E and M; E and prM and M; prM and M. The combinations can be separate proteins or polyproteins. The compositions or vaccines can thus contain one or more vectors expressing more than one of the proteins, e.g., different proteins. The compositions or vaccines can contain, or vectors thereof express, proteins from different strains or isolates of WNV. Thus, the compositions or vaccines can contain, or the vectors thereof express, E, prM, M or combinations thereof, wherein the E, prM, and/or M are from different strains or isolates.

In this regard, it is noted that there is the NYC isolate or strain, e.g., WN-NY99 strain or GenBank AF196835 (WNV isolated from a dead Chilean flamingo at the Bronx Zoo deposited in GenBank, R. S. Lanciotti et al., Science, 286, pp. 2333-7 (1999); SEQ ID Nos: 66 and 67) or GenBank AAF202541 (genome of a WNV isolate from human victims of the New York outbreak of WNV-NY1999, X-Y. Jia et al., The Lancet, 354, pp. 1971-2 (1999)) (see also Ebel et al., Emerg Infect Dis 7(4):650-3 (2001), Anderson et al. PNAS USA 98(23):12885-9 (2001), Shi et al., Virology 296(2):219-33 (2002), Shi et al., J Virol 76(12):5847-56 (2002)), as well as the strains of GenBank D00246 (Kunjin virus); M12294 (West Nile virus); AF130362 (West Nile virus strain RO97-50); AF130363 (West Nile virus strain 96-1030)). Also, it is noted that comparative phylogenetic analysis of the NY sequences with previously reported WNV sequences indicated a high degree of homology between the NY isolates and two isolates from Romania and one from Israel (J. F. Anderson et al., supra; X.-Y. Jia et al., supra; R. S. Lanciotti et al., supra), indicating the useful of the NY sequences.

Advantageously in embodiments involving at least one epitope present in, or expressed by vector or vectors in, compositions or vaccines of the invention, the epitope or epitopes are from E, prM, M or combinations thereof, and the epitope or epitopes can be from different strains or isolates. In this regard, it is noted that one can locate or map epitopes in WNV antigens or immunogens, such as the E protein; see, e.g., Beasley et al. J Virol 76(24): 13097-100 (2002), Damle et al. Acta Virol 42(6):389-95 (1998), De Groot et al., Emerg Infect Dis 7(4):706-13 (2001), Sbai et al., Curr Drug Targets Infect Disord 1(3):303-13 (2001), Kutubuddin et al., Mol Immunol 28(1-2):149-54 (1991), Becker, Virus Genes 4(3):267-82 (1990).

Also as discussed herein, the invention relates to methods for using the vectors, compositions and vaccines, including for immunizing and vaccinating against this virus, for expressing expression products of the polynucleotide(s), and methods for using the expression products in assays or to generate antibodies useful in assays, as well as to methods for making the, polynucleotide(s), vectors, compositions vaccines, assays, inter alia.

The present invention thus relates to means for preventing and/or combating diseases caused by the WNV.

The invention relates to such immunogenic and vaccine compositions suitable for use in different animal (target or host) species susceptible to disease caused by WNV, including mammals, reptiles, birds, especially humans, companion mammals or animals such as canines, felines, equines, zoo mammals or animals, such as aquatic mammals e.g. seals, felines, equines, zoo reptiles such as snakes, crocodiles, aligators, and avian species, such as domesticated birds that are pets or poultry, or wild birds, e.g., canaries, parakeets, chickens, ducks, geese, turkeys, sparrows, crows, and the like.

The invention further relates to immunization and vaccination methods involving the immunogenic and vaccine compositions, for the target or host species. And on this aspect of the invention, mention is made that as to wild or non-domesticated animals, such as wild or non-domesticated birds or mammals (e.g., raccoons, squirrels, mice, or more generally rodents, felines, canines, etc.) compositions comprising one or more vectors that express one or more WNV epitopes or antigens or immunogens can be delivered via food, e.g., a bait drop, or mammal or bird food, left for consumption by wild or non-domesticated birds or mammals, that includes or contains the one or more vectors, so there may be administration thereof orally by the mammal or bird consuming the food. This route of administration may be advantageous when the one or more vectors is one or more poxviruses, e.g., an avipox virus such as an attenuated canarypox virus, for instance ALVAC, or an attenuated fowlpox virus, for instance TROVAC, or a vaccinia virus, such as an attenuated vaccinia virus, for instance NYVAC. Accordingly, the invention envisions oral or mucosal administration, as well as edible compositions that contain one or more of the inventive vectors, akin to the MERIAL rabies product RABORAL. From this disclosure and the knowledge in the art, the skilled artisan can formulate edible animal feed for a bird or mammal that contains a suitable dose of one or more inventive vectors. Furthermore, the invention comprehends topical administration of compositions containing vectors, see, e.g., U.S. Pat. No. 6,348,450 regarding topical administration of vector compositions, and devices for topical administration of compositions to wild or non-domesticated animals, see, e.g., WO01/95715, U.S. application Ser. No. 10/374,627, filed Feb. 26, 2003, for such devices for rodents and birds; each of which, together with each document cited or referenced therein, as with each document cited herein and each document referenced or cited in each document cited herein, is hereby incorporated herein by reference.

The invention further relates to means and methods that make differential diagnosis possible, e.g., methods that make it possible to make, or allow for, a distinction between an animal infected by the West Nile (WN) pathogenic virus and an animal administered a vaccine or immunogenic composition according to the invention.

In certain embodiments, the invention provides in vitro and/or in vivo expression vectors comprising a polynucleotide encoding the envelope protein E of WNV. In addition to the sources otherwise set forth herein for nucleic acid molecules encoding WNV E, mention is made of WO 02/072036, published Sep. 19, 2002, with claims of priority to U.S. Provisional applications Ser. Nos. 60/281,947 and 60/275,025, filed Apr. 5, 2001 and Mar. 12, 2001, respectively. These vectors advantageously also comprise the elements for the expression of the polynucleotide in a host cell.

In addition to the polynucleotide encoding E, the expression vectors according to the invention can comprise one or more other polynucleotides encoding other proteins of the WN virus, preferably structural proteins of the WN virus and said sequences are preferably chosen from among those encoding the pre-membrane protein prM and the membrane protein M.

The vector preferably comprises a polynucleotide forming a single encoding frame or coding region corresponding e.g. to prM-E, M-E, or advantageously prM-M-E, or epitopes thereof; that is, expression of a polyprotein or epitopes thereof are considered advantageous. A vector comprising several separate polynucleotides encoding the different proteins (e.g. prM and/or M and E or epitopes thereof) also falls within the scope of the present invention. The vector, especially for in vivo expression, can also comprise polynucleotides corresponding to more than one WN virus strain or isolate, for instance, two or more polynucleotides encoding E or prM-M-E, or epitope(s) thereof, of different strains.

Likewise, an immunogenic or vaccine composition can comprise one or more vectors for expression of polynucleotides corresponding to more than one WN virus strain or isolate, for instance, two or more polynucleotides encoding E or prM-M-E, or epitope(s) thereof, of different strains. The vector, especially for in vivo expression, can additionally comprise one or more nucleotide sequences encoding immunogens of other pathogenic agents and/or cytokines.

According to a preferred embodiment of the invention, the expression vector comprises a polynucleotide encoding prM-M-E and preferably in a single reading frame. In this regard, and particularly in regard to the herein preference for E, prM, M and combinations thereof in view of this disclosure also acknowledging other WNV proteins, it is noted that in this disclosure and particularly in the claims, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention. It is further noted that in combinations or polyproteins, it is advantageous that E be among the structural proteins of the combination or polyprotein.

The term polynucleotide encoding a protein of the WN virus primarily means a DNA fragment or isolated DNA molecule encoding said protein, or the complementary strand thereto; but, RNA is not excluded, as it is understood in the art that thymidine (T) in a DNA sequence is considered equal to uracil (U) in an RNA sequence. Thus, RNA sequences for use in the invention, e.g., for use in RNA vectors, can be derived from DNA sequences, by thymidine (T) in the DNA sequence being considered equal to uracil (U) in RNA sequences.

The term protein includes peptides and polypeptides. A protein fragment is immunologically active in the sense that once administered to the host, it is able to evoke an immune response of the humoral and/or cellular type directed against the protein. Preferably the protein fragment is such that it has substantially the same immunological activity as the total protein. Thus, a protein fragment according to the invention comprises or consists essentially of or consists of at least one epitope or antigenic determinant. The term epitope relates to a protein site able to induce an immune reaction of the humoral type (B cells) and/or cellular type (T cells).

Accordingly, a minimum structure of the polynucleotide is that it comprises or consists essentially of or consists of nucleotides to encode an epitope or antigenic determinant of the WNV protein or polyprotein. A polynucleotide encoding a fragment of the total protein or polyprotein, more advantageously, comprises or consists essentially of or consists of a minimum of 21 nucleotides, advantageously at least 42 nucleotides, and preferably at least 57, 87 or 150 consecutive or contiguous nucleotides of the sequence encoding the total protein or polyprotein. As mentioned earlier, epitope determination procedures, such as, generating overlapping peptide libraries (Hemmer B. et al., Immunology Today, 1998, 19 (4), 163-168), Pepscan (Geysen H. M. et al., Proc. Nat. Acad. Sci. USA, 1984, 81 (13), 3998-4002; Geysen H. M. et al., Proc. Nat. Acad. Sci. USA, 1985, 82 (1), 178-182; Van der Zee R. et al., Eur. J. Immunol., 1989, 19 (1), 43-47; Geysen H. M., Southeast Asian J. Trop. Med. Public Health, 1990, 21 (4), 523-533; Multipin® Peptide Synthesis Kits de Chiron) and algorithms (De Groot A. et al., Nature Biotechnology, 1999, 17, 533-561), can be used in the practice of the invention, without undue experimentation. Other documents cited and incorporated herein may also be consulted for methods for determining epitopes of an immunogen or antigen and thus nucleic acid molecules that encode such epitopes.

In an advantageous embodiment, the polynucleotides according to the invention comprise or consist essentially of or consist of the nucleotide sequence encoding one or two transmembrane domains and preferably two of them, located in the terminal part C of the E protein of WNV. For the WNV NY99 strain, these domains correspond to amino acid sequences 742 to 766 and 770 to 791 of GenBank AF196835.

Elements for the expression of the polynucleotide or polynucleotides are advantageously present in an inventive vector. In minimum manner, this comprises, consists essentially of, or consists of an initiation codon (ATG), a stop codon and a promoter, and optionally also a polyadenylation sequence for certain vectors such as plasmid and certain viral vectors, e.g., viral vectors other than poxviruses. When the polynucleotide encodes a polyprotein fragment, e.g. prM-E, M-E, prM-M-E, advantageously, in the vector, an ATG is placed at 5′ of the reading frame and a stop codon is placed at 3′. Other elements for controlling expression may be present, such as enhancer sequences, stabilizing sequences and signal sequences permitting the secretion of the protein.

Methods for making and/or administering a vector or recombinants or plasmid for expression of gene products of genes of the invention either in vivo or in vitro can be any desired method, e.g., a method which is by or analogous to the methods disclosed in, or disclosed in documents cited in: U.S. Pat. Nos. 6,130,066, 5,494,807, 5,514,375, 5,744,140, 5,744,141, 5,756,103, 5,762,938, 5,766,599, 5,990,091, 6,004,777, 6,130,066, 6,497,883, 6,464,984, 6,451,770, 6,391,314, 6,387,376, 6,376,473, 6,368,603, 6,348,196, 6,306,400, 6,228,846, 6,221,362, 6,217,883, 6,207,166, 6,207,165, 6,159,477, 6,153,199, 6,090,393, 6,074,649, 6,045,803, 6,033,670, 6,485,729, 6,103,526, 6,224,882, 6,312,682, 6, 312,683, 6,348,450, 4,603,112; 4,769,330; 5,174,993; 5,505,941; 5,338,683; 5,494,807; 4,394,448; 4,722,848; 4,745,051; 4,769,331; 5,591,639; 5,589,466; 4,945,050; 5,677,178; 5,591,439; 5,552,143; and 5,580,859; U.S. patent application Ser. No. 920,197, filed Oct. 16,1986; WO 94/16716; WO 96/39491; W091/11525; WO 98/33510; WO 90/01543; EP 0 370 573; EP 265785; Paoletti (1996) Proc. Natl. Acad. Sci. USA 93:11349-11353; Moss (1996) Proc. Natl. Acad. Sci. USA 93:11341-11348; Richardson (Ed) (1995) Methods in Molecular Biology 39, “Baculovirus Expression Protocols,” Humana Press Inc.; Smith et al. (1983) Mol. Cell. Biol. 3:2156-2165; Pennock et al. (1984) Mol. Cell. Biol. 4:399-406; Roizman Proc. Natl. Acad. Sci. USA 93:11307-11312; Andreansky et al. Proc. Natl. Acad. Sci. USA 93:11313-11318; Robertson et al. Proc. Natl. Acad. Sci. USA 93:11334-11340; Frolov et al. Proc. Natl. Acad. Sci. USA 93:11371-11377; Kitson et al. (1991) J. Virol. 65:3068-3075; Grunhaus et al. (1992) Sem. Virol. 3:237-52; Ballay et al. (1993) EMBO J. 4:3861-65; Graham (1990) Tibtech 8:85-87; Prevec et al. J. Gen. Virol. 70.429-434; Felgner et al. (1994) J. Biol. Chem. 269:2550-2561; (1993) Science 259:174549; McClements et al. (1996) Proc. Natl. Acad. Sci. USA 93:11414-11420; Ju et al. (1998) Diabetologia 41:736-739; and Robinson et al. (1997) Sem. Immunol. 9:271. Thus, the vector in the invention can be any suitable recombinant virus or virus vector, such as a poxvirus (e.g., vaccinia virus, avipox virus, canarypox virus, fowlpox virus, raccoonpox virus, swinepox virus, etc.), adenovirus (e.g., canine adenovirus), herpesvirus, baculovirus, retrovirus, etc. (as in documents incorporated herein by reference); or the vector can be a plasmid. The herein cited and incorporated herein by reference documents, in addition to providing examples of vectors useful in the practice of the invention, can also provide sources for non-WNV proteins or epitopes thereof, e.g., non-WNV immunogens or epitopes thereof, cytokines, etc. to be expressed by vector or vectors in, or included in, multivalent or cocktail immunogenic compositions or vaccines of the invention.

The present invention also relates to preparations comprising vectors, such as expression vectors, e.g., vaccines or immunogenic compositions. The preparations can comprise, consist essentially of, or consist of one or more vectors, e.g., expression vectors, such as in vivo expression vectors, comprising, consisting essentially or consisting of (and advantageously expressing) one or more of the WNV polynucleotides encoding E, prM, M or combinations or polyproteins thereof, especially as above-mentioned (e.g., E, or E and prM, or E and M, or E and prM and M, or polyprotein E-prM-M, or polyprotein prM-E, or polyprotein M-E, or at least an epitope thereof); and, advantageously, the vector contains and expresses a polynucleotide that includes, consists essentially of, or consists of a coding region encoding WNV E, in a pharmaceutically or veterinarily acceptable carrier, excipient or vehicle. Thus, according to an embodiment of the invention, the other vector or vectors in the preparation comprises, consists essentially of or consists of a polynucleotide that encodes, and under appropriate circumstances the vector expresses one or more other proteins of the WN virus, e.g. prM, M, prM-M, or an epitope thereof.

According to another embodiment, the vector or vectors in the preparation comprise, or consist essentially of, or consist of polynucleotide(s) encoding one or more proteins or epitope(s) thereof of WNV, e.g., of one or more WN virus strains or isolates; and, advantageously, in a suitable host cell or under appropriate conditions, the vector or vectors have express of the polynucleotide(s). The inventive preparation advantageously comprises, consists essentially of, or consists of, at least two vectors comprising, consisting essentially of, or consisting of, and advantageously also expressing, preferably in vivo under appropriate conditions or suitable conditions or in a suitable host cell, polynucleotides from different WN strains or isolates encoding the same proteins and/or for different proteins, but preferably for the same proteins. As to preparations containing one or more vectors containing, consisting essentially of or consisting of polynucleotides encoding, and preferably expressing, advantageously in vivo, WNV E, or prM-M-E, or an epitope thereof, it is preferred that the expression products be from two, three or more different WN strains or isolates, advantageously strains. The invention is also directed at mixtures of vectors that contain, consist essentially of, or consist of coding for, and express, prM, M, E, prM-M, prM-E or M-E of different strains. It is preferred that in such mixtures, at least one vector contain, consist essentially of, or consist of, coding for, and express, E.

According to yet another embodiment and as will be shown in greater detail hereinafter, the other vector or vectors in the preparation comprise and express one or more cytokines and/or one or more immunogens of one or more other pathogenic agents. Sources for cytokines, immunogens for other pathogenic agents or epitope(s) thereof, and nucleic acid molecules encoding the same, may be found in herein cited documents, as well as in, WO02096349, WO0208162, WO0020025, WO00152888, WO0145735, WO00127097, WO0116330, WO0077210, WO0077188, WO0077043, WO9842743, WO9833928, WO9749826, WO9749825, U.S. Pat. Nos. 6,387,376, 6,306,400, 6,159,477, 6,156,567, 6,153,199, 6,090,393, 6,074,649, 6,033,670.

The invention also relates to various combinations of different embodiments herein disclosed, e.g., compositions or vaccines containing various vectors, compositions or vaccines containing a vector and a protein (WNV and/or non-WNV) and/or cytokine, etc.

The preparations comprising an in vitro or in vivo expression vector comprising and expressing a polynucleotide encoding prM-M-E constitute a preferred embodiment of the invention. According to another advantageous embodiment of the invention, the in vivo or in vitro expression vectors comprise as the sole polynucleotide or polynucleotides of the WN virus, a polynucleotide encoding the protein E, optionally associated with prM and/or M, preferably encoding prM-M-E and optionally a signal sequence of the WN virus. Thus, in advantageous embodiments the polynucleotide can additionally encode a signal sequence of WNV.

According to a further advantageous embodiment, one or more of the non-structural proteins NS2A, NS2B and NS3 are expressed jointly with the structural proteins according to the invention, either via the same expression vector, or via their own expression vector. They are preferably expressed together on the basis of a single polynucleotide, e.g., as a polyprotein. That is, in certain embodiments, the vector further contains, consists essentially of or consists of, one or more nucleotides encoding NS2A, NS2B and/or NS3, or a composition or vaccine further contains, consists essentially of or consists of one or more additional vectors that contains, consists essentially of or consists of, one or more nucleotides encoding NS2A, NS2B and/or NS3; this vector or these vectors advantageously express(es) the non-structural protein(s); and, NS2A, NS2B and NS3 are advantageously expressed jointly, and more advantageously, as a polyprotein.

Thus, the invention also relates to vector such as an in vivo or in vitro expression vector comprising, consisting essentially of or consisting of the polynucleotide(s) encoding NS2A, NS2B, NS3, combinations thereof, including polyproteins thereof, such as NS2A-NS2B-NS3. The vector can be one of the above-described vectors comprising, consisting essentially of or consisting of a polynucleotide encoding one or more structural proteins, e.g., E, prM, M, combinations and polyproteins thereof such as prM-E, M-E, or prM-M-E, e.g., such a vector that contains or consists essentially of polynucleotides encoding structural protein or proteins or epitopes thereof can also contain or consist essentially thereof polynucleotides encoding one or more non-structural proteins, combination thereof, polyproteins thereof, or epitopes thereof. As an alternative, the invention relates to a preparation as described hereinbefore, also incorporating at least one of the vectors that contain polynucleotide(s) encoding and advantageously expressing a non-structural protein and optionally a pharmaceutically or veterinarily acceptable carrier, vehicle or excipient.

For preparing vectors, e.g., expression vectors, according to the invention, the skilled artisan has available various strains of the WN virus and the description of the nucleotide sequence of their genome, see, e.g., discussion herein and Savage H. M. et al. (Am. J. Trop. Med. Hyg. 1999, 61 (4), 600-611), table 2, which refers to 24 WN virus strains and gives access references to polynucleotide sequences in GenBank, as well as other herein cited and incorporated by reference documents.

Reference is, for example, made to strain NY99 (GenBank AF196835). In GenBank, for each protein the corresponding DNA sequence is given (nucleotides 466-741 for prM, 742-966 for M, 967-2469 for E, or 466-2469 for prM-M-E, 3526-4218 for NS2A, 4219-4611 for NS2B and 4612-6468 for NS3, or 3526-6468 for NS2A-NS2B-NS3). By comparison and alignment of the sequences, the determination of a polynucleotide encoding such a protein in another WNV strain is readily determined.

As discussed herein, the term polynucleotide is understood to mean a nucleic acid sequence encoding a protein or a fragment thereof or an epitope thereof specific to a particular WN virus; and, by equivalence, the term polynucleotide is understood to include the corresponding nucleotide sequences of the different WN virus strains and nucleotide sequences differing by due to codon degeneracy. Thus, a polynucleotide encoding WNV E is understood as comprising, consisting essentially of or consisting of (a) nt 466-2469 of NY99 (GenBank AF196835), (b) corresponding sequences of different WNV strains, and (c) nucleotide sequences that encode WNV E but differ from (a) and (b) due to codon degeneracy.

Within the family of WN viruses, identity between amino acid sequences (“sequence identity”) prM-M-E relative to that of NY99 is equal to or greater than 90%. Thus, the invention covers polynucleotides encoding proteins having amino acid sequences, whose sequence identity or homology with the native WNV amino acid sequence for the protein is equal to or greater than 90%, advantageously 92%, preferably 95% and more specifically 98%. For instance, an expressed E protein can have greater than 90% identity with the sequence of the polypeptide expressed from (a) nt 466-2469 of NY99 (GenBank AF196835), (b) corresponding sequences of different WNV strains, and/or (c) nucleotide sequences that encode WNV E but differ from (a) and (b) due to codon degeneracy; advantageously at least 92%, more advantageously at least 95%, and even more advantageously at least 98%.

Therefore, the invention comprehends polynucleotides that express such homologous polypeptides; and the corresponding degrees of homology or identity of those polynucleotides to polynucleotides encoding polypeptides to which homologous polypeptides have homology or identity. Homologous polypeptides advantageously contain one or more epitopes of the polypeptide to which there is identity or homology, such that homologous polypeptides exhibit immunological similarity or identity to the polypeptide to which there is identity or homology, e.g., the homologous polyptide elicits similar or better immune response (to the skilled immunologist) than polypeptide to which there is identity or homology and/or the homologous polypeptide binds to antibodies elicited by and/or to which the polypeptide to which there is identity or homology binds, advantageously and not to other antibodies.

Accordingly, fragments of homologous polypeptides and of polypeptides to which there is identity or homology, advantageously those fragments which exhibit immunological similarity or identity to homologous polypeptides or polypeptides to which there is identity or homology, are envisoned as being expressed, and therefore, polynucleotides therefor which may represent fragments of polynucleotides of homologous polypeptides and of polypeptides to which there is identity or homology, are also envisioned by and useful in the instant invention.

The term “sequence identity” indicates a quantitative measure of the degree of homology between two amino acid sequences of equal length or between two nucleotide sequences of equal length. If the two sequences to be compared are not of equal length, they must be aligned to best possible fit possible with the insertion of gaps or alternatively, truncation at the ends of the protein sequences. The sequence identity can be calculated as ((N_{ref}−N_{dif})/N_{ref})×100, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{dif}=2 and N_{ref}=8). A gap is counted as non-identity of the specific residue(s), i.e. the DNA sequence AGTGTC will have a sequence identity of 75% with the DNA sequence AGTCAGTC (N_{dif}=2 and N_{ref}=8). Sequence identity can alternatively be calculated by the BLAST program e.g. the BLASTP program (Pearson W. R and D. J. Lipman (1988) PNAS USA 85:2444-2448) (www.ncbi.nlm.nih.gov/cgi-bin/BLAST). In one aspect of the invention, alignment is performed with the sequence alignment method ClustalW with default parameters as described by Thompson J., et al 1994, available at http://www2.ebi.ac.uk/clustalw/. Thus, a polynucleotide can be any nucleic acid molecule including DNA, RNA, LNA (locked nucleic acids), PNA, RNA, dsRNA, RNA-DNA-hybrid, and non-naturally occurring nucleosides.

And from the herein disclosure, advantageously, proteins or polypeptides expressed by vectors of the invention are immunologically active peptides and polypeptides, e.g., with respect to polypeptides or proteins of NY99, proteins or polypeptides expressed by vectors of the invention can be:
a) corresponding proteins or polypeptides of one or more different WN virus strains or isolates,
b) proteins differing therefrom (from NY99 and/or a)), but maintaining with a native WN protein an identity equal to or greater than 90%, advantageously greater than or equal to 92%, more advantageously greater than or equal to 95% and even more advantageously greater than or equal to 98%.
   Thus, a reference to a WNV protein may involve additional proteins as herein discussed.

Different WN virus strains are accessible in collections, especially in the American Type Culture Collection (ATCC), e.g. under access numbers VR-82 or VR-1267, and as otherwise herein discussed, with it noted that the Kunjin virus is considered to be a WN virus.

In the invention, preferably the polynucleotide also comprises a nucleotide sequence encoding a signal peptide, located upstream of the coding for the expressed protein to facilitate the secretion thereof; and accordingly, the invention comprehends the expression of a WNV polypeptide, such as a WNV antigen, immunogen, or fragment thereof, e.g., epitope, with a leader or signal sequence. The leader or signal sequence can be an endogenous sequence, e.g. the natural signal sequence of a WNV polypeptide, which can be from the same WN virus strain or isolate or another strain or isolate. For example, for the NY99 WN virus, the endogenous signal sequence for E is encoded at nucleotides 922 to 966 of the GenBank sequence and for prM it is encoded at nucleotides 421 to 465. The leader or signal sequence can also be a heterologous sequence, and thus encoded by a nucleotide sequence that is heterologous to WNV. For example, the leader or signal sequence can be endogenous to the vector, or a leader or signal sequence that is heterologous to both the vector and WNV, such as a signal peptide of tissue plasminogen activator (tPA), e.g., human tPA, and thus, the vector or the polynucleotide therein can include a sequence encoding the leader or signal peptide, e.g., the leader or signal peptide of human tissue plasminogen activator (tPA) (Hartikka J. et al., Human Gene Therapy, 1996, 7, 1205-1217). The nucleotide sequence encoding the signal peptide is advantageiously inserted in frame and upstream of the sequence encoding the WNV polypeptide, e.g., E or its combinations, e.g. prM-M-E, M-E, prM-E.

According to an embodiment of the invention, the vectors, e.g., in vivo expression vectors, are viral vectors.

Viral vectors, e.g., viral expression vectors are advantageously: poxviruses, e.g. vaccinia virus or an attenuated vaccinia virus, (for instance, MVA, a modified Ankara strain obtained after more than 570 passages of the Ankara vaccine strain on chicken embryo fibroblasts; see Stickl H. and Hochstein-Mintzel V., Munch. Med. Wschr., 1971, 113, 1149-1153; Sutter G. et al., Proc. Natl. Acad. Sci. U.S.A., 1992, 89, 10847-10851; available as ATCC VR-1508; or NYVAC, see U.S. Pat. No. 5,494,807, for instance, Examples 1 to 6 and et seq of U.S. Pat. No. 5,494,807 which discuss the construction of NYVAC, as well as variations of NYVAC with additional ORFs deleted from the Copenhagen strain vaccinia virus genome, as well as the insertion of heterologous coding nucleic acid molecules into sites of this recombinant, and also, the use of matched promoters; see also WO96/40241), avipox virus or an attenuated avipox virus (e.g., canarypox, fowlpox, dovepox, pigeonpox, quailpox, ALVAC or TROVAC; see, e.g. U.S. Pat. Nos. 5,505,941, 5,494,807), swinepox, raccoonpox, camelpox, or myxomatosis virus; adenoviruses, such as avian, canine, porcine, bovine, human adenoviruses; or herpes viruses, such as equine herpes virus (EHV serotypes 1 and 4), canine herpes virus (CHV), feline herpes virus (FHV), bovine herpes viruses (BHV serotypes 1 and 4), porcine herpes virus (PRV), Marek's disease virus (MDV serotypes 1 and 2), turkey herpes virus (HVT or MDV serotype 3), or duck herpes virus. When a herpes virus is used, the vector HVT is preferred for the vaccination of the avian species and the vector EHV for the vaccination of horses.

More generally in certain embodiments, it may be advantageous to match a vector to a host, such as an equine virus, e.g., EHV to use in equines, or a vector that is an avian pathogen, such as fowlpox HVT, MDV or duck herpes to use in avians such as poultry or chickens, or a vector that is a bovine pathogen such as BHV to use in bovines such as cows, or a vector that is a porcine pathogen such a porcine herpes virus to use in porcines, or a vector that is a canine pathogen such as canine adenovirus or canine herpes virus to use in canines such as dogs, a vector that is a feline pathogen such as FHV to use in felines, as this may allow for an immune response against the vector and thus provide an immune response against a pathogen of the host or target species in addition to an immune response against WNV.

However, it is also noted that it can be advantageous that the vector not be a natural pathogen of the host; for instance, so that the vector can have expression of the exogenous, e.g., WNV coding sequences, but with limited or no replication; for example, the use of an avipox vector in a mammalian host, as in U.S. Pat. No. 5,174,993. It is also noted that the invention comprehends vaccines, immunological and immunogenic compositions, with those terms being used in the sense attributed to them in the art; see, e.g., documents cited herein, such as U.S. Pat. No. 6,497,883.

According to another embodiment of the invention, the poxvirus vector, e.g., expression vector, is a canarypox virus or a fowlpox virus vector, advantageously an attenuated canarypox virus or fowlpox virus. In this regard, is made to the canarypox available from the ATCC under access number VR-111. Attenuated canarypox viruses are described in U.S. Pat. No. 5,756,103 (ALVAC) and WO01/05934. Numerous fowlpox virus vaccination strains are also available, e.g. the DIFTOSEC CT strain marketed by MERIAL and the NOBILIS VARIOLE vaccine marketed by Intervet; and, reference is also made to U.S. Pat. No. 5,766,599 which pertains to the atenuated fowlpox strain TROVAC.

For information on poxviruses and how to generate recombinants thereof and how to administer recombinants thereof, the skilled artisan can refer documents cited herein and to WO90/12882, e.g., as to vaccinia virus mention is made of U.S. Pat. Nos. 4,769,330, 4,722,848, 4,603,112, 5,110,587, 5,494,807, and 5,762,938 inter alia; as to fowlpox, mention is made of U.S. Pat. Nos. 5,174,993, 5,505,941 and U.S. Pat. No. 5,766,599 inter alia; as to canarypox mentionis made of U.S. Pat. No. 5,756,103 inter alia; as to swinepox mention is made of U.S. Pat. No. 5,382,425 inter alia; and, as to raccoonpox, mention is made of WO00/03030 inter alia.

When the expression vector is a vaccinia virus, insertion site or sites for the polynucleotide or polynucleotides to be expressed are advantageously at the thymidine kinase (TK) gene or insertion site, the hemagglutinin (HA) gene or insertion site, the region encoding the inclusion body of the A type (ATI); see also documents cited herein, especially those pertaining to vaccinia virus. In the case of canarypox, advantageously the insertion site or sites are ORF(s) C3, C5 and/or C6; see also documents cited herein, especially those pertaining to canarypox virus. In the case of fowlpox, advantageously the insertion site or sites are ORFs F7 and/or F8; see also documents cited herein, especially those pertaining to fowlpox virus. The insertion site or sites for MVA virus area advantageously as in various publications, including Carroll M. W. et al., Vaccine, 1997, 15 (4), 387-394; Stittelaar K. J. et al., J. Virol., 2000, 74 (9), 4236-4243; Sutter G. et al., 1994, Vaccine, 12 (11), 1032-1040; and, in this regard it is also noted that the complete MVA genome is described in Antoine G., Virology, 1998, 244, 365-396, which enables the skilled artisan to use other insertion sites or other promoters.

Preferably, when the expression vector is a poxvirus, the polynucleotide to be expressed is inserted under the control of a specific poxvirus promoter, e.g., the vaccinia promoter 7.5 kDa (Cochran et al., J. Virology, 1985, 54, 30-35), the vaccinia promoter 13L (Riviere et al., J. Virology, 1992, 66, 3424-3434), the vaccinia promoter HA (Shida, Virology, 1986, 150, 451-457), the cowpox promoter ATI (Funahashi et al., J. Gen. Virol., 1988, 69, 35-47), the vaccinia promoter H6 (Taylor J. et al., Vaccine, 1988, 6, 504-508; Guo P. et al. J. Virol., 1989, 63, 4189-4198; Perkus M. et al., J. Virol., 1989, 63, 3829-3836), inter alia.

Preferably, for the vaccination of mammals the expression vector is a canarypox or a fowlpox. In this way, there can be expression of the heterologous proteins, e.g., WNV proteins, with limited or no productive replication. Preferably, for the vaccination of avians, e.g., chickens, ducks, turkeys and geese, the expression vector is a canarypox or a fowlpox.

When the expression vector is a herpes virus of turkeys or HVT, advantageous insertion site or sites are located in the BamHI I fragment or in the BamHI M fragment of HVT. The HVT BamHI I restriction fragment comprises several open reading frames (ORFs) and three intergene regions and comprises several preferred insertion zones, such as the three intergene regions 1, 2 and 3, which are preferred regions, and ORF UL55 (see, e.g., FR-A-2 728 795, U.S. Pat. No. 5,980,906). The HVT BamiHI M restriction fragment comprises ORF UL43, which is also a preferred insertion site (see, e.g., FR-A-2 728 794, U.S. Pat. No. 5,733,554).

When the expression vector is an EHV-1 or EHV-4 herpes virus, advantageous insertion site or sites include TK, UL43 and UL45 (see, e.g., EP-A-668355).

Preferably, when the expression vector is a herpes virus, the polynucleotide to be expressed is inserted under the control of a eukaryotic promoter, such as a strong eukaryote promoter, preferably a CMV-IE (murine or human) promoter; that is, in embodiments herein, the polynucleotide to be expressed is operably linked to a promoter, and in herpes virus embodiments, advantageously the polynucleotide to be expressed is operably linked to a strong eukatyotic promoter such as a mCMV-IE or hCMV-IE promoter. Strong promoters are also discussed herein in relation to plasmids as vectors.

According to a yet further embodiment of the invention, the vector, e.g., in vivo expression vector, is a plasmidic vectors, also known as a plasmid vector or a DNA plasmid vector, e.g., the type of plasmid vector employed in that which is known as a DNA vaccine (in contrast with a transfection plasmid used in homologous recombination to generate a recombinant virus, which is not used in a DNA vaccine).

The term plasmid covers any DNA transcription unit in the form of a polynucleotide sequence comprising a polynucleotide according to the invention and the elements necessary for its in vivo expression of that which is encoded by the polynucleotide in a cell or cells of the desired host or target; and, in this regard, it is noted that there is a supercoiled or non-supercoiled, circular plasmid, as well as linear forms, all of which are intended to be within the scope of the invention.

Each plasmid comprises or contains or consists essentially of, in addition to the polynucleotdie encoding the antigen or epitope of the pathogen or pathogens, e.g., WNV (or WNV and another pathogen), a promoter for expression, in the host cells cor cells, of the polynucleotide; and, the polynucleotide may be said to be operably linked to the promoter or under the control of the promoter or dependent upon the promoter. In general, it is advantageous to employ a eukaryotic promoter, e.g., a strong eukaryotic promoter. The preferred strong eukaryote promoter is the early cytomegalovirus promoter (CMV-IE) of human or murine origin, or optionally having another origin such as the rat or guinea pig. The CMV-IE promoter can comprise the actual promoter part, which may or may not be associated with the enhancer part. Reference can be made to EP-A-260 148, EP-A-323 597, U.S. Pat. Nos. 5,168,062, 5,385,839, and 4,968,615, as well as to PCT WO87/03905. The CMV-IE promoter is preferably a human CMV-IE (Boshart M. et al., Cell., 1985, 41, 521-530) or murine CMV-IE.

In more general terms, the promoter has either a viral or a cellular origin. A strong viral promoter other than CMV-IE that may be usefully employed in the practice of the invention is the early/late promoter of the SV40 virus or the LTR promoter of the Rous sarcoma virus. A strong cellular promoter that may be usefully employed in the practice of the invention is the promoter of a gene of the cytoskeleton, such as e.g. the desmin promoter (Kwissa M. et al., Vaccine, 2000, 18 (22), 2337-2344), or the actin promoter (Miyazaki J. et al., Gene, 1989, 79 (2), 269-277).

Functional subfragments of these promoters, i.e., portions of these promoters that maintain an adequate promoting activity, are included within the present invention, e.g. truncated CMV-IE promoters according to WO98/00166 or U.S. Pat. No. 6,156,567 can be used in the practice of the invention. A promoter in the practice of the invention consequently includes derivatives and subfragments of a full-length promoter that maintain an adequate promoting activity and hence function as a promoter, preferably promoting activity substantially similar to that of the actual or full-length promoter from which the derivative or subfragment is derived, e.g., akin to the activity of the truncated CMV-IE promoters of U.S. Pat. No. 6,156,567 to the activity of full-length CMV-IE promoters. Thus, a CMV-IE promoter in the practice of the invention can comprise or consist essentially of or consist of the promoter portion of the full-length promoter and/or the enhancer portion of the full-length promoter, as well as derivatives and subfragments.

Preferably, the plasmids comprise or consist essentially of other expression control elements. It is particularly advantageous to incorporate stabilizing sequence(s), e.g., intron sequence(s), preferably intron II of the rabbit P-globin gene (van Ooyen et al., Science, 1979, 206: 337-344).

As to the polyadenylation signal (polyA) for the plasmids and viral vectors other than poxviruses, use can more be made of the polyA signal of the bovine growth hormone (bGH) gene (see U.S. Pat. No. 5,122,458), or the poly(A) signal of the rabbit β-globin gene or the poly(A) signal of the SV40 virus.

As to other expression control elements usable in plasmids, attention is directed to expression control elements that are useful in herpes virus expression vectors.

According to another embodiment of the invention, the expression vectors are expression vectors used for the in vitro expression of proteins in an appropriate cell system. The expressed proteins can be harvested in or from the culture supernatant after, or not after secretion (if there is no secretion a cell lysis typically occurs or is performed), optionally concentrated by concentration methods such as ultrafiltration and/or purified by purification means, such as affinity, ion exchange or gel filtration-type chromatography methods.

Protein production can take place by the transfection of mammalian cells by plasmids, by replication or expression without productive replication of viral vectors on mammal cells or avian cells, or by Baculovirus replication (see, e.g., U.S. Pat. No. 4,745,051; Vialard J. et al., J. Virol., 1990 64 (1), 37-50; Verne A., Virology, 1988, 167, 56-71), e.g. Autographa californica Nuclear Polyhedrosis Virus AcNPV, on insect cells (e.g. Sf9 Spodoptera frugiperda cells, ATCC CRL 1711; see also U.S. Pat. Nos. 6,228,846, 6,103,526). Mammalian cells which can be used are advantageously hamster cells (e.g. CHO or BHK-21) or-monkey cells (e.g. COS or VERO). Thus, the invention accordingly comprhends expression vectors incorporating a polynucleotide according to the invention, as well as the thus produced or expressed WNV proteins or fragments thereof from in vitro expression, and the preparations containing the same.

Accordingly, the present invention also relates to WNV protein-concentrated and/or purified preparations. When the polynucleotide encodes several proteins, they are cleaved, and the aforementioned preparations then contain cleaved proteins.

The present invention also relates to immunogenic compositions and vaccines against the WN virus comprising at least one in vivo expression vector according to the invention and a pharmaceutically or veterinarily acceptable excipient or carrier or vehicle, and optionally an adjuvant.

An immunogenic composition covers any composition which, once administered to the target species, induces an immune response against the WN virus. The term vaccine is understood to mean a composition able to induce an effective protection. The target species include mammals, e.g., equines, canines, felines, bovines, porcines and humans; reptiles, and birds or avians; preferably horse, dog, cat, pig, alligator; and, in the case of birds or avians, geese, turkeys, chickens and ducks. This list is meant to include reproducing animals, egg-laying animals, meat-producing animals or production animals (animals whose flesh is commonly consumed by some humans), and companion animals (animals who are kept as pets by humans).

The pharmaceutically or veterinarily acceptable carriers or vehicles or excipients are well known to the one skilled in the art. For example, a pharmaceutically or veterinarily acceptable carrier or vehicle or excipient can be a 0.9% NaCl saline solution or a phosphate buffer. The pharmaceutically or veterinarily acceptable carrier or vehicle or excipients may be any compound or combination of compounds facilitating the administration of the vector (or protein expressed from an inventive vector in vitro); advantageously, the carrier, vehicle or excipient may facilitate transfection and/or improve preservation of the vector (or protein). Doses and dose volumes are herein discussed in the general description of immunization and vaccination methods, and can also be determined by the skilled artisan from this disclosure read in conjunction with the knowledge in the art, without any undue experimentation.

The immunogenic compositions and vaccines according to the invention preferably comprise or consist essentially of one or more adjuvants. Particularly suitable adjuvants for use in the practice of the present invention are (1) polymers of acrylic or methacrylic acid, maleic anhydride and alkenyl derivative polymers, (2) immunostimulating sequences (ISS), such as oligodeoxyribonucleotide sequences having one ore more non-methylated CpG units (Klinrnan D. M. et al., Proc. Natl. Acad. Sci., USA, 1996, 93, 2879-2883; W098/16247), (3) an oil in water emulsion, such as the SPT emulsion described on p 147 of “Vaccine Design, The Subunit and Adjuvant Approach” published by M. Powell, M. Newman, Plenum Press 1995, and the emulsion MF59 described on p 183 of the same work, (4) cation lipids containing a quaternary ammonium salt, (5) cytokines, (6) aluminum hydroxide or aluminum phosphate or (7) other adjuvants discussed in any document cited and incorporated by referenc into the instant application, or (8) any combinations or mixtures thereof.

The oil in water emulsion (3), which is especially appropriate for viral vectors, can be based on:
light liquid paraffin oil (European pharmacopoeia type),
isoprenoid oil such as squalane, squalene,
oil resulting from the oligomerization of alkenes, e.g. isobutene or decene,
esters of acids or alcohols having a straight-chain alkyl group, such as vegetable oils, ethyl oleate, propylene glycol, di(caprylate/caprate), glycerol tri(caprylate/caprate) and propylene glycol dioleate, or
esters of branched, fatty alcohols or acids, especially isostearic acid esters.

The oil is used in combination with emulsifiers to form an emulsion. The emulsifiers may be nonionic surfactants, such as:
esters of on the one hand sorbitan, mannide (e.g. anhydromannitol oleate), glycerol, polyglycerol or propylene glycol and on the other hand oleic, isostearic, ricinoleic or hydroxystearic acids, said esters being optionally ethoxylated,
polyoxypropylene-polyoxyethylene copolymer blocks, such as Pluronic, e.g., L121.

Among the type (1) adjuvant polymers, preference is given to polymers of crosslinked acrylic or methacrylic acid, especially crosslinked by polyalkenyl ethers of sugars or polyalcohols. These compounds are known under the name carbomer (Pharmeuropa, vol. 8, no. 2, June 1996). One skilled in the art can also refer to U.S. Pat. No. 2,909,462, which provides such acrylic polymers crosslinked by a polyhydroxyl compound having at least three hydroxyl groups, preferably no more than eight such groups, the hydrogen atoms of at least three hydroxyl groups being replaced by unsaturated, aliphatic radicals having at least two carbon atoms. The preferred radicals are those containing 2 to 4 carbon atoms, e.g. vinyls, allyls and other ethylenically unsaturated groups. The unsaturated radicals can also contain other substituents, such as methyl. Products sold under the name Carbopol (BF Goodrich, Ohio, USA) are especially suitable. They are crosslinked by allyl saccharose or by allyl pentaerythritol. Among them, reference is made to Carbopol 974P, 934P and 971P.

As to the maleic anhydride-alkenyl derivative copolymers, preference is given to EMA (Monsanto), which are straight-chain or crosslinked ethylene-maleic anhydride copolymers and they are, for example, crosslinked by divinyl ether. Reference is also made to J. Fields et al., Nature 186: 778-780, Jun. 4, 1960.

With regard to structure, the acrylic or methacrylic acid polymers and EMA are preferably formed by basic units having the following formula: 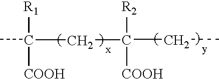
in which:
R₁ and R₂, which can be the same or different, represent H or CH₃
x=0 or 1, preferably x=1
y=1 or 2,with x+y=2.

For EMA, x=0 and y=2 and for carbomers x=y=1.

These polymers are soluble in water or physiological salt solution (20 g/l NaCl) and the pH can be adjusted to 7.3 to 7.4, e.g., by soda (NaOH), to provide the adjuvant solution in which the expression vector(s) can be incorporated. The polymer concentration in the final vaccine composition can range between 0.01 and 1.5% w/v, advantageously 0.05 to 1% w/v and preferably 0.1 to 0.4% w/v.

The cationic lipids (4) containing a quaternary ammonium salt which are advantageously but not exclusively suitable for plasmids, are preferably those having the following formula: 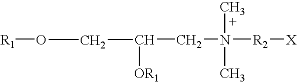
in which R₁ is a saturated or unsaturated straight-chain aliphatic radical having 12 to 18 carbon atoms, R₂ is another aliphatic radical containing 2 or 3 carbon atoms and X is an amine or hydroxyl group.

Among these cationic lipids, preference is given to DMRIE (N-(2-hydroxyethyl)-N,N-dimethyl-2,3-bis(tetradecyloxy)-1-propane ammonium; WO96/34109), preferably associated with a neutral lipid, preferably DOPE (dioleoyl-phosphatidyl-ethanol amine; Behr J. P., 1994, Bioconjugate Chemistry, 5, 382-389), to form DMRIE-DOPE.

Preferably, the plasmid mixture with the adjuvant is formed extemporaneously and preferably contemporaneously with administration of the preparation or shortly before administration of the preparation; for instance, shortly before or prior to administration, the plasmid-adjuvant mixture is formed, advantageously so as to give enough time prior to administration for the mixture to form a complex, e.g. between about 10 and about 60 minutes prior to administration, such as approximately 30 minutes prior to administration.

When DOPE is present, the DMRIE:DOPE molar ratio is preferably about 95: about 5 to about 5:about 95, more preferably about 1: about 1, e.g., 1:1.

The DMRIE or DMRIE-DOPE adjuvant:plasmid weight ratio can be between about 50: about 1 and about 1: about 10, such as about 10: about 1 and about 1 :about 5, and preferably about 1: about 1 and about 1: about 2, e.g., 1:1 and 1:2.

The cytokine or cytokines (5) can be in protein form in the immunogenic or vaccine composition, or can be co-expressed in the host with the immunogen or immunogens or epitope(s) thereof. Preference is given to the co-expression of the cytokine or cytokines, either by the same vector as that expressing the immunogen or immunogens or epitope(s) thereof, or by a separate vector therefor.

The cytokine(s) can be chosen from: interleukin 18 (IL-18), interleukin 12 (IL-12), interleukin 15 (IL-15), MIP-1α (macrophage inflammatory protein 1α; Marshall E. et al., Br. J. Cancer, 1997, 75 (12), 1715-1720), GM-CSF (Granulocyte-Macrophage Colony-Stimulating Factor). Particular reference is made to avian cytokines, for instance, those of the chicken, such as cIL-18 (Schneider K. et al., J. Interferon Cytokine Res., 2000, 20 (10), 879-883), cIL-15 (Xin K.-Q. et al., Vaccine, 1999, 17, 858-866), and equine cytokines, for instance equine GM-CSF (WO00/77210). Preferably, use is made of cytokines of the species to be vaccinated; that is, advantageously, the cytokine is matched to the target or host species, and, note for example, canine GM-CSF (example 8 of WO00/77043), feline GM-CSF (example 9 of WO00/77043).

WO00/77210 provides the nucleotide sequence and the amino acid sequence corresponding to equine GM-CSF, the in vitro GM-CSF production and the construction of vectors (e.g., plasmids and viral vectors) permitting in vivo equine GM-CSF expression. These proteins, plasmids and viral vectors can be used in immunogenic compositions and equine vaccines according to the invention. For example, use can be made of the plasmid pJP097 described in example 3 of WO00/77210 or use can be made of the teaching of the latter in order to produce other vectors or for the in vitro production of equine GM-CSF and the incorporation of the vectors or the equine GM-CSF into immunogenic compositions or equine vaccines according to the invention.

The present invention also relates to immunogenic compositions and so-called subunit vaccines, incorporating or comprising or consisting essentially of the protein E and optionally one or more other herein mentioned proteins of the WN virus, e.g., prM or M and advantageously produced by in vitro expression in the manner described herein, as well as a pharmaceutically or veterinarily acceptable carrier or vehicle or excipient.

The pharmaceutically or veterinarily acceptable carrier or vehicle or excipient can be determined by the skilled artisan without undue experimentation from the disclosure herein and the knowledge in the art, e.g., by reference to documents cited and incorporated herein or documents referenced in herein cited documents and incorporated herein by reference; and, can for example, be 0.9% NaCl saline solution or phosphate buffer.

The immunogenic compositions and subunit vaccines according to the invention preferably comprise or consist essentially of one or more adjuvants. Especially suitable for use in the present invention are (1) an acrylic or methacrylic acid polymer, or a maleic anhydride and alkenyl derivative polymer, (2) an immunostimulating sequence (ISS), such as an oligodeoxyribonucleotide sequence having one or more non-methylated CpG units (Klinman D. M. et al., Proc. Natl. Acad. Sci. USA, 1996, 93, 2879-2883; WO98/16247), (3) an oil in water emulsion, such as the emulsion SPT described on p 147 of “Vaccine Design, The Subunit and Adjuvant Approach”, published by M. Powell, M. Newmann, Plenum Press 1995, and the emulsion MF59 described on p 183 of the same work, (4) a water in oil emulsion (EP-A-639 071), (5) saponin, such as Quil-A, or (6) alumina hydroxide or an equivalent. The different types of adjuvants defined under 1), 2) and 3) have been described in greater detail herein in connection with the expression vector-based vaccines and immunogenic compositions.

The doses and dose volumes are discussed herein in connection with the general description of immunization and vaccination methods.

Animals immunized with immunogenic compositions or vaccines according to the invention develop a specific immunity against WNV, which during a WNV infection involves a decrease of the viremia, and indeed can totally block the virus, as compared with unvaccinated control animals. This advantageous aspect of the invention may be used to stop the transmission of the WN virus, to limit the existence of viral reservoirs and to prevent outbreaks of West Nile disease, notably in human.

Another advantageous aspect of the invention is that protective immunity can be transmitted from vaccinated subjects to the offspring.

According to the invention, the vaccination against the WN virus can be combined with other vaccinations within the framework of vaccination programs, in the form of immunization or vaccination kits or methods, or in the form of multivalent immunogenic compositions and multivalent vaccines, i.e. comprising or consisting essentially of at least one vaccine component against the WN virus and at least one vaccine component against at least one other pathogenic agent. This also includes the expression by the same expression vector of genes of at least two pathogenic agents, including the WN virus.

The invention thus also relates to a multivalent or “cocktail” immunogenic composition or a multivalent or “cocktail” vaccine against the WN virus and against at least one other pathogen of the target species, using the same in vivo expression vector containing and expressing at least one polynucleotide of the WN virus according to the invention and at least one polynucleotide expressing an immunogen of another pathogen. As to combination or multivalent or “cocktail” immunogenic compositions or vaccines, as well as to immunogens or antigens or epitopes thereof to be in or expressed by such compositions or vaccines, attention is directed to herein cited and incorporated by reference documents, as well as to U.S. Pat. Nos. 5,843,456 and 6,368,603.

The “immunogen” expressed by a vector of the invention or used in multivalent or “cocktail” compositions or vaccines is understood to mean a protein, glycoprotein, polypeptide, peptide, epitope or derivative, e.g. fusion protein, inducing an immune response, preferably of a protective nature.

As discussed herein, these multivalent compositions or vaccines can also comprise or consist essentially of a pharmaceutically or veterinarily acceptable carrier or vehicle or excipient, and optionally an adjuvant.

The invention also relates to a multivalent immunogenic composition or a multivalent vaccine comprising at least one in vivo expression vector in which at least one polynucleotide of the WN virus is inserted (and expressed in vivo) and at least a second expression vector in which a polynucleotide encoding an immunogen of another pathogenic agent is inserted (and expressed in vivo). Such multivalent compositions or vaccines also comprise or consist essentially of a pharmaceutically or veterinarily acceptable carrier or vehicle or excipient, and optionally an adjuvant.

For antigen(s) or immunogen(s) or epitope(s) to be included in or expressed by a multivalent immunogenic composition or vaccine (in addition to WNV antigen(s), immunogen(s) or epitope(s)), including as to determining or ascertaining epitope(s), the skilled artisan may consult herein cited documents and documents cited in herein cited documents, all of which are incorporated by reference into the instant application.

For equine multivalent immunogenic compositions and multivalent vaccines, the additional equine pathogen(s), as to which additional equine antigen(s) or immunogen(s) or epitope(s) thereof are included in and/or expressed by the multivalent immunogenic compositions and multivalent vaccines, are advantageously chosen from among the group including viruses of equine rhinopneumonia, EHV-1 and/or EHV-4 (and preferably there is a combination of immunogens of EHV-1 and EHV-4), equine influenza virus, EIV, eastern encephalitis virus, EEV, western encephalitis virus, WEV, Venezuelan encephalitis virus, VEV (preference being given to a combination of the three, i.e., EEV, WEV and VEV), Clostridium tetani (tetanus), and mixtures thereof. Preferably, for EHV the immunogen is gB and/or gD see also U.S. Pat. Nos. 6,395,283, 6,248,333, 5,338,683, 6,183,750; for herpesvirus immunogens and constructs expressing the same); for EIV the immunogen is advantageously HA, NP and/or N; for viruses of encephalitis, the immunogen is advantageously C and/or E2; and for Clostridium tetani the immunogen is all or part of the subunit C of the tetanic toxin. Thus, the invention comprehends the use of polynucleotide(s) encoding (an) immunologically active fragment(s) or (an) epitope(s) of such immunogen(s).

For canine multivalent immunogenic compositions and multivalent vaccines, the additional canine pathogen(s), as to which additional canine antigen(s) or immunogen(s) or epitope(s) thereof are included in and/or expressed by the multivalent immunogenic compositions and multivalent vaccines, are advantageously chosen from among the group including viruses of measles disease virus, canine distemper virus (CDV), canine parainfluenza type 2 virus (CPI-2), canine herpesvirus type 1 (CHV-1), rabies virus (rhabdovirus), canine parvovirus (CPV), canine coronavirus (CCV), canine adenovirus, Borrelia burgdorferi, Leptospira and mixtures thereof. Preferably, for CDV the immunogen is advantageously F and/or HA (see also U.S. Pat. Nos. 6,309,647, 5,756,102 regarding CDV immunogens and constructs); for CPV the immunogen is advantageously VP2; for CCV the immunogen is advantageously S and/or M; for CHV-1 the immunogen is advantageously gB and/or gC and/or gD (see also U.S. Pat. Nos. 5,688,920, 5,529,780, regarding CHV immunogens and constructs); for rabies virus the immunogen is advantageously G (see also U.S. Pat. No. 5,843,456 regarding rabies combination compositions); for Borrelia burgdorferi the immunogen is advantageously OspA (see also U.S. Pat. No. 6,368,603 regarding OspA combination compositions). The invention thus comprehends the use of polynucleotide(s) encoding (an) immunologically active fragment(s) or an epitope(s) of such immunogen(s).

For feline multivalent immunogenic compositions and multivalent vaccines, the additional feline pathogen(s), as to which additional feline antigen(s) or immunogen(s) or epitope(s) thereof are included in and/or expressed by the multivalent immunogenic compositions and multivalent vaccines, are advantageously chosen from among the group including viruses of the feline herpesvirus type 1 (FHV-1), feline calicivirus (FCV), rabies virus (rhabdovirus), feline parvovirus (FPV), feline infectious peritonitis virus (FIPV), feline leukaemia virus (FeLV), feline immunodeficiency virus (FIV), Chlamydia and mixtures thereof. Preferably, for FeLV the immunogen is advantageously A and/or B and/or gag and/or pol, e.g., gag/pol; for FPV the immunogen is advantageously VP2; for FIPV the immunogen is advantageously S and/or M and/or N, e.g., S and M and/or N (see also U.S. Pat. Nos. 6,348,196 and 5,858,373 and immunogens and constructs thereof); for FHV the immunogen is advantageously gB and/or gC and/or gD, e.g., gB and gC and/or gD (see also U.S. Pat. Nos. 5,338,683, 6,183,750; for herpesvirus immunogens and constructs expressing the same); for FCV the immunogen is advantageously C; for FIV the immunogen is advantageously env and/or gag and/or pro, e.g., gag/pro, env, or env and gag/pro (see also immunogens and constructs discussed in Tartaglia et al., U.S. application Ser. No. 08/746,668, filed Nov. 14, 1996); for rabies virus the immunogen is advantageously G. The invention thus comprehends the use of polynucleotide(s) encoding (an) immunologically active fragment(s) or (an) epitope(s) of said immunogen(s).

For avian multivalent immunogenic compositions and multivalent vaccines, the additional avian pathogen(s), as to which additional avian antigen(s) or immunogen(s) or epitope(s) thereof are included in and/or expressed by the multivalent immunogenic compositions and multivalent vaccines, are advantageously chosen from among the group including viruses of the Marek's disease virus (MDV) (e.g., serotypes 1 and 2, preferably 1), Newcastle disease virus (NDV), Gumboro disease virus or infectious bursal disease virus (IBDV), infectious bronchitis virus (IBV), infectious anaemia virus or chicken anemia virus (CAV), infectious laryngotracheitis virus (ILTV), encephalomyelitis virus or avian encephalomyelitis virus (AEV or avian leukosis virus ALV), virus of hemorragic enteritis of turkeys (HEV), pneumovirosis virus (TRTV), fowl plague virus (avian influenza), chicken hydropericarditis virus, avian reoviruses, Escherichia coli, Mycoplasma gallinarum, Mycoplasma gallisepticum, Haemophilus avium, Pasteurella gallinarum, Pasteurella multocida gallicida, and mixtures thereof. Preferably, for MDV the immunogen is advantageously gB and/or gD, e.g., gB and gD, for NDV the immunogen is advantageously HN and/or F, e.g., HN and F; for IBDV the immunogen advantageously isVP2; for IBV the immunogen is advantageously S (more advantageously S1) and/or M and/or N, e.g., S (or S1) and M and/or N; for CAV the immunogen is advantageously VP1 and/or VP2; for ILTV the immunogen is advantageously gB and/or gD; for AEV the immunogen advantageously is env and/or gag/pro, e.g., env and gag/pro or gag/pro; for HEV the immunogen is advantageously the 100K protein and/or hexon; for TRTV the immunogen is advantageously F and/or G, and for fowl plague the immunogen is advantageously HA and/or N and/or NP, e.g., HA and N and/or NP. The invention thus comprehends the use of polynucleotide(s) encoding (an) immunologically active fragment(s) or (an) epitope(s) of said immunogen(s).

By way of example, in a multivalent immunogenic composition or a multivalent vaccine according to the invention, to which one or more adjuvants has optionally been added (and hence the composition contains or consists essentially of or consists of one or more adjuvants) as discussed herein, and which is intended for equine species, it is possible to incorporate (and hence for the composition or vaccine to comprise, consist essentially of or consist of) one or more of the plasmids described in WO98/03 198, advantageously as discussed in examples 8 to 25 thereof, and/or those described in WO00/77043 and which relate to the equine species, advantageously those described in examples 6 and 7 thereof. For the canine species, a multivalent composition or vaccine may contain or consist essentially of or consist of one or more of the plasmids described in WO98/03 199, advantageously as discussed in examples 8 to 16 thereof, and/or those described in WO00/77043 and which relate to the canine species, advantageously those described in examples 2, 3 and 4 thereof; and, such compositions or vaccines can contain, consist essentially of or consist of one or more adjuvants. For the feline species, a multivalent composition or vaccine may contain or consist essentially of or consist of one or more of the plasmids described in WO98/03660, advantageously in examples 8 to 19 thereof, and/or those described in WO00/77043 and which relate to the feline species, advantageously those described in example 5 thereof, and, such compositions or vaccines can contain, consist essentially of or consist of one or more adjuvants. And for the avian species, a multivalent composition or vaccine may contain or consist essentially of or consist of one or more of the plasmids described in WO98/03659, advantageously in examples 7 to 27 thereof, and, such compositions or vaccines can contain, consist essentially of or consist of one or more adjuvants.

The immunogenic compositions or vaccines as discussed herein can also be combined with at least one conventional vaccine (e.g., inactivated, live attenuated, or subunit) directed against the same pathogen or at least one other pathogen of the species to which the composition or vaccine is directed. The immunogenic compositions or vaccines discussed herein can be administered prior to or after the conventional vaccine, e.g., in a “prime-boost” regimen.

The invention further comprehends combined vaccination employing immunogenic composition(s) and subunit vaccine(s) according to the invention. Thus, the invention also relates to multivalent immunogenic compositions and multivalent vaccines comprising one or more proteins according to the invention and one or more immunogens (as the term immunogen is discussed herein) of at least one other pathogenic agent (advantageously from among those herein and in documents cited and incorporated herein by reference) and/or another pathogenic agent in inactivated or attenuated form or as a subunit. In the manner described, these multivalent vaccines or compositions also contain, consist essentially of or consist of a pharmaceutically or veterinarily acceptable vehicle or excipient and optionally one or more adjuvants.

The present invention also relates to methods for the immunization and vaccination of a target species, e.g., as discussed herein.

The present invention also relates to methods for the immunization and/or vaccination of a target species, using a prime-boost regimen. The term of “prime-boost” refers to the successive administrations of two different vaccine types or immunogenic or immunological composition types having at least one immunogen in common. The priming administration (priming) is the administration of a first vaccine or immunogenic or immunological composition type and may comprise one, two or more administrations. The boost administration is the administration of a second vaccine or immunogenic or immunological composition type and may comprise one, two or more administrations, and, for instance, may comprise or consist essentially of annual administrations.

An embodiment of a prime-boost immunization or vaccination against WNV according to the invention is a prime-boost immunization or vaccination wherein the animal is first administered a (priming) DNA vaccine or immunological or immunogenic composition comprising or consisting essentially of and expressing in vivo at least one immunogen, antigen or epitope of WNV, and thereafter is administered (boosted with) a second type of vaccine or immunogenic or immunological composition containing or consisting essentially of or expressing at least one immunogen, antigen or epitope that is common to the priming vaccine or immunogenic or immunological composition. This second type of vaccine can be a an inactivated, or attenuated or subunit vaccine or immunogenic or immunological composition or a vector, e.g., recombinant or modified virus vaccine or immunogenic or immunological composition that has in vivo expression (e.g. poxvirus, herpesvirus, adenovirus). Poxviruses may be advantageously employed, e.g., attenuated vaccinia viruses, like MVA or NYVAC, and avipox viruses, like canarypox viruses and fowlpox viruses.

Advantageously, the DNA vaccine is intended to induce a priming immune response specific for the expressed immunogen, antigen or epitope or “DNA induced immune response” (such as a gamma-interferon+(IFN_{γ}+) T cell memory response specific for the expressed immunogen, antigen or epitope) which is boostable (can be boosted) by a subsequent administration (boost) of an inactivated vaccine or immunological composition or a live recombinant vaccine comprising or consisting essentially of a viral vector, such as a live recombinant poxvirus, containing or consisting essentially of and expressing in vivo at least the same immunogen(s) or antigen(s) or epitope(s) expressed by the DNA vaccine. The IFN_{γ}+T cell memory response specific for the expressed WNV immunogen can be shown in a quantitative enzyme-linked immune spot (ELISPOT) assay using peripheral blood mononuclear cells (PBMCs) (Laval F. et al., Vet. Immunol. Iruunopathol., 2002, 90(3-4), 191-201).

The “boost” may be administered from about 2 weeks to about 6 months after the “priming”, such as from about 3 to about 8 weeks after the priming, and advantageously from about 3 to about 6 weeks after the priming, and more advantageously, about 4 weeks after the priming.

For equines, the priming can be done with a DNA vaccine or immunogenic or immunological composition comprising or consisting essentially of and expressing in vivo nucleic acid molecule(s) encoding a WNV immunogen, antigen or epitope according to the invention and the boost is advantageously done with a vaccine or immunogenic or immunological composition comprising a recombinant live viral vector (e.g. poxvirus, herpesvirus, adenovirus), such as a recombinant fowlpox virus or recombinant canarypox virus, recombinant EHV-1 or EHV-4, comprising or consisting essentially of nucleic acid molecule(s) encoding and expressing in vivo at least one of the same WNV immunogen(s), antigen(s) or epitope(s) as the DNA vaccine or immunogenic or immunological composition expresses. In another embodiment these priming and boost vaccines or immunological or immunogenic compositions can be adjuvanted, for instance, by DMRIE-DOPE for the priming DNA vaccine or inmmunological or immunogenic composition and by Carbopol® for the boost recombinant vaccine or immunological or immunogenic composition.

The priming may be performed on a young foal that can have maternal antibodies against WNV (against which immunization or vaccination is directed). Advantageously, the DNA vaccine or immunological or immunogenic composition is administered to the young foal from foaling up to and including about 16 weeks of age, such as from foaling up to and including about 8 weeks of age, for instance, from foaling up to and including about 6 weeks of age, e.g., from foaling up to and including about 4 weeks of age.

For felines, the priming can be done with a DNA vaccine or immunogenic or immunological composition according to the invention comprising or consisting essentially of and expressing in vivo nucleic acid molecule(s) encoding a WNV immunogen, antigen or epitope and the boost is advantageously done with a vaccine or immunogenic or immunological composition comprising or consisting essentially a recombinant live viral vector (e.g. poxvirus, herpesvirus, adenovirus, advantageously recombinant fowlpox virus or recombinant canarypox virus, recombinant FHV, recombinant canine adenovirus), comprising or consisting essentially of nucleic acid molecule(s) encoding and expressing in vivo at least one WNV immunogen, antigen or epitope that is the same as that expressed by the DNA vaccine do. In another embodiment these priming and boost vaccines or immunological or immunogenic compositions can be adjuvanted, for instance, by DMRIE-DOPE for the priming DNA vaccine or immunological or immunogenic composition and by Carbopol® for the boost recombinant vaccine or immunological or immunogenic composition.

The priming may be performed on a young kitten that can have maternal antibodies against WNV (against which immunization or vaccination is directed). The DNA vaccine or immunological or immunogenic composition can be administered to the young kitten from birth up to and including about 12 weeks of age, for instance, from birth up to and including about 8 weeks of age, advantageously from birth up to and including about 6 weeks of age, e.g., from birth up to and including about 4 weeks of age.

For canines, the priming can be done with a DNA vaccine or immunogenic or immunological composition according to the invention comprising or consisting essentially of and expressing in vivo nucleic acid molecule(s) encoding a WNV immunogen, antigen or epitope and the boost is advantageously done with a vaccine or immunogenic or immunological composition comprising or consisting essentially a recombinant live viral vector (e.g. poxvirus, herpesvirus, adenovirus, advantageously recombinant fowlpox virus or recombinant canarypox virus, recombinant CHV, recombinant canine adenovirus), comprising or consisting essentially of nucleic acid molecule(s) encoding and expressing in vivo at least one WNV immunogen, antigen or epitope that is the same as that expressed by the DNA vaccine do. In another embodiment these priming and boost vaccines or immunological or immunogenic compositions can be adjuvanted, for instance, by DMRIE-DOPE for the priming DNA vaccine or immunological or immunogenic composition and by Carbopol® for the boost recombinant vaccine or immunological or immunogenic composition.

The priming may be performed on a young puppy that can have maternal antibodies against WNV (against which immunization or vaccination is directed).The DNA vaccine or immunological or immunogenic composition can be administered to the young puppy from birth up to and including about 12 weeks of age, for instance, from birth up to and including about 8 weeks of age, advantageously from birth up to and including about 6 weeks of age, e.g., from birth up to and including about 4 weeks of age.

For avians, the priming can be done with a DNA vaccine or immunogenic or immunological composition according to the invention comprising or consisting essentially of and expressing in vivo nucleic acid molecule(s) encoding a WNV immunogen, antigen or epitope and the boost is advantageously done with a vaccine or immunogenic or immunological composition comprising or consisting essentially a recombinant live viral vector (e.g. poxvirus, herpesvirus, adenovirus, advantageously recombinant fowlpox virus or recombinant canarypox virus, recombinant HVT, recombinant MDV, recombinant avian adenovirus), comprising or consisting essentially of nucleic acid molecule(s) encoding and expressing in vivo at least one WNV immunogen, antigen or epitope that is the same as that expressed by the DNA vaccine do. In another embodiment these priming and boost vaccines or immunological or immunogenic compositions can be adjuvanted, for instance, by DMRIE-DOPE for the priming DNA vaccine or immunological or immunogenic composition and by Carbopol® for the boost recombinant vaccine or immunological or immunogenic composition.

The priming may be performed on a young avian (bird, e.g., chicken) that can have maternal antibodies against WNV (against which immunization or vaccination is directed).The DNA vaccine or immunological or immunogenic composition can be administered to the young avian (bird, such as chicken) from about one day up to and including about 4 weeks of age, for instance, from one day up to and including about 3 weeks of age; and, the boost is administered from about 2 to about 8 weeks after the priming, advantageously from about 2 weeks to about 4 weeks after priming. For the layers, the boost vaccine or immunological or immunogenic composition may alternatively be administered to about 17 weeks of age for hens, to about 25 weeks of age for ducks and to about 30 weeks of age for turkey hens. Another administration of the boost vaccine or immunological or immunogenic composition can be done before each laying period.

In an embodiment, the priming DNA vaccine or immunological or immunogenic composition comprises or consists essentially of a plasmid encoding and expressing prM-M-E polyprotein, such as the plasmid pFC 115 (example 17), that so encodes and expresses the prM-M-E polyprotein, and the boost recombinant vaccine or immunological or immunogenic composition comprises or consists essentially of a poxvirus such as a canarypox virus, for instance, the recombinant canarypox virus vCP2017 (example 18.1). In another embodiment these priming and boost vaccines or immunological or immunogenic compositions can be adjuvanted: the DNA vaccine or immunological or immunogenic composition containing the plasmid pFC115 can be adjuvanted by DMRIE-DOPE, such as described in example 20; and the recombinant vaccine or immunological or immunogenic composition containing vCP2017 can be adjuvanted by Carbopol®, such as described in example 19.

In a further embodiment, the priming DNA vaccine or immunological or immunogenic composition comprises or consists essentially of a plasmid encoding and expressing prM-M-E polyprotein, such as the plasmid pFC115 (example 17) and the boost recombinant vaccine or immunological or immunogenic composition comprises a poxvirus such as a fowlpox virus, e.g., the recombinant fowlpox virus vFP2000 (example 28). In another embodiment these priming and boost vaccines or immunological or immunogenic compositions can be adjuvanted: the DNA vaccine or immunological or immunogenic composition containing the plasmid pFC115 can be adjuvanted by DMRIE-DOPE, as described in example 20; and the recombinant vaccine or immunological or immunogenic composition containing vFP2000 can be adjuvanted by Carbopol®, as described in example 29.

The invention also relates to kits for performing prime-boost methods comprising or consisting essentially of a priming vaccine or immunological or immunogenic composition and a boost vaccine or immunological or immunogenic compositions in separate containers, optionally with instructions for admixture and/or administration.

The amounts (doses) administered in the priming and the boost and the route of administration for the priming and boost can be as herein discussed, such that from this disclosure and the knowledge in the art, the prime-boost regimen can be practiced without undue experimentation. Furthermore, from the disclosure herein and the knowledge in the art, the skilled artisan can practice the methods, kits, etc. herein with respect to any of the herein-mentioned target species.

These methods can comprise, consist essentially of or consist of the administration of an effective quantity of an immunogenic composition or vaccine according to the invention. This administration can be by the parenteral route, e.g. by subcutaneous, intradermic or intramuscular administration, and/or by oral and/or nasal routes. Advantageously, this administration is intramuscularly or subcutaneously. One or more administrations can take place, such as two administrations.

Vaccines or immunogenic compositions can be injected by a needleless, liquid jet injector or powder jet injector. For plasmids it is also possible to use gold particles coated with plasmid and ejected in such a way as to penetrate the cells of the skin of the subject to be immunized (Tang et al., Nature 1992, 356, 152-154). Other documents cited and incorporated herein may be consulted for administration methods and apparatus of vaccines or immunogenic compositions of the invention. The needleless injector can also be for example Biojector 2000 (Bioject Inc., Portland Oreg., USA).

Advantageously, the immunogenic compositions and vaccines according to the invention comprise or consist essentially of or consist of an effective quantity to elicit an immunological response and/or a protective immunological response of one or more expression vectors and/or polypeptides as discussed herein; and, an effective quantity can be determined from this disclosure, including the documents incorporated herein, and the knowledge in the art, without undue experimentation.

In the case of immunogenic compositions or vaccines based on a plasmid vector, a dose can comprise, consist essentially of or consist of, in general terms, about in 10 μg to about 2000 μg, advantageously about 50 μg to about 1000 μg. The dose volumes can be between about 0.1 and about 2 ml, preferably between about 0.2 and about 1 ml.

These doses and dose volumes are suitable for the vaccination of equines and other target species that are mammals such as canines, felines.

For the vaccination or immunization of an avian, a dose is advantageously between about 10 μg and about 500 μg and preferably between about 50 μg and about 200 μg. The dose volumes can be between about 0.1 and about 1 ml, preferably between about 0.2 and about 0.5 ml.

One skilled in the art can determine the effective plasmid dose to be used for each immunization or vaccination protocol and species from this disclosure and the knowledge in the art.

In the case of immunogenic compositions or vaccines based on a poxvirus, a dose can be between about 10² pfu and about 10⁹ pfu.

For equines and other target species that are mammals such as felines and canines, when the vector is a vaccinia virus, the dose is more advantageously between about 10⁴ pfu and about 10⁹ pfu, preferably between about 10⁶ pfu and about 10⁸ pfu and when the vector is a canarypox virus, the dose is more advantageously between about 10⁵ pfu and about 10⁹ pfu and preferably between about 10^{5.5} pfu or about 10⁶ pfu and about 10⁸ pfu.

For an avian, when the vector is a poxvirus such as a canarypox virus, the dose is more advantageously between about 10³ pfu and about 10⁷ pfu, preferably between about 10⁴ pfu and about 10⁶ pfu; and, when the vector is a poxvirus such as a fowlpox virus, the dose is more advantageously between about 10² pfu and about 10⁵ pfu, preferably between about 10³ pfu and about 10⁵ pfu. From this disclosure and the knowledge in the art, the skilled artisan can determine the suitable dose when the vector is another avipox virus, such as a dovepox, pigeonpox, etc.

In the case of immunogenic compositions or vaccines for a mammalian target species, based on a viral vector other than a poxvirus, such as a herpes viruses or adenovirus, a dose is generally between about 10³ pfu and about 10⁸ pfui; and, in the case of such non-poxvirus-viral-vector-based immunogenic compositions for avian species or avian vaccines, a dose is generally between about 10³ pfu and about 10⁶ pfu. For such non-poxvirus-viral-vector-based immunogenic or vaccine compositions for larger target mammal species, e.g., larger cats (e.g., kept in a zoo) or equines, e.g., in the case of equine immunogenic or vaccine compositions, a dose is advantageously between about 10⁶ pfu and about 10⁸ pfu.

The dose volume of immunogenic and vaccine compositions for target species that are mammals, e.g., the dose volume of equine immunogenic or vaccine compositions, based on viral vectors, e.g., non-poxvirus-viral-vector-based immunogenic or vaccine compositions, is generally between about 0.5 and about 2.5 ml, such as between about 0.5 and about 2.0 ml, preferably between about 1.0 and about 2.0 ml, preferably about 1.0 ml. The dose volume of immunogenic or vaccine compositions for avians based on viral vectors, e.g., the dose volume of non-poxvirus-viral-vector-based avian immunogenic or vaccine compositions, is generally between about 0.1 and about 1.0 ml, preferably between about 0.1 and about 0.5 ml and more advantageously between about 0.2 and about 0.3 ml. Also in connection with such a vaccine or immunogenic composition, from the disclosure herein and the knowledge in the art, the skilled artisan can determine the number of administrations, the administration route, and the doses to be used for each immunization or vaccination protocol, without any undue experimentation. For instance, there can be two administrations to a horse, e.g. at 35 day intervals.

In the case of subunit immunogenic compositions or subunit vaccines, with reference to the amount of active ingredient, e.g., subunit (antigen, immunogen, epitope) a dose comprises or consists essentially of or consists of, in general terms, about 10 μg to about 2000 μg, advantageously about 50 μg to approximately 1000 μg. The dose volume of such immunogenic or vaccine compositions for target species that are mammals, e.g., for equines, is generally between about 1.0 and about 2.0 ml, preferably between about 0.5 and about 2.0 ml and more advantageously about 1.0 ml. The dose volumes of such immunogenic or vaccine compositions avians is generally between about 0.1 and about 1.0 ml, preferably between about 0.1 and about 0.5 ml, and more advantageously between 0.2 and 0.3 ml. Also for such a vaccine or immunogenic composition, the skilled artisan, from this disclosure and the knowledge in the art, can, without any undue experimentation, determine the number of administrations, the administration route and the doses to be used for each immunization or vaccination protocol.

The invention also relates to the use of an in vivo expression vector or a preparation of vectors and/or polypeptides according to the invention, for the formulation of an immunogenic composition or a vaccine intended to protect a target species, or elicit in the target species an immunological response, against the WN virus, and in certain embodiments, against at least one other pathogenic agent.

A vaccine based on plasmid or a viral vaccine expressing one or more proteins of the WN virus or a WN subunit vaccine according to the present invention will not induce in the immunized or vaccinated animal antibodies against other proteins of the virus, which are not presented in or by the immunogenic composition or vaccine (e.g., not present in the immunogenic composition or vaccine and/or not expressed by the immunogenic composition or vaccine). By this feature, the instant invention provides differential diagnostic methods. The present invention makes it possible to make a distinction between animals infected by the WN pathogenic virus and animals vaccinated or immunized with vaccines or compositions according to the invention. In the former, proteins and/or antibodies directed against them are present and can be detected by an antigen-antibody reaction. In the latter (the animals vaccinated or immunized according to the invention), this is not the case, as such animals remain negative in such an antigen-antibody reaction as to proteins not presented in or by the immunogenic or vaccine composition or antibodies thereto. In order to bring about this discrimination, the diagnostic method employs a protein which is not represented in or by the vaccine or immunogenic composition (not present and/or not expressed), e.g. protein C or protein NS1, NS2A, NS2B or NS3 when it is not represented in the vaccine or immunogenic composition.

Accordingly, the instant invention comprehends diagnostic assays or kits that employ a protein or antibody thereto that is not presented in or by a vaccine or immunogenic composition of the invention; and, kits that contain such a diagnostic assay or kit and such a vaccine or immunogenic composition, whereby the user can innoculate and/or vaccinate animals and thereafter test the animals, to determine those animals that have been exposed to WNV vs. those animals that have only been immunuzed and/or vaccinated against WNV.

Thus, the present invention relates to the use of vectors, preparations and polypeptides according to the invention for the preparation of immunogenic compositions and vaccines making it possible to discriminate between vaccinated or immunized animals and infected animals.

The instant invention also relates to an immunization and vaccination method associated with a diagnostic method permitting such a discrimination.

The protein selected for the diagnosis or one of its fragments or epitopes is used as the antigen in the diagnostic test and/or is used for producing polyclonal or monoclonal antibodies.

The one skilled in the art has sufficient practical knowledge to produce these antibodies and to implement antigens and/or antibodies in conventional diagnostic methods, e.g. ELISA tests, and thereby perform differential diagnostic tests according to the instant invention.

The invention will now be further described and illustrated by way of the following, non-limiting examples.

### EXAMPLES

All the constructions are implemented using standard molecular biology methods (cloning, digestion by restriction enzymes, synthesis of a complementary single-strand DNA, polymerase chain reaction, elongation of an oligonucleotide by DNA polymerase, etc.) described by Sambrook J. et al. (Molecular Cloning: A Laboratory Manual, 2nd edition, Cold Spring Harbor Laboratory, Cold Spring Harbor. New York, 1989). All the restriction fragments used for these examples of the present invention, as well as the various polymerase chain reaction (PCR) products are isolated and purified using the Qiagen gel extraction or PCR purification kits

### Example 1

### Culture of the West Nile Fever Virus

For amplification, West Nile fever virus NY99 (Lanciotti R. S. et al., Science, 1999, 286, 2333-7)) are cultured on VERO cells (monkey renal cells), obtainable from the American Type Culture Collection (ATCC) under no. CCL-81.

The VERO cells were cultured in 25 cm² Falcon with eagle-MEM medium supplemented by 1% yeast extracts and 10% calf serum containing approximately 100,000 cells/ml. The cells were cultured at +37° C. under a 5% CO₂ atmosphere.

After three days the cellular layer reaches to confluence. The culture medium was then replaced by the eagle-MEM medium supplemented by 1% yeast extract and 0.1% cattle serum albumin and the West Nile fever virus was added at a rate of 5 pfu/cell.

When the cytopathogenic effect (CPE) was complete (generally 48 to 72 hours after the start of culturing), the viral suspensions were harvested and then clarified by centrifugation and frozen at −70° C. In general, three to four successive passages were necessary for producing a viral batch, which is stored at −70° C.

### Example 1.1

### Construction of an Insertion Plasmid for the Canarypox C5 Locus

FIG. 13 (SEQ ID NO:66) is the sequence of a 5 kb segment of canarypox DNA, encoding an ORF designated C5 initiating at position 1864 and terminating at position 2187. The following describes a C5 insertion plasmid constructed by deleting the majority of the C5 ORF and replacing it with the Virogenetics VQ marker, the H6 promoter, a multiple cloning site (MCS) and transcriptional and translational termination sequences in all reading rames. A 1590 bp PCR fragment, containing the upstream C5R arm is amplified from genomic canarypox DNA using primers C5A1 (SEQ ID NO:67) and C5B1(SEQ ID NO:68). This fragment includes an EcoR I site at the 5′-end, termination sequences and an MCS containing BamH I, Cla I and Xma I sites at the 3′-end. A 458 bp PCR fragment, containing the downstream C5L arm is amplified from genomic canarypox DNA using primers C5C1 (SEQ ID NO:69) and C5D1(SEQ ID NO:70). The fragment includes 5′ BamH I, Cla I and Xma I sites, termination sequences and a Pst I site at the 3′-end. The PCR fragments were fused together by re-amplifying with primers C5A and C5D, generating a 2030 bp EcoR I-Pst I fragment, which is cloned into pUC 8, generating pUC/C5L/B Cla Xm/C5R. Oligonucleotides (SEQ ID NO:71) were used to introduce a unique Not I sequence at the 5′-end of the C5R arm, by inserting into the EcoR I site, generating pUC/Not I/C5R/MCS/C5L.

The Virogenetics VQ marker is contained on plasmid pRW823 and the vaccinia H6 promoter is contained on plasmid pBSH6-1. An 82 bp fragment containing the VQ marker and a 5′ BamH I site, was PCR amplified from pRW823 using primers VQA1 (SEQ ID NO:72) and VQB1 (SEQ ID NO:73). A 176 bp fragment containing the H6 promoter and recognition sequences for a multiple cloning site containing Asp718 I, Xho I, Xba I, Cla I and Sma I, was amplified using primers H6A1 (SEQ ID NO:74) and H6B1 (SEQ ID NO:75). The VQ and H6 fragments were pooled and re-amplified using primers VQA1 and H6B1 to generate a 232 bp VQ/H6p/MCS fragment (FIG. 14, SEQ ID NO:76) that was inserted into pUC/C5L/B Cla Xm/C5R between the BamH I and Xma I sites. FIG. 15 shows the resultant plasmid, pNVQH6C5LSP-18, a C5 insertion plasmid containing the H6 promoter, transcription and translation terminators functional in all reading frames, and a MCS.

Sequences of the PCR primers and oligonucleotides:

| | |
|---|---|
| Primer C5A1 (SEQ ID NO:67) | 5′ GGCCGAATTCTGAATGTTAAATGTTATACTTT 3′ |
| Primer C5B1 (SEQ ID NO:68) | 5′ CCCGGGATCGATGGATCCTTTTTATAGCTAATTAGTCACGTACCTTTGAGAGTACCACT |
| | TCAGCTA 3′ |
| Primer C5C1 (SEQ ID NO:69) | 5′ GGATCCATCGATCCCGGGTTTTTATGACTAGTTAATCACGGCCGCTTATAAAGATCTAA |
| | AATGCAT 3′ |
| Primer C5D1 (SEQ ID NO:70) | 5′ GGCTGCAGGTATTCTAAACTAGGAATAGAT 3′ |
| Oligonucleotide for Not I (SEQ ID NO:71) | 5′ AATTGCGGCCGC 3′ |
| Primer VQA1 (SEQ ID NO:72) | 5′ AAAGGATCCGGGTTAATTAATTAGTCATC 3′ |
| Primer VQB1 (SEQ ID NO:73) | 5′ AATAAAGAAGCTCTAATTAATTAACGAGCAGATA 3′ |
| Primer H6A1 (SEQ ID NO:74) | 5′ TCGTTAATTAATTAGAGCTTCTTTATTCTATACTTAAAAAG 3′ |
| Primer H6B1 (SEQ ID NO:75) | 5′ AAAACCCGGGATCGATTCTAGACTCGAGGGTACCTACGATACAAACTTAACGGATA 3′ |

### Example 2

### Extraction of Viral RNA from the West Nile Fever Virus

The viral RNA contained in 100 ml of viral suspension of the West Nile fever virus strain NY99 was extracted after thawing with solutions of the High Pure Viral RNA Kit Cat # 1 858 882, Roche Molecular Biochemicals, whilst following the instructions of the supplier for the extraction stages. The RNA sediment obtained at the end of extraction was resuspended with 1 to 2 ml of RNase-free, sterile distilled water.

### Example 3

### Construction of Plasmid pFC101

The complementary DNA (cDNA) of the West Nile fever virus NY99 was synthesized with the Gene Amp RNA PCR Kit (Cat # N 808 0017, Perkin-Elmer, Norwalk, Conn. 06859, USA) using the conditions supplied by the manufacture.

A reverse transcriptase polymerase chain reaction (RT-PCR reaction) was carried out with 50 μl of viral RNA suspension of the West Nile fever virus NY99 (Example 2) and with the following oligonucleotides:

| | |
|---|---|
| FC101 (30 mer) (SEQ ID NO:1) | 5′TTTTTTGAATTCGTTACCCTCTCTAACTTC3′ |
| and FC102 (33 mer) (SEQ ID NO:2) | 5′TTTTTTTCTAGATTACCTCCGACTGCGTCTTGA3′ |

This pair of oligonucleotides allows the incorporation of an EcoRI restriction site, a XbaI restriction site and a stop codon at 3′ of the insert.

The synthesis of the first cDNA strand takes place by elongation of oligonucleotide FC102, following the hybridization of the latter with the RNA matrix.

The synthesis conditions of the first cDNA strand were a temperature of 42° C. for 15 min, then 99° C. for 5 min and finally 4° C. for 5 min. The conditions of the PCR reaction in the presence of the pair of oligonucleotides FC101 and FC102 were a temperature of 95° C. for 2 min, then 35 cycles (95° C. for 1 min, then 62° C. for 1 min and 72° C. for 2 min) and finally 72° C. for 7 min to produce a 302 bp fragment.

This fragment was digested by EcoRI and then by XbaI in order to isolate, following agarose gel electrophoresis, the approximately 290 bp EcoRI-XbaI fragment, which was called fragment A.

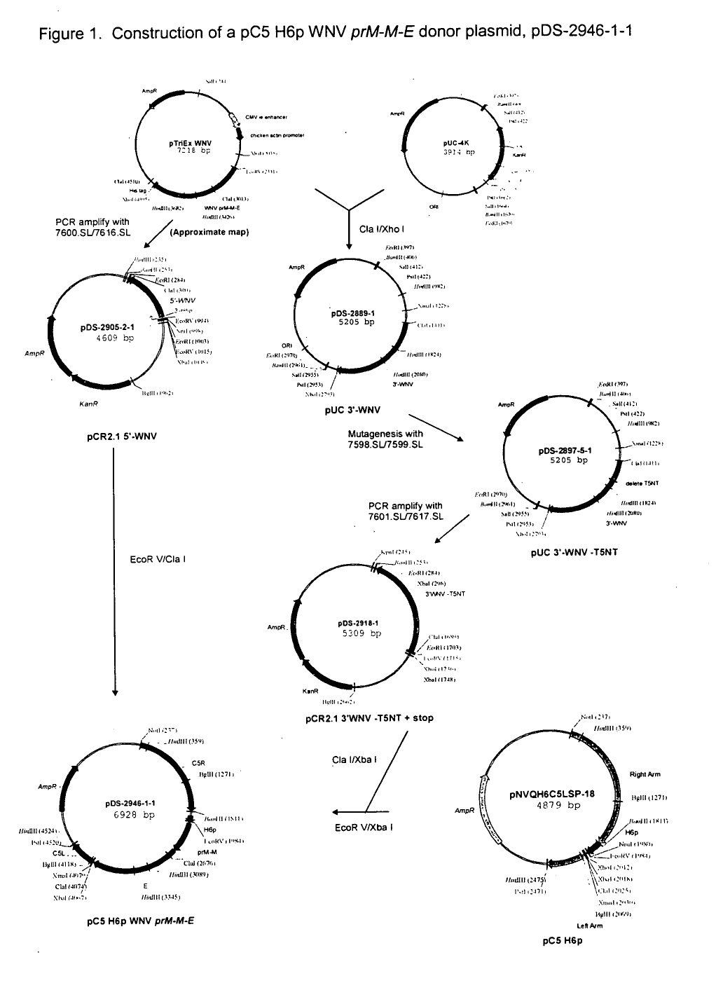

The pVR1020 eukaryotic expression plasmid (C. J. Luke et al. of Infectious Diseases, 1997, 175, 95-97) derived from the plasmid pVR1012 (FIG. 1 and example 7 of WO98/03199—Hartikka J. et al., 1997, Human Gene Therapy, 7, 1205-1217), contains the frame encoding the signal sequence of the human tissue plasminogen activator (tPA).

A pVR1020 plasmid was modified by BamHI-BgIII digestion and insertion of a sequence containing several cloning sites (BamHI, NotI, EcoRI, XbaI, PmlI, PstI, BglII) resulting from hybridization of the following oligonucleotides.

| | |
|---|---|
| BP326 (40 mer) (SEQ ID NO: 3) | 5′GATCTGCAGCACGTGTCTAGAGGATATCGAATTCGCGGCC3′ and |
| BP329 (40 mer) (SEQ ID NO: 4) | 5′GATCCGCGGCCGCGAATTCGATATCCTCTAGACACGTGCT3′ |

The thus obtained vector with a size of approximately 5105 base pairs (or bp) was called pAB110.

Fragment A was ligated with the pAB110 expression plasmid previously digested by XbaI and EcoRI, in order to give the plasmid pFC101 (5376 bp). Under the control of the early promoter of human cytomegalovirus or hCMV-IE (human Cytomegalovirus Immediate Early), the plasmid contains an insert encoding the signal sequence of the activator of tPA followed by the sequence encoding the protein prM.

### Example 3.1

### Construction of a DNA Immunization Vector, pVR1012 WNV prM-M-E, pSL-5448-1-1.

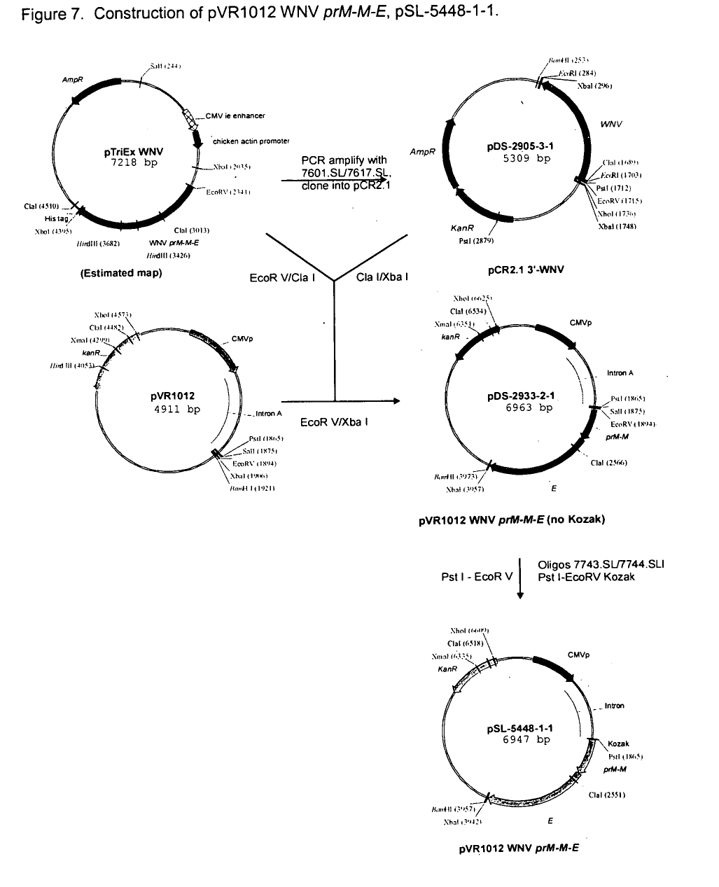

The construction scheme is shown in FIG. 7.

Plasmid pTriEx-WNV containing the NY99 WNV prM-M-E genes, was received from Cornell University. There is a poly-His tag at the 3′ end of the E gene. A 1.2 kb Cla I-Xba I 3′-WNV fragment was PCR amplified using primers 7601.SL and 7617.SL, to remove the poly-His tag and introduce a stop codon and Xba I site for cloning. The resultant fragment was cloned into pCR 2.1, generating pDS-2905-3-1.

Plasmid pVR1012 is a DNA immunization vector containing the human CMV promoter, introna, a multiple cloning site, and a kanamycin resistance gene and has been described by Hartikka et al (Human Gene Therapy 7:1205-1217, 1997). The pVR1012 vector was digested with EcoR V and Xba I and ligated with the 1.2 kb Cla I-Xba I 3′-insert from pDS-2905-3-1 and a 0.7 kb EcoR V-Cla I 5′-fragment from pTri-Ex-WNV, to generate pDS-2933-2-2.

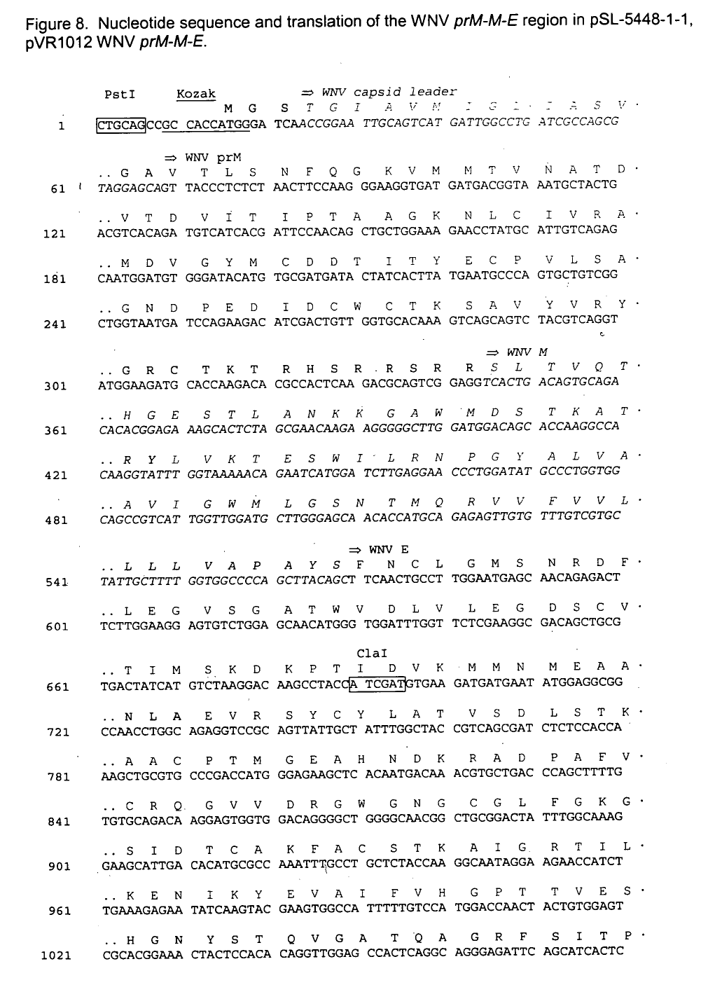

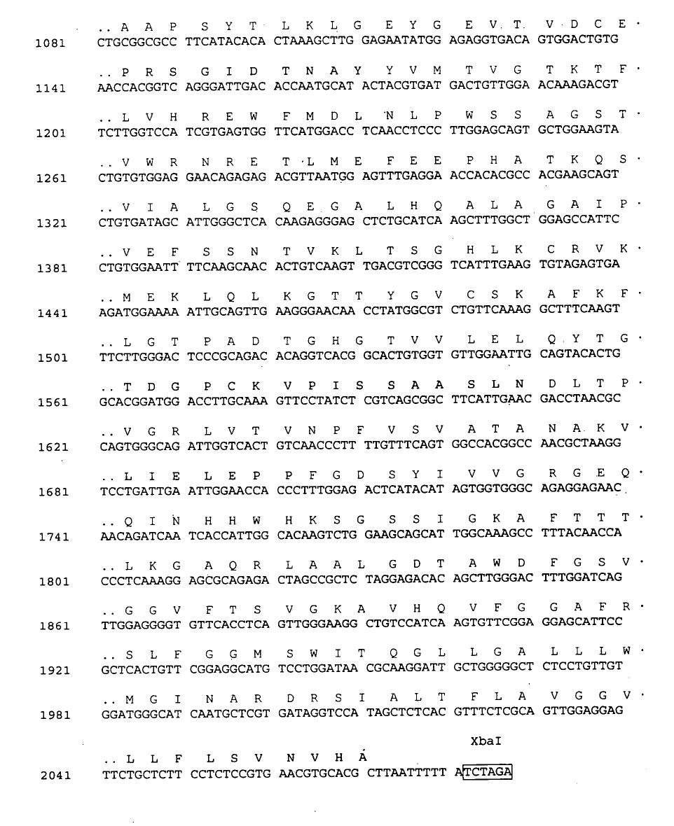

In order to introduce a 5′ Kozak sequence, the Pst I-EcoR V fragment of pDS-2933-2-2 was replaced by annealed oligonucleotides 7743.SL and 7744.SL, generating clone pSL-5448-1-1, pVR1012 prM-M-E. The sequence of the WNVprM-M-E region is shown in FIG. 8.

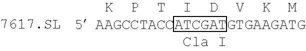

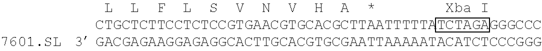

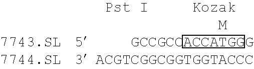

### Example 3.2

### Analysis of the Immunogenicity of pSL-5448-1-1, pVR1012 WNV prM-M-E in Mice

Six to eight week old BALB/c mice were immunized intramuscularly with 100 μg of plasmid DNA in PBS, on days 0, 14, 28, and 42. Bleeds were taken on days 0, 28, 42 and 56, processed and analysed by immunoblot.

Samples for immunoblot analysis were prepared by transient transfection of Chinese Hamster Ovary (CHO) cells with pTriEx-WNV DNA. CHO cells were transfected with 10 μg of pTriEx-WNV DNA using electroporation at settings 1.5 kV, 25 uF and infinite resistance. Mock samples were prepared by the electroporation of CHO cells at the same settings, without DNA. After approximately 65 hours, supernatants were harvested and clarified by spinning at 3000 rpm for 5 min. The plates were washed twice with PBS, then 500 μl of PBS was added and the cells were scraped off. After spinning at 3000 rpm for 5 min, the supernatant was removed and the cells resuspended in 100 μl of SDS-PAGE lysis buffer.

Immunoblots were performed using the CHO/pTriEx-WNV and CHO/mock pellets to assess antigenicity and specificity of the mouse antisera. Samples were suspended in SDS-PAGE loading buffer minus β-mercaptoethanol and separated on a 12% SDS-PAGE gel before electrotransfer to immobilon P nylon membrane. The membranes were processed and probed with 1:1000 dilution of mouse anti-WNV antisera. Peroxidase-conjugated goat anti-mouse antisera was used as secondary antibody and bands were visualized using luminol reagents (NEN). All five mouse anti-WNV antisera reacted specifically with a single protein band in the pTriEx-WNV samples, of the expected size for the WNV E protein. None of the five antisera reacted with anything in the mock samples.

The serum samples were also assayed for virus neutralizing antibodies and titres were found to range between 1:8 and 1:128.

### Example 4

### Construction of Plasmid pFC102

The complementary DNA (cDNA) of the West Nile fever virus NY99 was synthesized with the Gene Amp RNA PCR Kit (Cat # N 808 0017, Perkin-Elmer, Norwalk, Conn. 06859, USA) using the conditions provided by the supplier.

A reverse transcriptase polymerase chain reaction (RT-PCR reaction) was carried out with 50 μl of viral RNA suspension of the West Nile fever virus NY99 (Example 2) and with the following oligonucleotides:

| | |
|---|---|
| FC103 (30 mer) (SEQ ID NO: 5) | 5′TTTTTTGAATTCTCACTGACAGTGCAGACA3′ |
| and FC104 (33 mer) (SEQ ID NO: 6) | 5′TTTTTTTCTAGATTAGCTGTAAGCTGGGGCCAC3′ |

This pair of oligonucleotides allows the incorporation of an EcoRi restriction site and a XbaI restriction site and a stop codon at 3′ of the insert.

The first CDNA strand was synthesized by elongation of oligonucleotide FC104, following the hybridization of the latter on the RNA matrix.

The synthesis conditions of the first cDNA strand were a temperature of 42° C. for 15 min, then 99° C. for 5 min and finally 4° C for 5 min. The conditions of the PCR reaction in the presence of the pair of oligonucleotides FC103 and FC104 were a temperature of 95° C. for 2 min, then 35 cycles (95° C. for 1 min, then 62° C. for 1 min and 72° C. for 2 min) and finally 72° C. for 7 min to produce a 252 bp fragment.

This fragment was digested by EcoRI and then XbaI in order to isolate, following agarose gel electrophoresis, the approximately 240 bp EcoRI-XbaI fragment. This fragment was ligated with the pAB110 expression plasmid (Example 3) previously digested by XbaI and EcoRI in order to give the plasmid pFC102 (5326 bp). Under the control of the early human cytomegalovirus or hCMV-IE (human Cytomegalovirus Immediate Early) promoter, this plasmid contains an insert encoding the signal sequence of the activator of tPA, followed by the sequence encoding the protein M.

### Example 5

### Construction of Plasmid pFC103

The complementary DNA (cDNA) of the West Nile fever virus NY99 was synthesized with the Gene Amp RNA PCR Kit (Cat # N 808 0017, Perkin-Elmer, Norwalk, Conn. 06859, USA) using the conditions provided by the supplier.

A reverse transcriptase polymerase chain reaction (RT-PCR reaction) was carried out with 50 μl of viral RNA suspension of the West Nile fever virus NY99 (Example 2) and with the following oligonucleotides:

| | |
|---|---|
| FC105 (30 mer) (SEQ ID NO: 7) | 5′TTTTTTGAATTCTTCAACTGCCTTGGAATG3′ |
| and FC106 (33 mer) (SEQ ID NO: 8) | 5′TTTTTTTCTAGATTAAGCGTGCACGTTCACGGA3′. |

This pair of oligonucleotides allows the incorporation of an EcoRi restriction site and a XbaI restriction site, together with a stop codon at 3′ of the insert.

The synthesis of the first cDNA strand takes place by elongation of oligonucleotide FC106, following its hybridization with the RNA matrix.

The synthesis conditions of the first cDNA strand were a temperature of 42° C. for 15 min, then 99° C. for 5 min and finally 4° C. for 5 min. The PCR reaction conditions in the presence of the pair of oligonucleotides FC105 and FC106 were a temperature of 95° C. for 2 min, then 35 cycles (95° C. for 1 min, then 62° C. for 1 min and 72° C. for 2 min), and finally 72° C. 7 min for producing a 1530 bp fragment.

This fragment was digested by EcoRI and then by XbaI in order to isolate, following agarose gel electrophoresis, the approximately 1518 bp EcoRI-XbaI fragment. This fragment was ligated with the pAB 110 expression plasmid (Example 3) previously digested by XbaI and EcoRI in order to give the plasmid pFC103 (6604 bp). Under the control of the early promoter of human cytomegalovirus or hCMV-IE (human Cytomegalovirus Immediate Early), the plasmid contains an insert encoding the signal sequence of the activator of tPA, followed by the sequence encoding the protein E.

### Example 6

### Construction of Plasmid pFC104

The complementary DNA (cDNA) of the West Nile fever virus NY99 was synthesized with the Gene Amp RNA PCR Kit (Cat # N 808 0017, Perkin-Elmer, Norwalk, Conn. 06859, USA) using the conditions provided by the supplier.

A reverse transcriptase polymerase chain reaction (RT-PCR reaction) was carried out with 50 μl of viral RNA suspension of the West Nile fever virus NY99 (Example 2) and with the following oligonucleotides:

FC101 (30 mer) (SEQ ID NO: 1)

and FC106 (33 mer) (SEQ ID NO: 8)

This pair of oligonucleotides allows the incorporation of an EcoRI restriction site, a XbaI restriction site and a stop codon at 3′ of the insert.

Synthesis of the first cDNA strand takes place by elongation of oligonucleotide FC106, following its hybridization with the RNA matrix.

The synthesis conditions of the first cDNA strand were a temperature of 42° C. for 15 min, then 99° C. for 5 min and finally 4° C. for 5 min. The PCR reaction conditions in the presence of the pair of oligonucleotides FC101 and FC106 are a temperature of 95° C. for 2 min, then 35 cycles (95° C. for 1 min, then 62° C. for 1 min and 72° C. for 2 min) and finally 72° C. for 7 min in order to produce a 2031 bp fragment.

This fragment was digested by EcoRI and then XbaI in order to isolate, following agarose gel electrophoresis, the approximately 2019 bp EcoRI-XbaI fragment. This fragment was ligated with the pAB110 expression plasmid (Example 3), previously digested by XbaI and EcoRI in order to give the pFC 104 plasmid (7105 bp). Under the control of the early human cytomegalovirus promoter or hCMV-IE (human Cytomegalovirus rmmediate Early), the plasmid contains an insert encoding the signal sequence of the activator of tPA, followed by the sequence encoding the protein prM-M-E.

### Example 7

### Construction of Plasmid pFC105

The complementary DNA (cDNA) of the West Nile fever virus NY99 was synthesized with the Gene Amp RNA PCR Kit (Cat # N 808 0017, Perkin-Elmer, Norwalk, Conn. 06859, USA) using the conditions provided by the supplier.

A reverse transcriptase polymerase chain reaction (RT-PCR reaction) was carried out with 50 μl of viral RNA suspension of the West Nile fever virus NY99 (Example 2) and with the following oligonucleotides:

| | |
|---|---|
| FC107 (36 mer) (SEQ ID NO: 9) | 5′TTTTTTGATATCACCGGAATTGCAGTCATGATTGGC3′ |
| and FC106 (33 mer) (SEQ ID NO: 8). | |

This pair of oligonucleotides allows the incorporation of an EcoRV restriction site, a XbaI restriction site and a stop codon at 3′ of the insert.

Synthesis of the first cDNA strand takes place by elongation of the FC106 oligonucleotide, following its hybridization with the RNA matrix.

The synthesis conditions of the first cDNA strand were a temperature of 42° C. for 15 min, then 99° C. for 5 min and finally 4° C. for 5 min. The PCR reaction conditions in the presence of the pair of oligonucleotides FC106 and FC107 are a temperature of 95° C. for 2 min, then 35 cycles (95° C. for 1 min, then 62° C. for 1 min and 72° C for 2 min) and finally 72° C. for 7 min in order to produce a 2076 bp fragment.

This fragment was digested by EcoRV and then XbaI in order to isolate, following agarose gel electrophoresis, the approximately 2058 bp EcoRV-XbaI fragment.

This fragment was ligated with the pVR1012 expression plasmid, previously digested by XbaI and EcoRV, in order to give the plasmid pFC105 (6953 bp). Under the control of the early human cytomegalovirus promoter or hCMV-IE (human Cytomegalovirus Immediate Early), this plasmid contains an insert encoding the polyprotein prM-M-E.

### Example 8

### Construction of Plasmid pFC106

The complementary DNA (CDNA) of the West Nile fever virus NY99 was synthesized with the Gene Amp RNA PCR Kit (Cat # N 808 0017, Perkin-Elmer, Norwalk, Conn. 06859, USA) using the conditions provided by the supplier.

A reverse transcriptase polymerase chain reaction (RT-PCR reaction) was carried out with 50 μl of viral RNA suspension of the West Nile fever virus NY99 (example 2) and with the following oligonucleotides:

| | |
|---|---|
| FC108 (36 mer) | (SEQ ID NO: 10) |
| 5′TTTTTTGATATCATGTATAATGCTGATATGATTGAC3′ | |
| and | |
| FC109 (36 mer) | (SEQ ID NO: 11) |
| 5′TTTTTTTCTAGATTAACGTTTTCCCGAGGCGAAGTC3′ | |

This pair of oligonucleotides allows the incorporation of an EcoRV restriction site, a XbaI restriction site, an initiating ATG codon in 5′ and a stop codon at 3′ of the insert.

Synthesis of the first cDNA strand takes place by elongation of the oligonucleotide FC109, following its hybridization with the RNA matrix.

The synthesis conditions of the first cDNA strand were a temperature of 42° C. for 15 min, then 99° C. for 5 min and finally 4° C. for 5 min. The PCR reaction conditions in the presence of the pair of nucleotides FC108 and FC109 are a temperature of 95° C. for 2 min, then 35 cycles (95° C. for 1 min, 62° C. for 1 min and then 72° C. for 2 min) and finally 72° C. for 7 min to produce a 2973 bp fragment.

This fragment was digested by EcoRV and then XbaI in order to isolate, following agarose gel electrophoresis, the approximately 2955 bp EcoRV-XbaI fragment.

This fragment was ligated with the pVR 1012 expression plasmid previously digested by XbaI and EcoRV in order to give the plasmid pFC106 (7850 bp). Under the control of the early human cytomegalovirus promoter or hCMV-IE (human Cytomegalovirus Immediate Early), this plasmid contains an insert encoding the polyprotein NS2A-NS2B-NS3.

### Example 9

### Construction of Donor Plasmid for Insertion into C5 site of Canarypox Virus (ALVAC)

FIG. 16 of U.S. Pat. No. 5,756,103 shows the sequence of a genomic DNA 3199 bp fragment of the canarypox virus. Analysis of this sequence has revealed an open reading frame (ORF) called C5.H, which starts at position 1538 and ends at position 1859. The construction of an insertion plasmid leading to the deletion of the ORF C5.H and its replacement by a multiple cloning site flanked by transcription and translation stop signals was implemented in the following way.

A PCR reaction was performed on the basis of the matrix constituted by genomic DNA of the canarypox virus and with the following oligonucleotides:

| | |
|---|---|
| C5A1 (42 mer) (SEQ ID NO: 12): | 5′ATCATCGAGCTCCAGCTGTAATTCATGGTCGAAAAGAAGTGC3′ |
| and C5B1 (73 mer) (SEQ ID NO: 13): | 5′GAATTCCTCGAGCTGCAGCCCGGGTTTTTATAGCTAATTAGTCATTTTTTGAGAGTA |
| | CCACTTCAGCTACCTC3′ |

in order to isolate a 223 bp PCR fragment (fragment B).

A PCR reaction was carried out on the basis of the matrix constituted by genomic DNA of the canarypox virus and with the following oligonucleotides:

| | |
|---|---|
| C5C1 (72 mer) (SEQ ID NO: 14): | 5′CCCGGGCTGCAGCTCGAGGAATTCTTTTTATTGATTAACTAGTCATTATAAAGATC |
| | TAAAATGCATAATTTC3′ |
| and C5D1 (45 mer) (SEQ ID NO: 15): | 5′GATGATGGTACCGTAAACAAATATAATGAAAAGTATTCTAAACTA3′ |

in order to isolate a 482 bp PCR fragment (fragment C).

Fragments B and C were hybridized together in order to serve as a matrix for a PCR reaction performed with the oligonucleotides C5A1 (SEQ ID NO: 12) and C5D1 (SEQ ID NO: 15) in order to generate a 681 bp PCR fragment. This fragment was digested by the restriction enzymes SacI and KpnI in order to isolate, following agarose gel electrophoresis, a 664 bp SacI-KpnI fragment. This fragment was ligated with the pBlueScript® II SK+vector (Stratagene, La Jolla, USA, Cat # 212205), previously digested by the restriction enzymes Sacd and KpnI, in order to give the plasmid pC5.H. The sequence of this plasmid was verified by sequencing. This plasmid contains 166 bp of sequences upstream of ORF C5.H (left flanking arm C5L.H), an early transcription stop signal, stop codons in 6 reading frames, a multiple cloning site containing restriction sites Smal, PstI, XhoI and EcoRI and finally 425 bp of sequences located downstream of ORF C5.H (right flanking arm C5R.H).

The plasmid pMP528HRH (Perkus M. et al. J. Virol. 1989, 63, 3829-3836) was used as the matrix for amplifying the complete sequence of the vaccinia promoter H6 (GenBank access no. M28351) with the following oligonucleotides:

| | |
|---|---|
| JCA291 (34 mer) (SEQ ID NO: 16) | 5′AAACCCGGGTTCTTTATTCTATACTTAAAAAGTG3′ |
| and JCA292 (43 mer) (SEQ ID NO: 17) | 5′AAAAGAATTCGTCGACTACGATACAAACTTAACGGATATCGCG3′ |

in order to amplify a 149 bp PCR fragment. This fragment was digested by restriction enzymes SmaI and EcoRI in order to isolate, following agarose gel electrophoresis, a 138 bp Smal-EcoRI restriction fragment. This fragment was then ligated with the plasmid pC5, previously digested by Smal and EcoRI, in order to give the plasmid pFC 107.

### Example 10

### Construction of the Recombinant Virus vCP1712

A PCR reaction was performed using the plasmid pFC105 (example 7) as the matrix and the following oligonucleotides:

| | |
|---|---|
| FC110 (33 mer (SEQ ID NO: 18): | 5′TTTTCGCGAACCGGAATTGCAGTCATGATTGGC3′ |
| and FC111 (39 mer) (SEQ ID NO: 19): | 5′TTTTGTCGACGCGGCCGCTTAAGCGTGCACGTTCACGGA3′ |

in order to amplify an approximately 2079 bp PCR fragment. This fragment was digested by restriction enzymes NruI and SalI in order to isolate, following agarose gel electrophoresis, an approximately 2068 bp NruI-SalI restriction fragment. This fragment was then ligated with plasmid pFC107 (example 9) previously digested by restriction enzymes NruI and SalI in order to give the plasmid pFC108, which contains C5L-H6p-WNV prM-M-E-C5R.

Plasmid pFC108 was linearized by NotI, then transfected in primary chicken embryo cells. The cells were then infected with the canarypox virus (ALVAC strain) according to the previously described calcium phosphate precipitation method (Panicali and Paoletti, Proc. Nat. Acad. Sci. 1982, 79,4927-4931; Piccini et al. In Methods in Enzymology, 1987, 153, 545-563, publishers Wu R. and Grossman L. Academic Press). Positive plaques were selected on the basis of a hybridization with a radioactively labelled probe specific to the nucleotide sequence of the envelope glycoprotein E. These plaques underwent 2-4 successive selection/purification cycles until a pure population was isolated. A representative plaque corresponding to in vitro recombination between the donor plasmid pFC108 and the genome of the ALVAC canarypox virus was then amplified and the recombinant virus stock obtained was designated vCP1712. (The actual vCP2017, which contains the full-length promoter and signal sequence, was derived after two rounds of screening).

### Example 11

### Construction of the Recombinant Virus vCP1713

Plasmid pFC104 (Example 6) was digested by the restriction enzyme SalI and PmlI in order to isolate, following agarose gel electrophoresis, an approximately 2213 bp PmlI-SalI restriction fragment. This fragment was ligated with plasmid pFC107 (Example 9) previously digested by the NruI and Sall restriction enzymes in order to give the plasmid pFC109.

Plasmid pFC109 was linearized by NotI, then transfected in primary chicken embryo cells infected with the canarypox virus (ALVAC strain) according to the method of Example 10. A representative plaque corresponding to in vitro recombination between the donor plasmid pFC109 and the genome of the ALVAC canarypox virus was selected on the basis of a hybridization of a radioactively labelled probe specific to the nucleotide sequence of the envelope glycoprotein E and was then amplified. The recombinant virus stock obtained was designated vCP1713, ALVAC WNV prM-M-E.

### Example 12

### Construction of the Recombinant Virus vCP1714

Plasmid pFC103 (Example 5) was digested by the Sall and PmlI restriction enzymes in order to isolate, following agarose gel electrophoresis, an approximately 1712 bp PmlI-SalI restriction fragment. This fragment was ligated with the plasmid pFC107 (Example 9) previously digested by the NruI and Sall restriction enzymes in order to give the plasmid pFC110.

Plasmid pFC110 was linearized by NotI, then transfected in primary chicken embryo cells infected with the canarypox virus (ALVAC strain) according to the method of example 10. A representative plaque corresponding to in vitro recombination between the donor plasmid pFC110 and the genome of the ALVAC canarypox virus was selected on the basis of a hybridization with a radioactively labelled probe specific to the nucleotide sequence of the envelope glycoprotein E and was then amplified. The recombinant virus stock obtained was then designated vCP1714, ALVAC WNV E.

### Example 13

### Construction of the Recombinant Virus vCP1715

Plasmid pFC102 (Example 4) was digested by the Sall and PmlI restriction enzymes in order to isolate, following agarose gel electrophoresis, an approximately 434 bp PmIl-SalI restriction fragment. This fragment was ligated with the plasmid pFC107 (Example 9) previously digested by the NruI and Sall restriction enzymes to give the plasmid pFC111.

Plasmid pFC111 was linearized by NotI, then transfected in primary chicken embryo cells infected with the canarypox virus (ALVAC strain) according to the method of Example 10. A representative plaque corresponding to in vitro recombination between the donor plasmid pFC111 and the genome of the ALVAC canarypox virus was selected on the basis of hybridization with a radioactively labelled probe specific to the nucleotide sequence of the membrane M glycoprotein and was then amplified. The recombinant virus stock obtained was designated vCP1715, ALVAC WNV M.

### Example 14

### Construction of the Recombinant Virus vCP1716

Plasmid pFC101 (Example 3) is digested by the SalI and PmlI restriction enzymes in order to isolate, following agarose gel electrophoresis, an approximately 484 bp PmlI-SalI restriction fragment. This fragment is ligated with the plasmid pFC107 (Example 9) previously digested by the NruI and Sall restriction enzymes to give the plasmid pFC112.

Plasmid pFC112 was linearized by NotI and then transfected in primary chicken embryo cells infected with the canarypox virus (ALVAC strain) according to the method of Example 10. A representative plaque corresponding to in vitro recombination between the donor plasmid pFC112 and the genome of the ALVAC canarypox virus was selected on the basis of a hybridization with a radioactively labelled probe specific to the nucleotide sequence of the pre-membrane prM glycoprotein and was then amplified. The recombinant virus stock obtained was designated vCP1716, ALVAC WNV prM.

### Example 15

### Construction of Donor Plasmid for Insertion into C6 Site of Canarypox Virus (ALVAC)

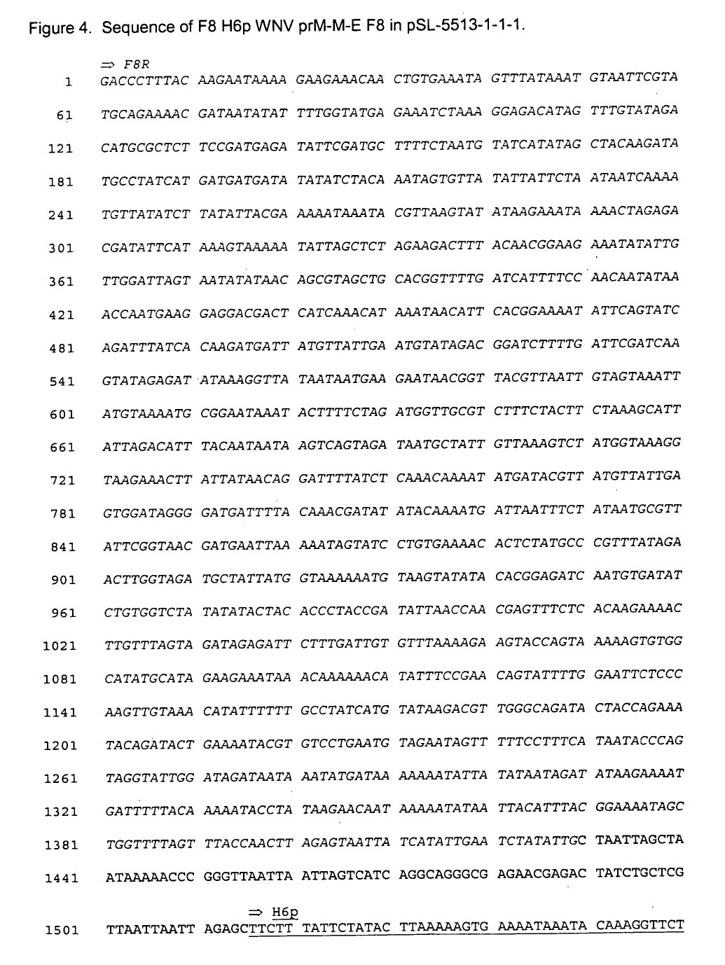

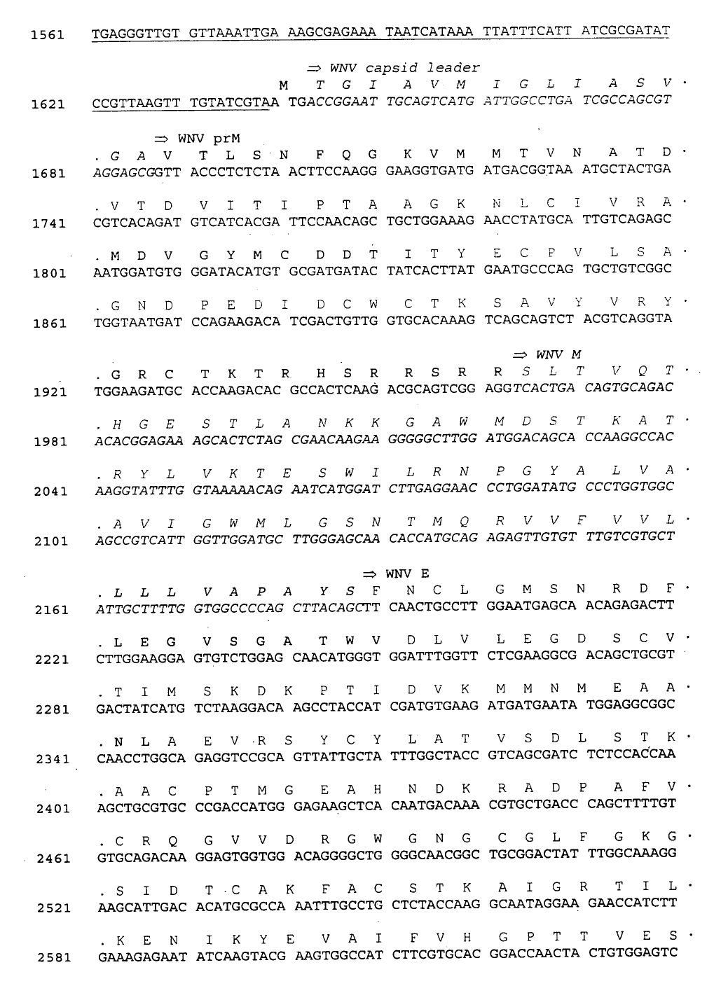

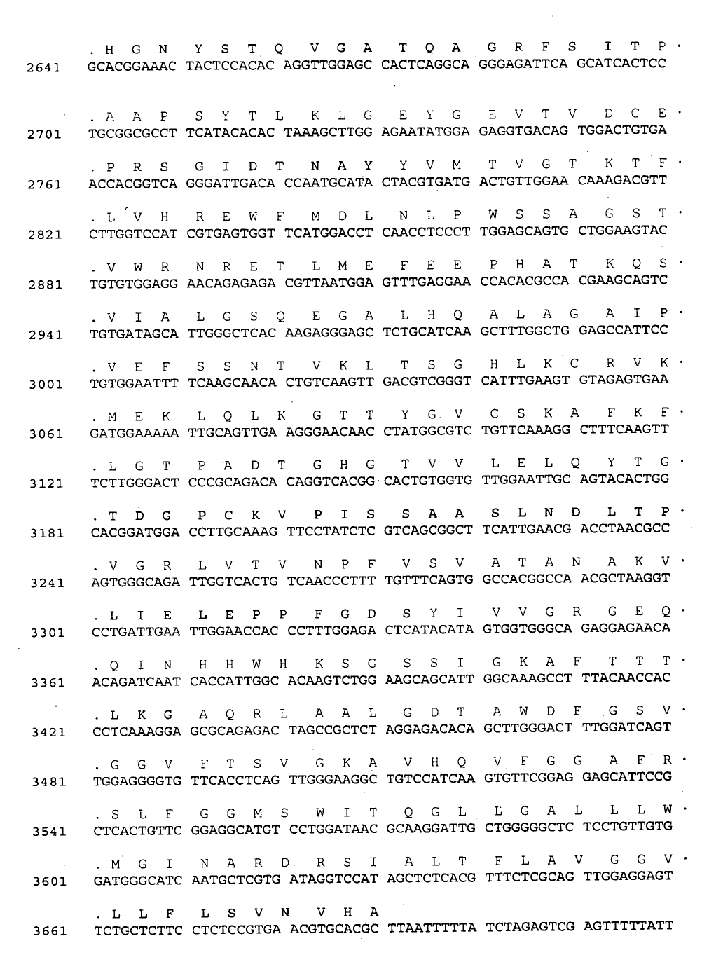

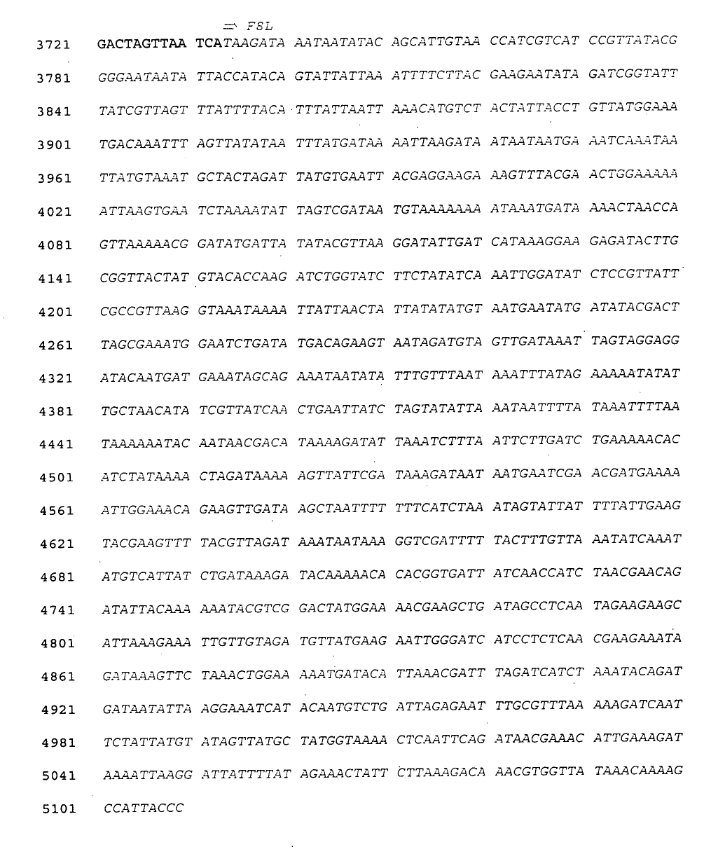

FIG. 4 of WO01/05934 (see also Audonnet et al., allowed U.S. application Ser. No. 09/617,594, filed Jul. 14, 2000, now U.S. Pat. No. 6,541,458 issued Apr. 1, 2003) shows the sequence of a 3700 bp genomic DNA fragment of the canarypox virus. Analysis of this sequence revealed an open reading frame (ORF) called C6, which starts at position 377 and ends at position 2254. The construction of an insertion plasmid leading to the deletion of the ORF C6 and its replacement by a multiple cloning site flanked by transcription and translation stop signals was implemented in the following way.

A PCR reaction was performed on the basis of the matrix constituted by the genomic DNA of the canarypox virus and with the following oligonucleotides:

| | |
|---|---|
| C6A1 (42 mer) (SEQ ID NO: 20): | 5′ATCATCGAGCTCGCGGCCGCCTATCAAAAGTCTTAATGAGTT3′ |
| and C6B1 (73 mer) (SEQ ID NO: 21): | 5′GAATTCCTCGAGCTGCAGCCCGGGTTTTTATAGCTAATTAGTCATTTTTTCGTAAGT |
| | AAGTATTTTTATTTAA3′ |

to isolate a 432 bp PCR fragment (fragment D).

A PCR reaction was performed on the basis of the matrix constituted by the genomic DNA of the canarypox virus and with the following oligonucleotides:

| | |
|---|---|
| C6C1 (72 mer) (SEQ ID NO: 22): | 5′CCCGGGCTGCAGCTCGAGGAATTCTTTTTATTGATTAACTAGTCAAATGAGTATAT |
| | ATAATTGAAAAAGTAA3′ |
| and C6D1 (45 mer) (SEQ ID NO: 23): | 5′GATGATGGTACCTTCATAAATACAAGTTTGATTAAACTTAAGTTG3′ |

to isolate a 1210 bp PCR fragment (fragment E).

Fragments D and E were hybridized together to serve as a matrix for a PCR reaction performed with the oligonucleotides C6A1 (SEQ ID NO: 20) and C6D1 (SEQ ID NO: 23) to generate a 1630 bp PCR fragment. This fragment was digested by the SacI and KpnI restriction enzymes to isolate, after agarose gel electrophoresis, a 1613 bp SacI-KpnI fragment. This fragment was ligated with the pBlueScript® II SK+vector (Stratagene, La Jolla, Calif., USA, Cat # 212205) previously digested by the Sacd and KpnI restriction enzymes to give the plasmid pC6L. The sequence of this plasmid was verified by sequencing. The plasmid contains 370 bp of sequences upstream of ORF C6L (C6 left flanking arm), an early transcription stop vaccinia signal, stop codons in the six reading frames, a multiple cloning site containing the Smal, PstI, XhoI and EcoRi restriction sites and finally 1156 bp of sequences downstream of the ORF C6L (C6 right flanking arm).

Plasmid pMPIVC (Schmitt J. F. C. et al., J. Virol., 1988, 62, 1889-1897, Saiki R. K. et al., Science, 1988, 239, 487-491) was used as the matrix for amplifying the complete sequence of the I3L vaccine promoter with the following oligonucleotides:

| | |
|---|---|
| FC112 (33 mer) (SEQ ID NO: 24): | 5′AAACCCGGGCGGTGGTTTGCGATTCCGAAATCT3′ |
| and FC113 (43 mer) (SEQ ID NO: 25): | 5′AAAAGAATTCGGATCCGATTAAACCTAAATAATTGTACTTTGT3′ |

to amplify a 151 bp PCR fragment. This fragment was digested by the Smal and EcoRI restriction enzymes in order to isolate, following agarose gel electrophoresis, an approximately 136 bp Smal-EcoRI restriction fragment. This fragment was then ligated with plasmid pC6L previously digested by SmaI and EcoRI to give the plasmid pFC113.

### Example 16

### Construction of Recombinant Viruses vCP1717 and vCP1718

A PCR reaction was performed using the plasmid pFC106 (Example 8) as the matrix and the following oligonucleotides:

| | |
|---|---|
| FC114 (33 mer) (SEQ ID NO: 26): | 5′TTTCACGTGATGTATAATGCTGATATGATTGAC3′ |
| and FC115 (42 mer) (SEQ ID NO: 27): | 5′TTTTGGATCCGCGGCCGCTTAACGTTTTCCCGAGGCGAAGTC3′ |

to amplify an approximately 2973 bp PCR fragment. This fragment was digested with the PmlI and BamHI restriction enzymes to isolate, following agarose gel electrophoresis, the approximately 2958 bp PmlI-BamHI restriction fragment (fragment F). Plasmid pFC113 (example 15) was digested by the PmlI and BamHI restriction enzymes to isolate, following agarose gel electrophoresis, the approximately 4500 bp PmlI-BamHI restriction fragment (fragment G). Fragments F and G were then ligated together to give the plasmid pFC114.

Plasmid pFC114 was linearized by NotI, then transfected in primary chicken embryo cells infected with canarypox virus vCP1713 (Example 11) according to the previously described calcium phosphate precipitation method (Panicali et Paoletti Proc. Nat. Acad. Sci. 1982, 79, 4927-4931; Piccini et al. In Methods in Enzymology, 1987, 153, 545-563, publishers Wu R. and Grossman L. Academic Press). Positive plaques were selected on the basis of hybridization with a radioactively labelled probe specific to the nucleotide sequence of envelope glycoprotein E NS2A-NS2B. Four successive selection/purification cycles were performed until a pure population was isolated. A representative plaque corresponding to in vitro recombination between the donor plasmid pFC114 and the genome of the ALVAC canarypox virus was then amplified and the recombinant virus stock obtained was designated vCP1717, ALVAC C5 H6p WNV prM-M-E/C6 I3Lp WNV NS2A-NS2B-NS3.

The NotI-linearized pFC114 plasmid was also used for transfecting primary chicken embryo cells infected with the vCP1712 canarypox virus (Example 10) using the procedure described herein. The thus obtained recombinant virus stock was designated vCP1718, ALVAC C5 H6p WNV prM-M-E/C6 I3Lp WNV NS2A-NS2B-NS3.

### Example 17

### Construction of Plasmid pFC115

The complementary DNA (cDNA) of the West Nile fever virus NY99 was synthesized with Gene Amp RNA PCR Kit (Cat # N 808 0017, Perkin-Elmer, Norwalk, Conn. 06859, USA) using the conditions provided by the supplier.

A reverse transcriptase polymerase chain reaction (RT-PCR reaction) was carried out with 50 μl of viral RNA suspension of the West Nile fever virus NY99 (Example 2) and with the following oligonucleotides:

| | |
|---|---|
| FC116 (39 mer) (SEQ ID NO: 28) | 5′TTTTTTGATATCATGACCGGAATTGCAGTCATGATTGGC3′ |
| and FC106 (33 mer) (SEQ ID NO: 8). | |

This pair of oligonucleotides makes it possible to incorporate an EcoRV restriction site, a XbaI restriction site, an initiator code at 5′ and a stop code at 3′ of the insert.

Synthesis of the first cDNA strand takes place by elongation of the oligonucleotide FC106, following its hybridization with the RNA matrix.

The synthesis conditions of the first cDNA strand were a temperature of 42° C. for 15 min, then 99° C. for 5 min and finally 4° C. for 5 min. The conditions of the PCR reaction in the presence of the pair of oligonucleotides FC106 and FC116 were a temperature of 95° C. for 2 min, then 35 cycles (95° C. for 1 min, 62° C. for 1 min and then 72° C. for 2 min) and finally 72° C. for 7 min to produce a 2079 bp fragment.

This fragment was digested by EcoRV and then XbaI to isolate, following agarose gel electrophoresis, the approximately 2061 bp EcoRV-XbaI fragment.

This fragment was ligated with the pVR1012 expression plasmid previously digested by XbaI and EcoRV to give the plasmid pFC115 (6956 bp). Under the control of the early human cytomegalovirus promoter or hCMV-IE (human Cytomegalovirus Immediate Early), this plasmid contains an insert encoding the polyprotein prM-M-E.

### Example 18

### Construction of the Recombinant Viruses vCP2017-H

A PCR reaction was carried out using the plasmid pFC115 (Example 17) as the matrix and the following oligonucleotides:

| | |
|---|---|
| FC117 (36 mer) (SEQ ID NO: 29): | 5′TTTTCGCGAATGACCGGAATTGCAGTCATGATTGGC3′ |
| and FC111 (39 mer) (SEQ ID NO: 19) | |

to amplify an approximately 2082 bp PCR fragment. This fragment was digested by NruI and SalI restriction enzymes to isolate, after agarose gel electrophoresis, an approximately 2071 bp NrI-SalI restriction fragment. This fragment was then ligated with plasmid pFC107 (Example 9) previously digested by the NruI and SalI restriction enzymes to give the plasmid pFC116.

Plasmid pFC116 was linearized by NotI and then transfected in primary chicken embryo cells infected with canarypox virus (ALVAC strain) using the procedure of Example 10. A representative plaque corresponding to in vitro recombination between the donor plasmid pFC116 and the genome of the ALVAC canarypox virus was selected on the basis of a hybridization with a radioactively labelled probe specific to the nucleotide sequence of the envelope glycoprotein E and was then amplified. The recombinant virus stock obtained was designed vCP2017-H.

### Example 18.1

### Construction of a C5 H6p WNV prM-M-E Donor Plasmid (pDS-2946-1-1) for the Generation of ALVAC WNV (vCP2017) or ALVAC-2 WNV (vCP2018).

The construction scheme is illustrated in FIG. 1.

A pTriEx-WNV vector containing the 3′-end of the West Nile Virus capsid gene, and the prM/M and E genes with a poly-His tag at the end of the E protein from a NY99-related isolate, was obtained from Cornell University, and ALVAC (containing WNV prM-M-E in C5 locus) is illustrated in FIG. 1. The WNV E gene contains an internal T5NT sequence, which is known to result in premature transcriptional termination in pox-based recombinants (Yuen and Moss, Proc. Natl. Acad. Sci. USA 84:6417-6421, 1987). In order to mutate the T5NT sequence, the 1.4 kb Cla I-Xho I 3′-WNV fragment from pTriEx-WNV was inserted into pUC-4K, generating clone pDS-2889-1, pUC 3′ WNV. Site-directed mutagenesis was performed using the Amersham QuikChange kit and primers 7598.SL (SEQ ID NO: 38) and 7599.SL (SEQ ID NO: 39).

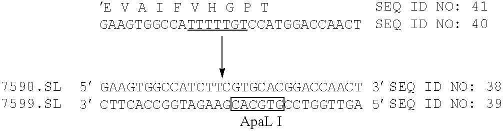

An ApaL I site was introduced for screening purposes. Clone pDS-2897-5-1 (pUC 3′-WNV′T5NT) was confirmed as correct by sequence analysis.

In order to remove the poly-His tag and introduce a translation stop and terminal T5NT, the 1.4 kb 3′ fragment from pDS-2897-5-1 was PCR amplified using primers 7617.SL (SEQ ID NO: 42) and 7601.SL (SEQ ID NO: 43).

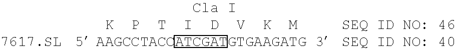

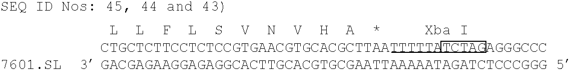

Primer 7601.SL introduces a stop codon, T5NT transcription termination signal, and an Xba I site for cloning. The resultant fragment was inserted into pCR2.1 and clone pDS-2918-1 (pCR2.1 3′-WNV-T5NT+stop) was confirmed as correct by sequence analysis.

The 0.7 kb EcoR V-Cla I 5′-end of the WNV gene cassette was PCR amplified using primers 7600.SL (SEQ ID NO: 47) and 7616.SL (SEQ ID NO: 48) and the fragment inserted into pCR2.1 to generate plasmid pDS-2905-2-1

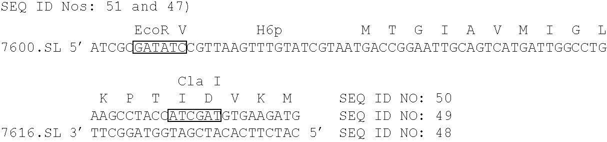

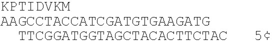

The sequence of the insert was confirmed.

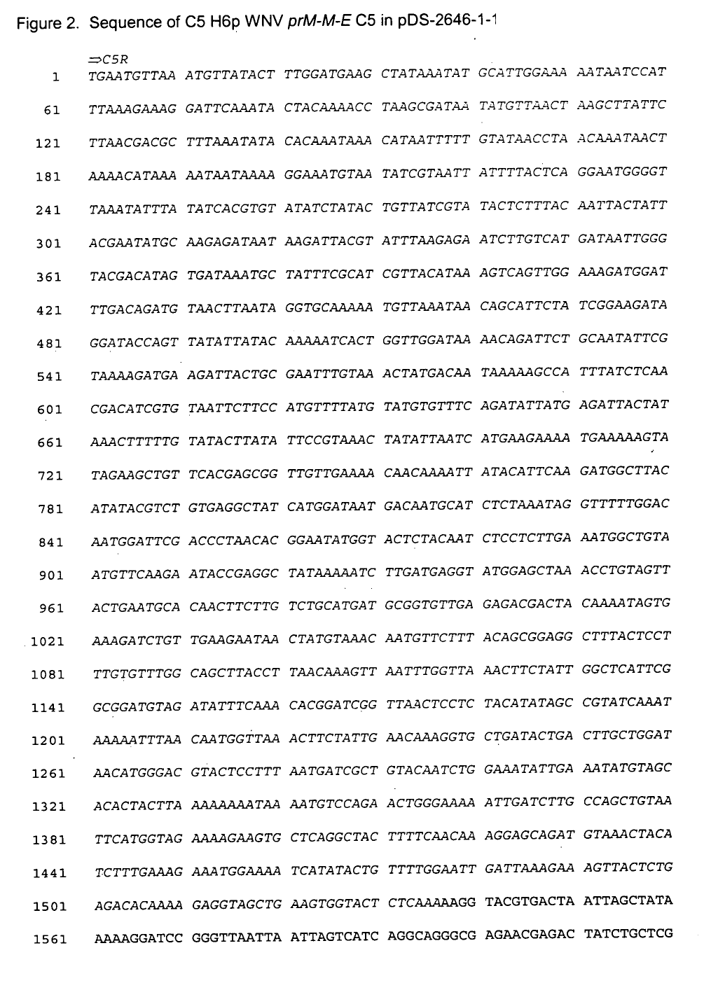

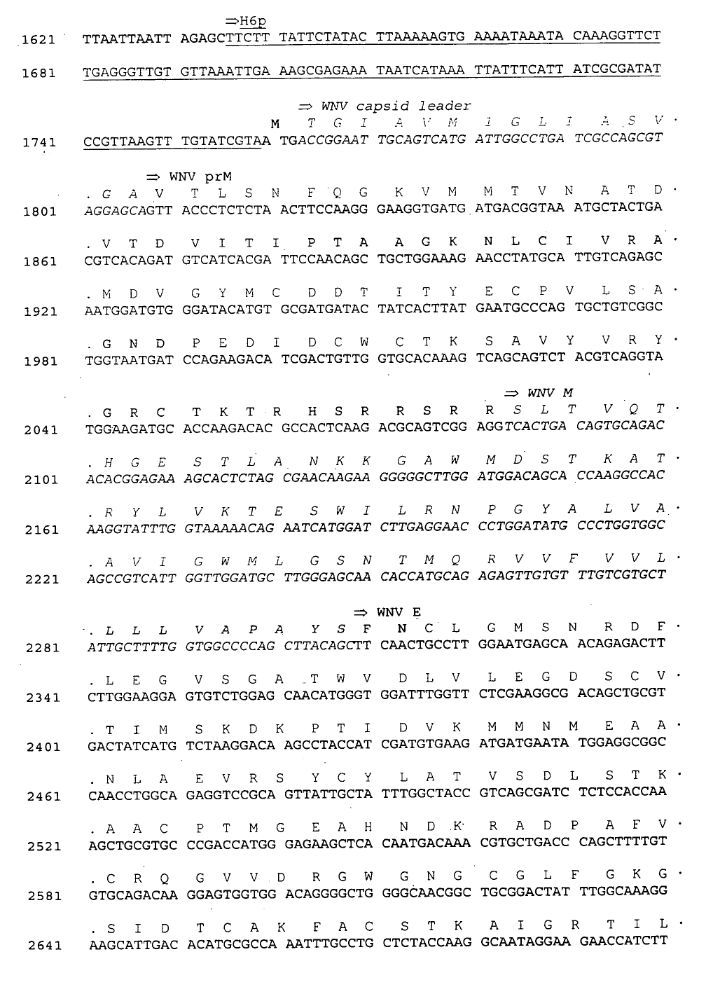

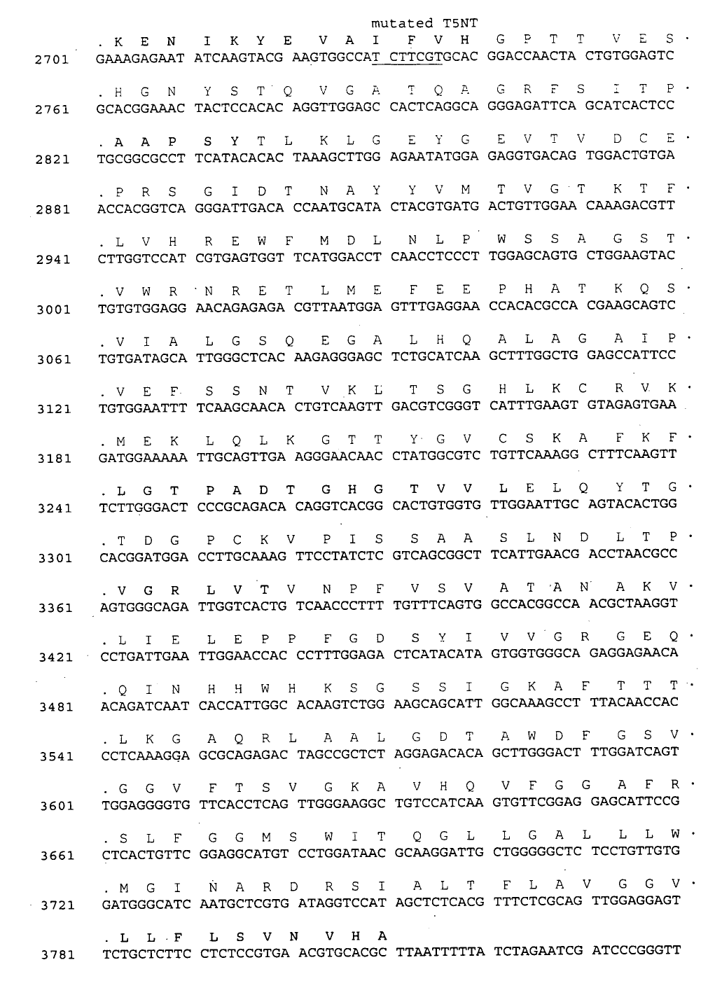

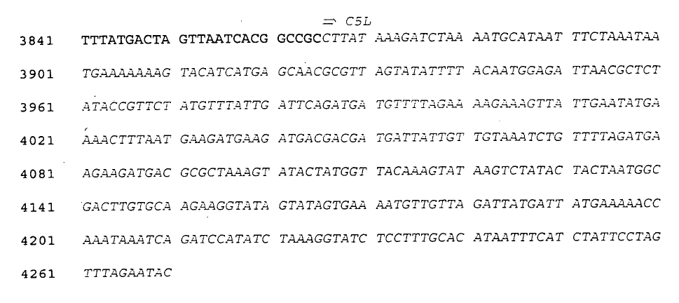

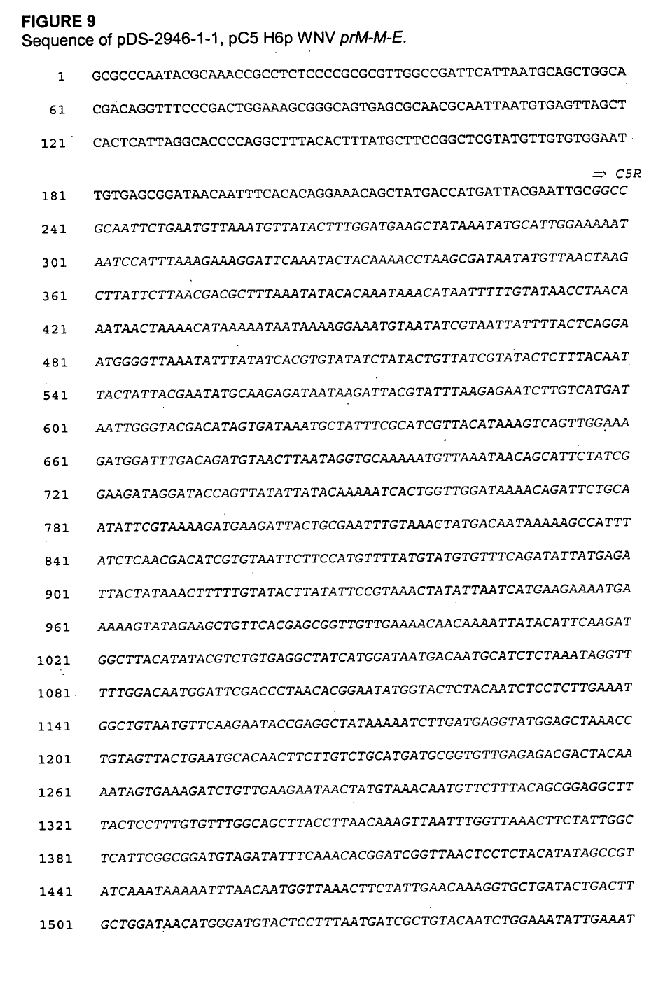

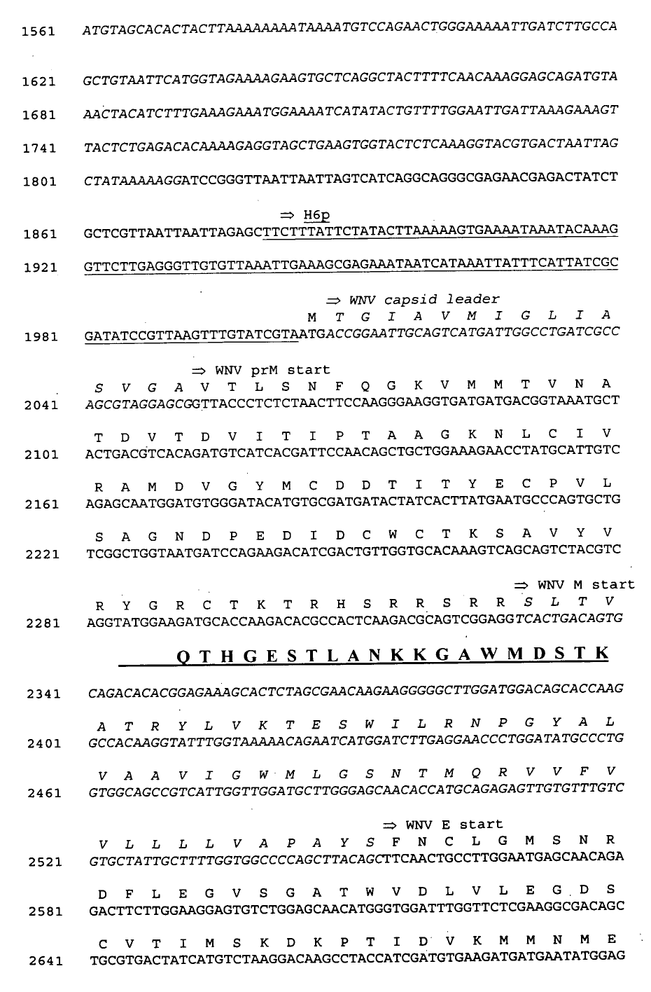

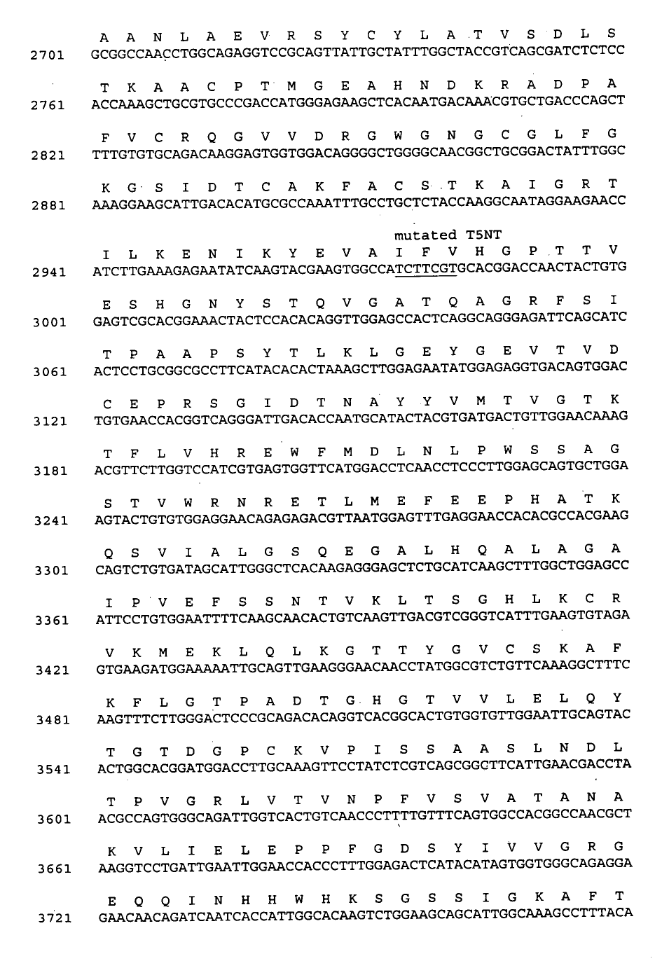

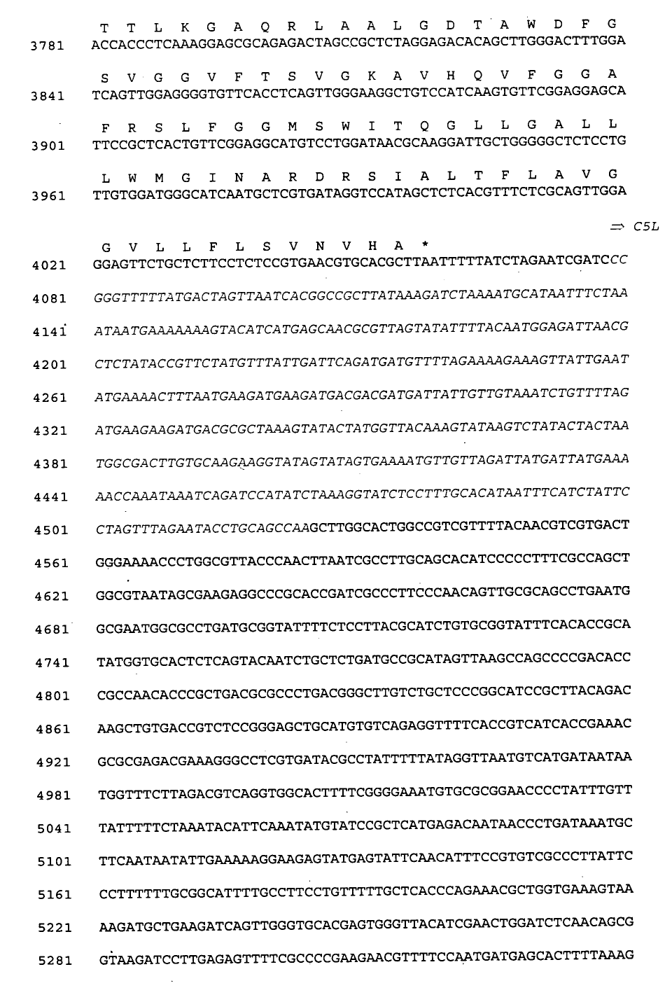

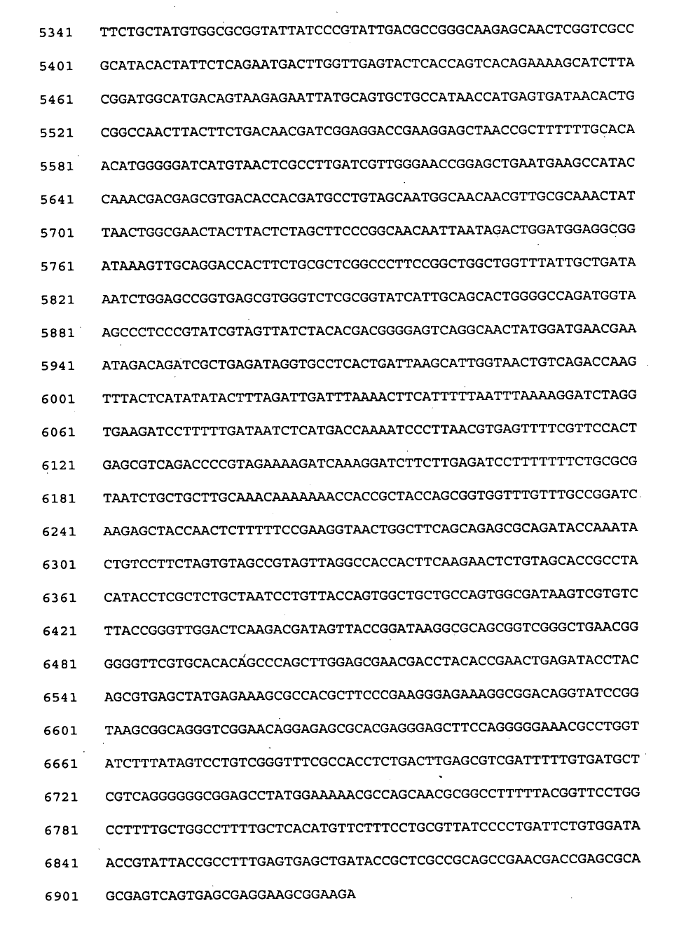

Primer 7600.SL contains the 3′-end of the H6 promoter from the Nrul site and primer 7616.SL spans a Cla I site in the WNV E gene. The EcoR V-Cla I 5′-fragment from pDS-2905-2-1 and the Cla I-Xba I 3′ fragment from pDS-2918-1 were inserted into the ALVAC C5 donor plasmid pNVQH6C5LSP-18 (pC5 H6p donor plasmid) that had been digested with EcoR V and Xba I. Clone pDS-2946-1-1 was confirmed by restriction enzyme analysis and the H6p WNV prM-M-E insert was confirmed by sequence analysis. The sequence of the C5- H6p WNV prM-M-E-C5 gene cassette is illustrated in FIG. 2, and the full sequence is illustrated in FIG. 9. From the sequences depicted in FIG. 2 and FIG. 9, one of skill in the art may clone these sequences and easily generate the plasmids herein without following this exact procedure.

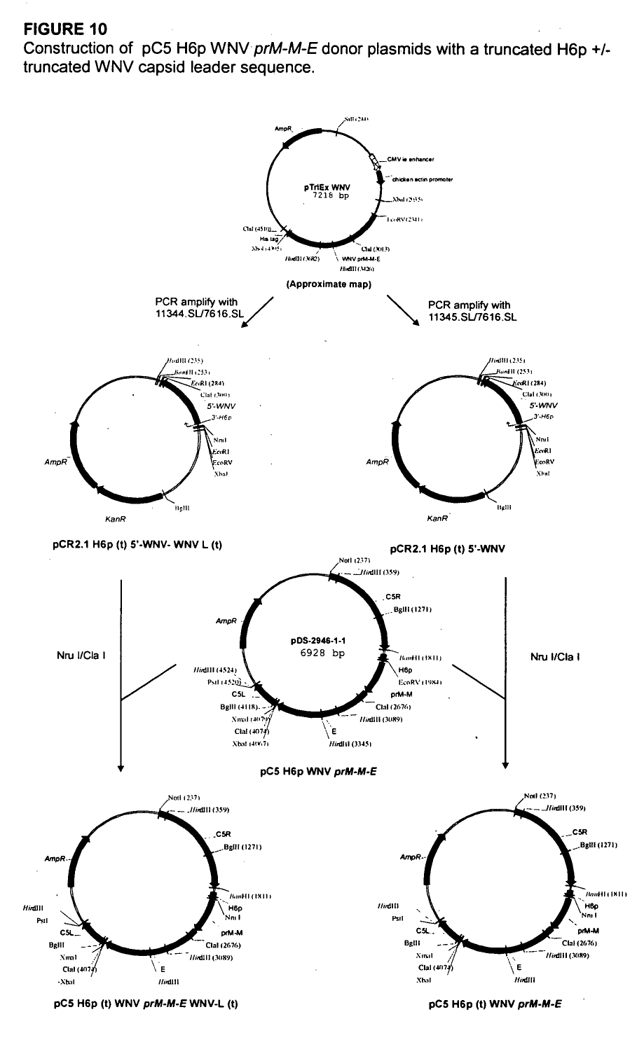

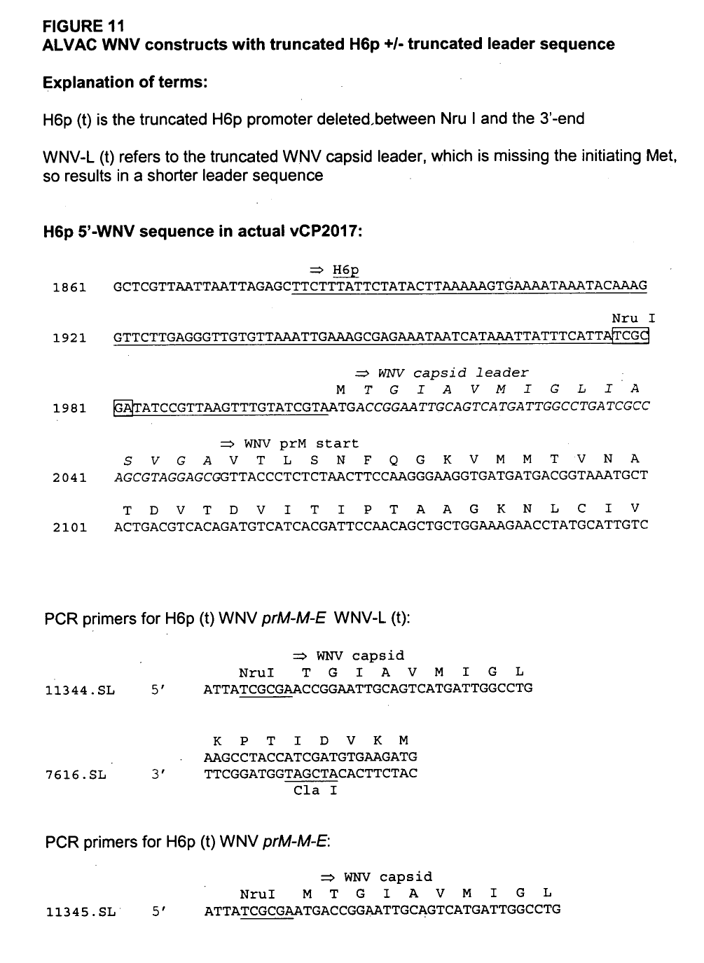

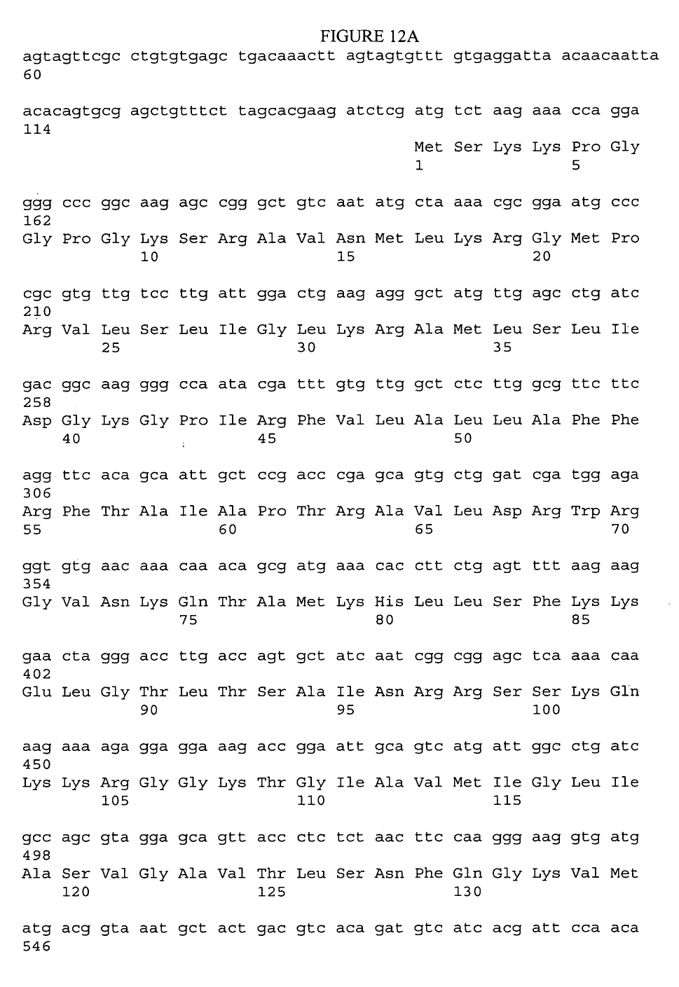

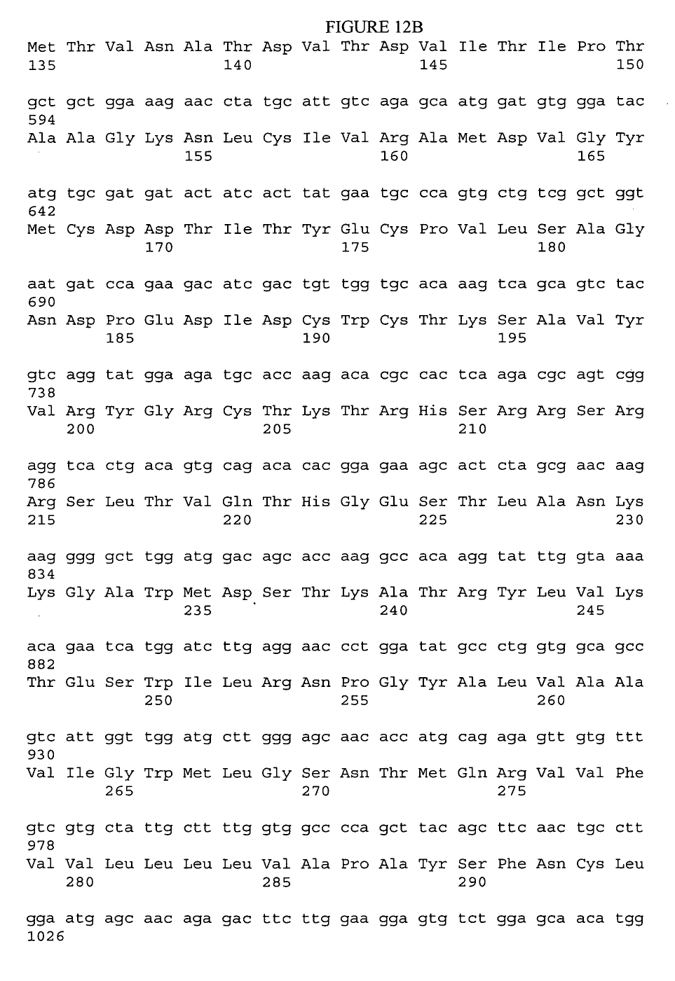

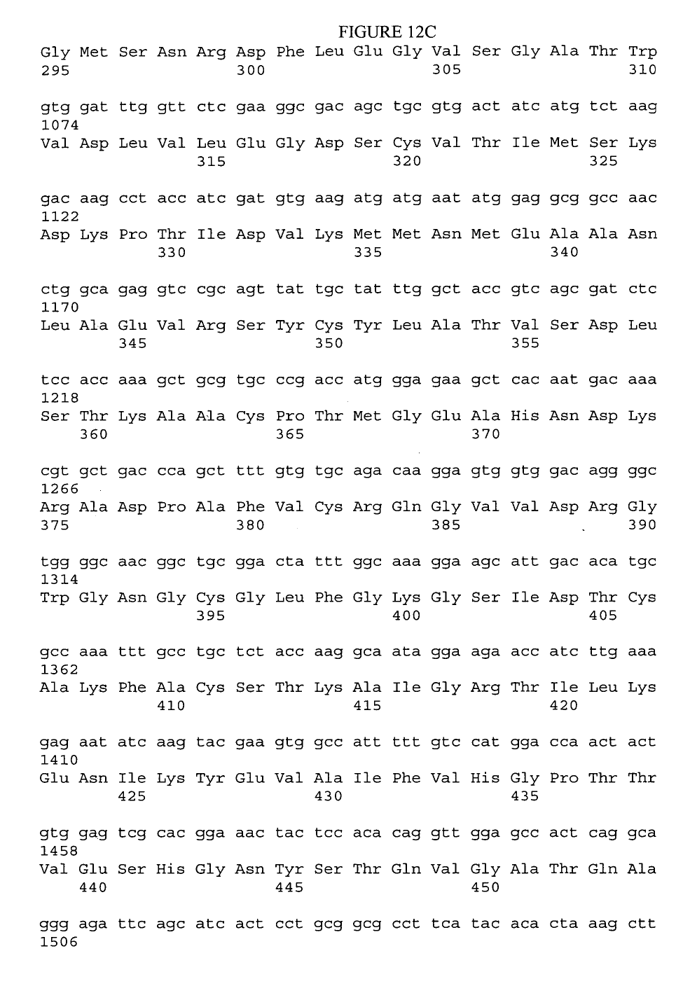

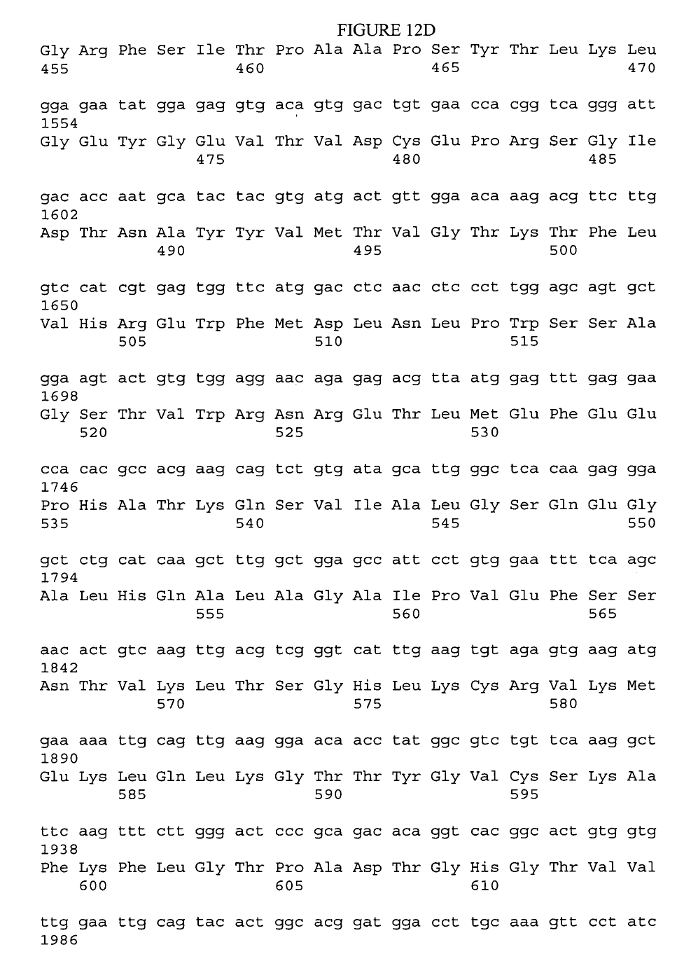

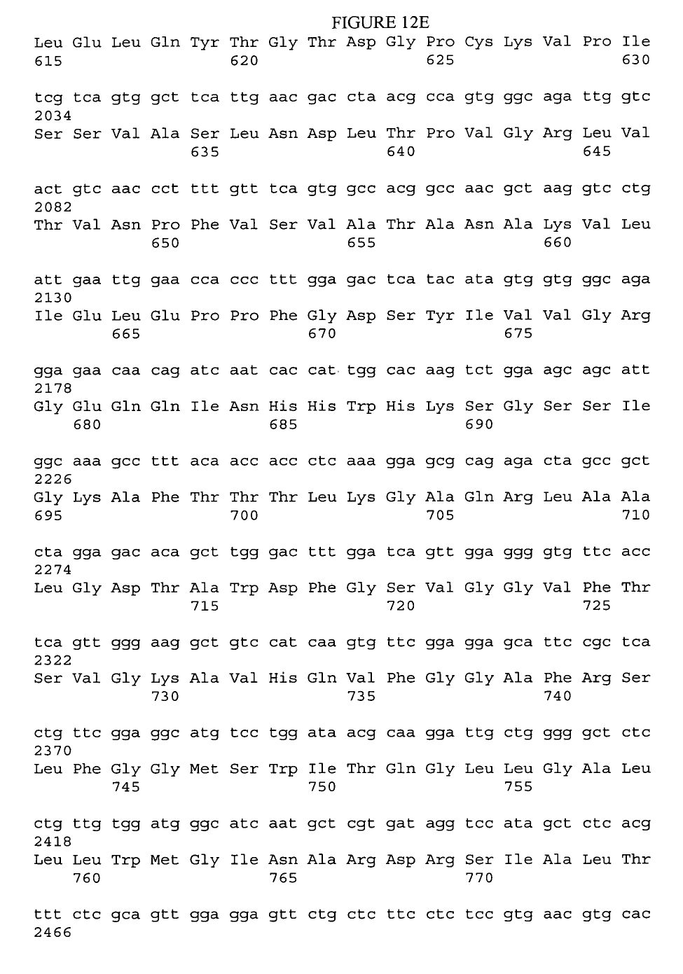

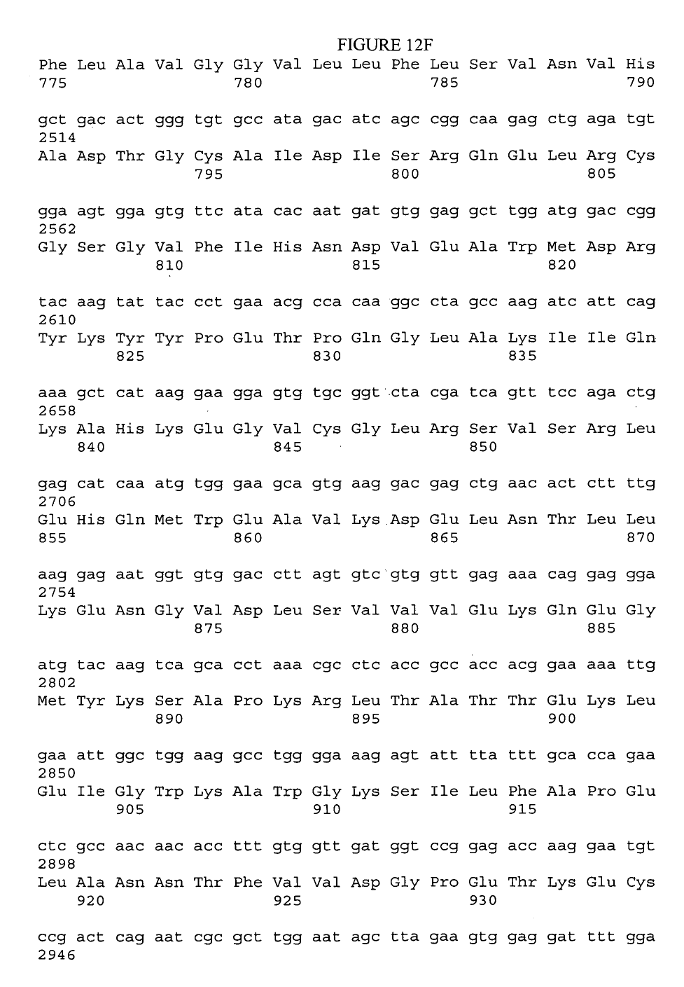

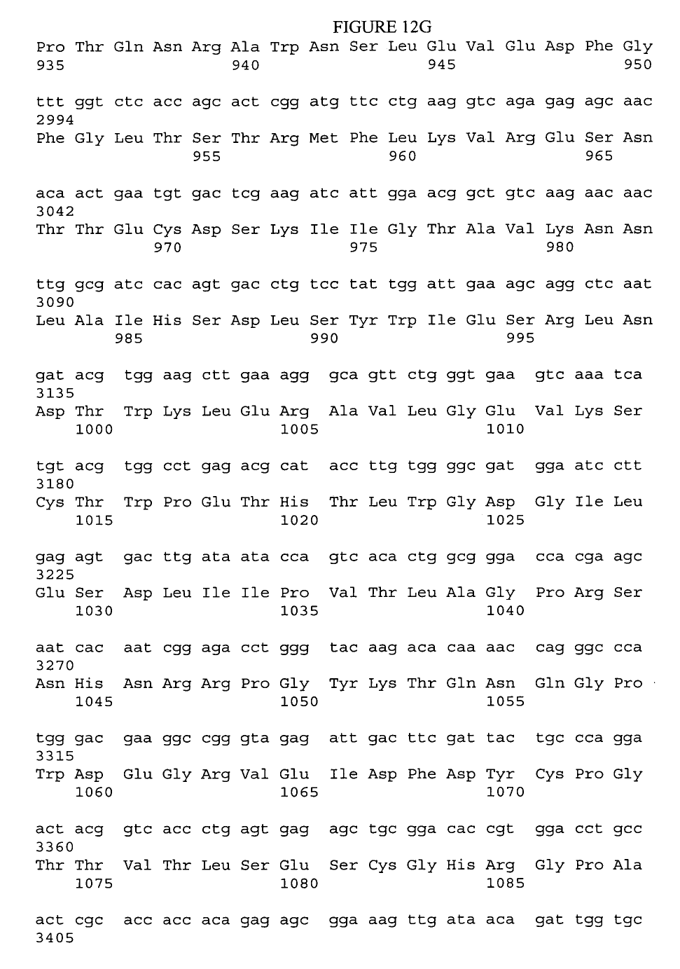

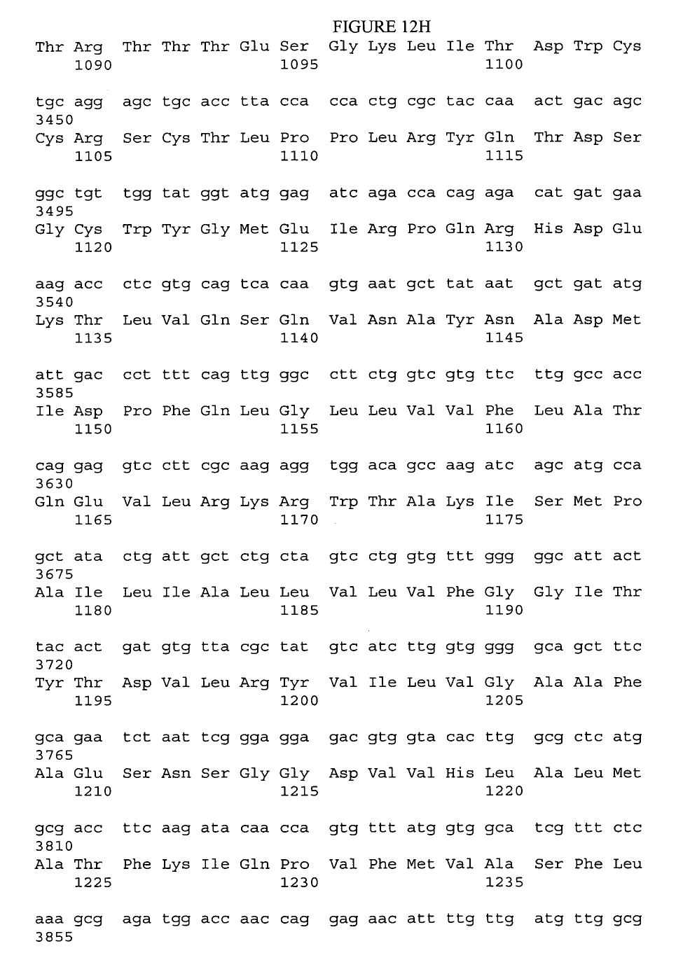

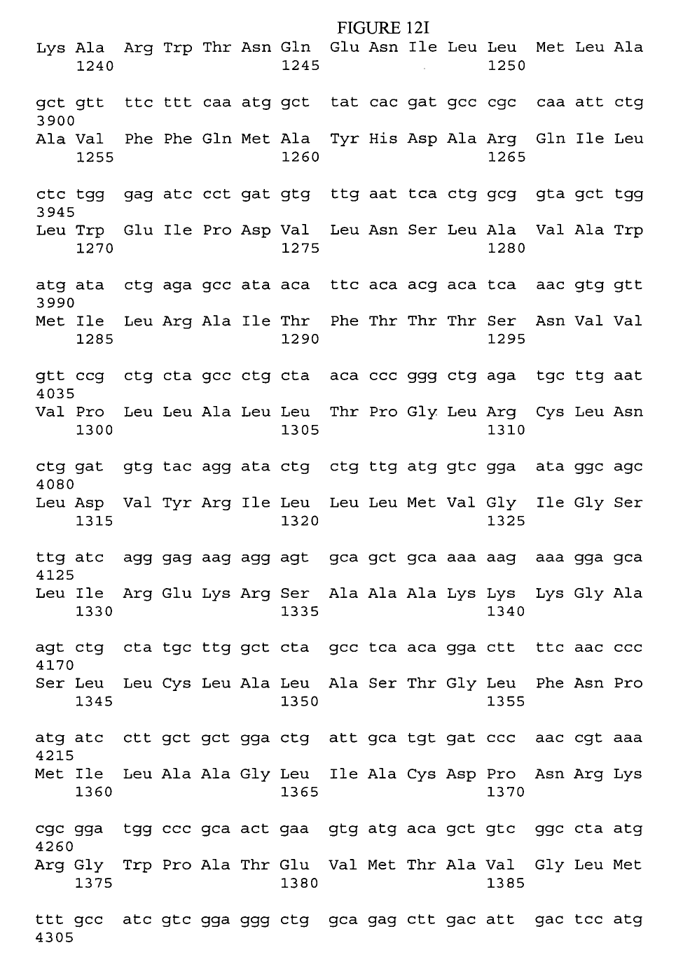

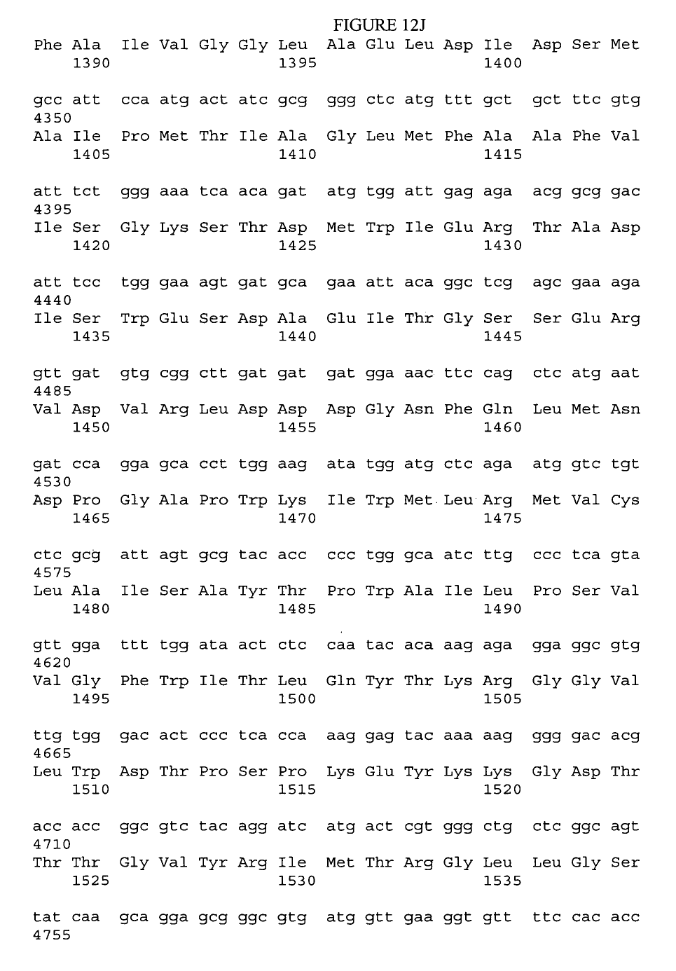

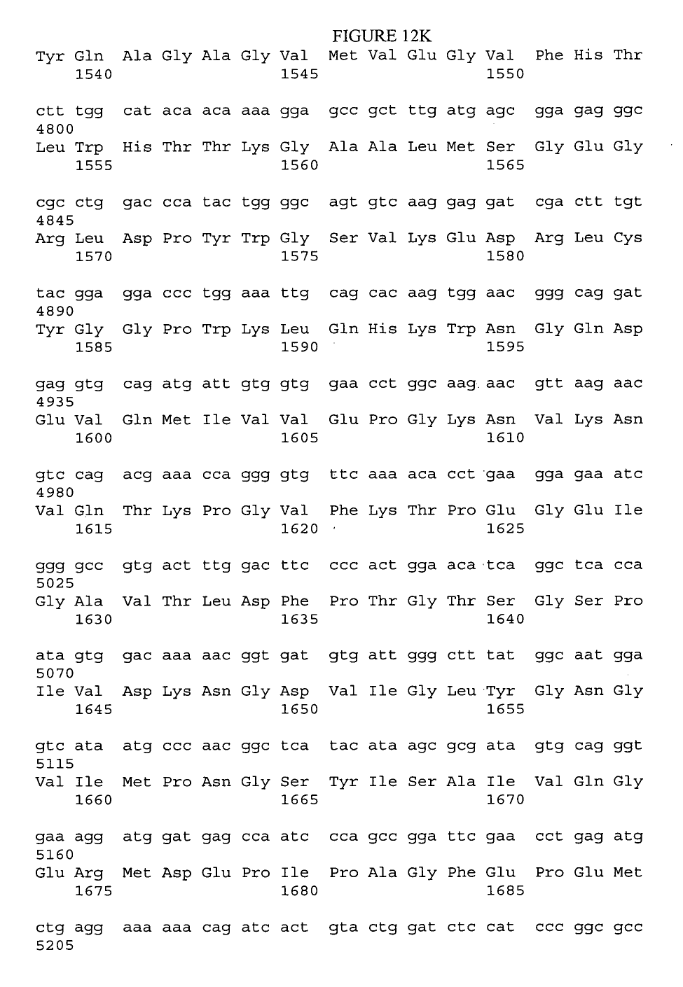

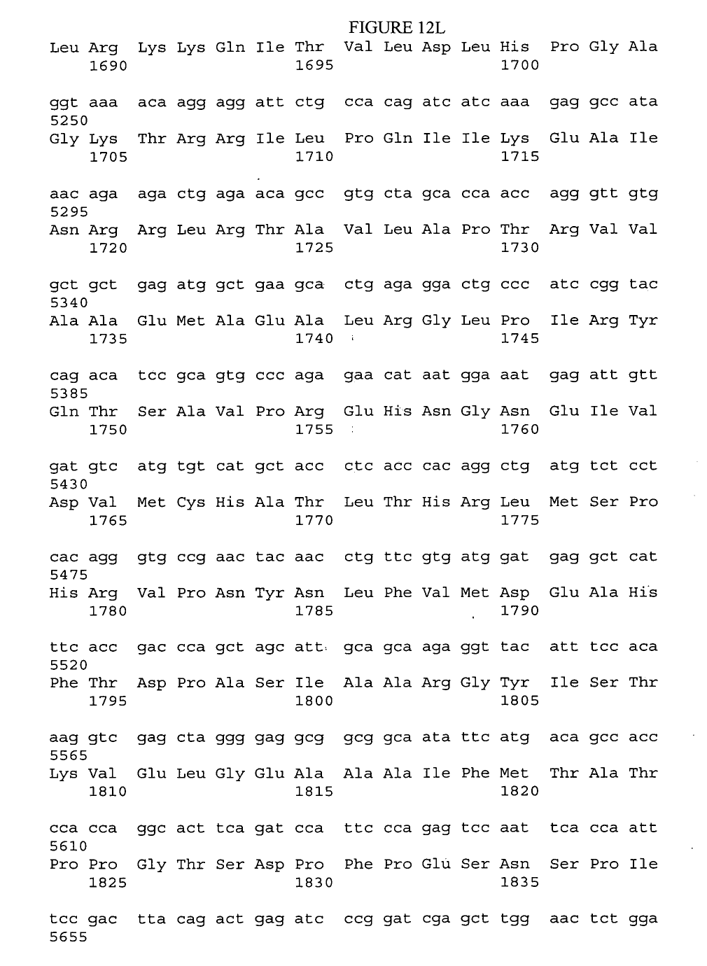

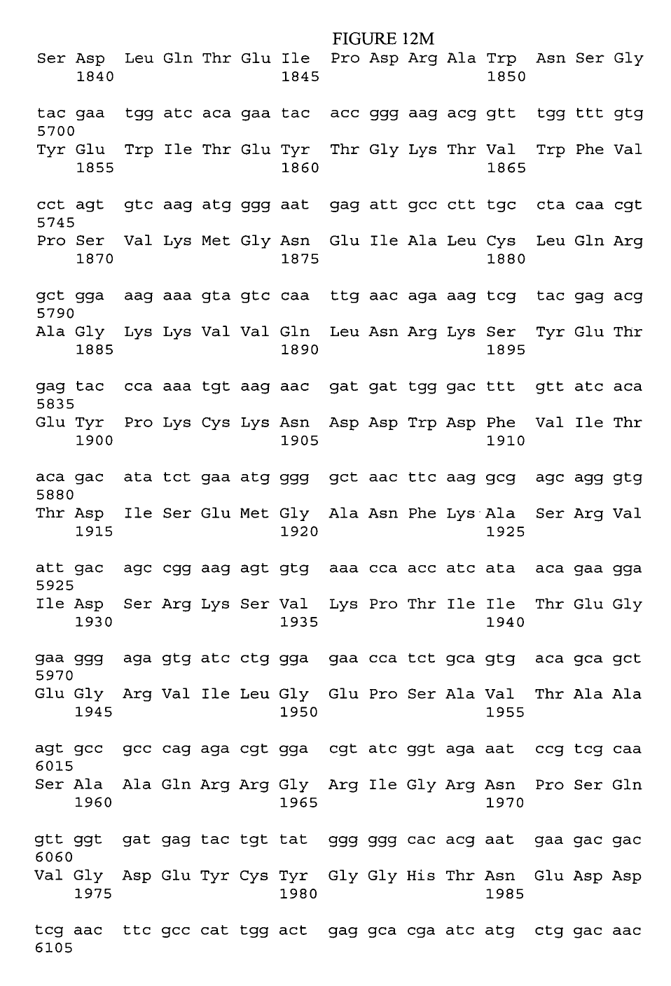

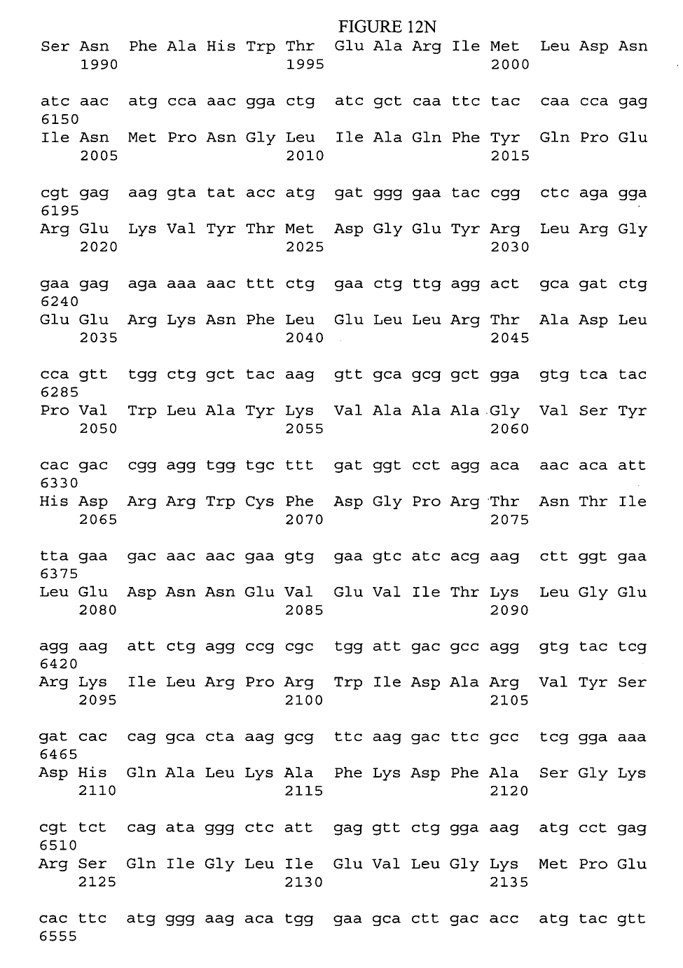

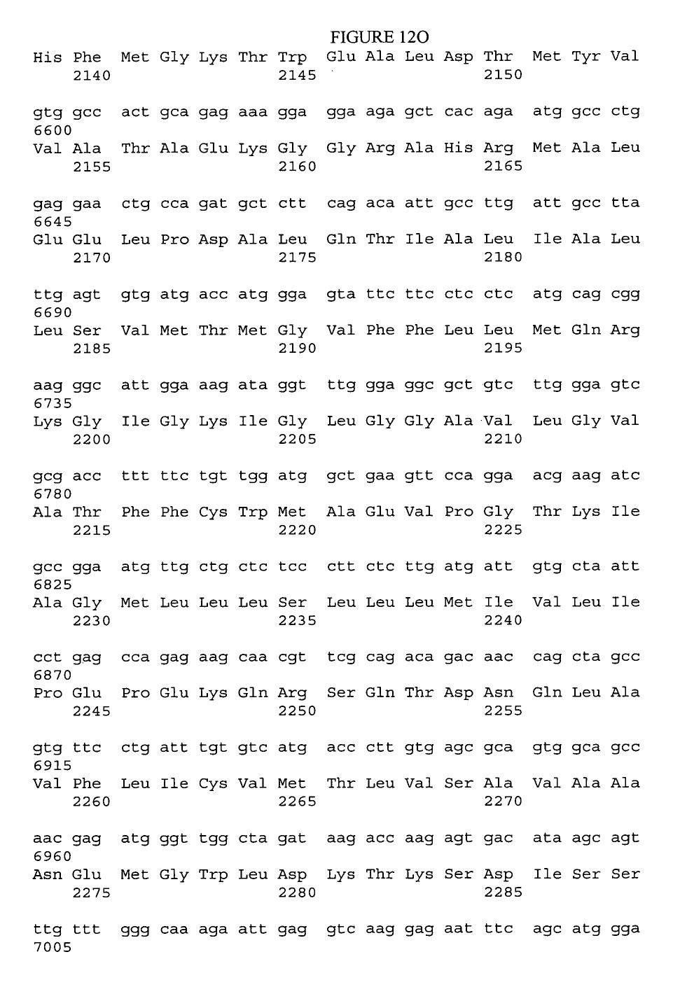

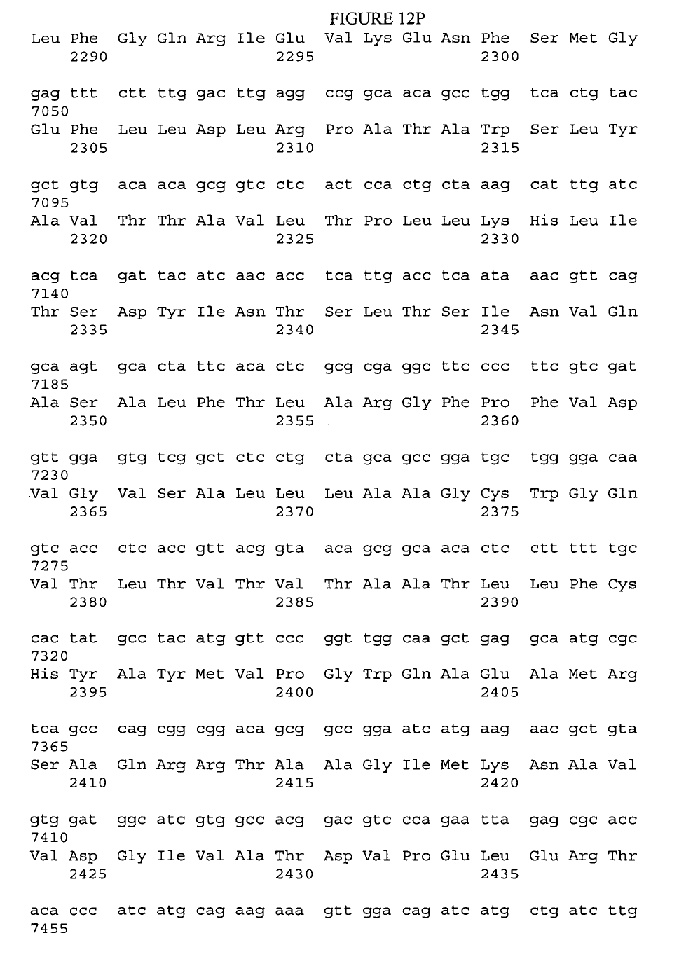

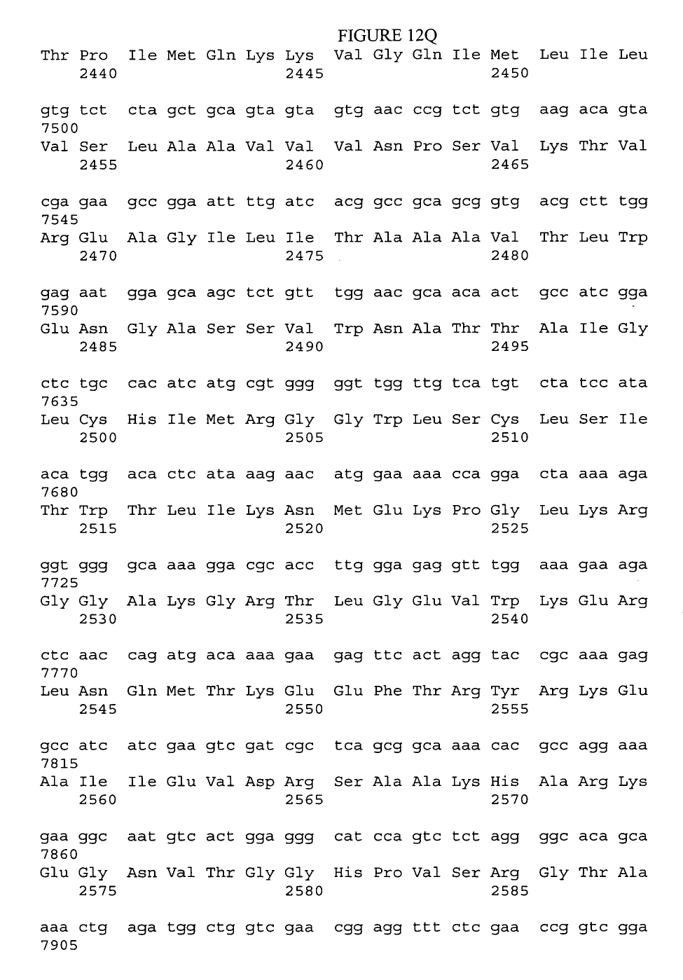

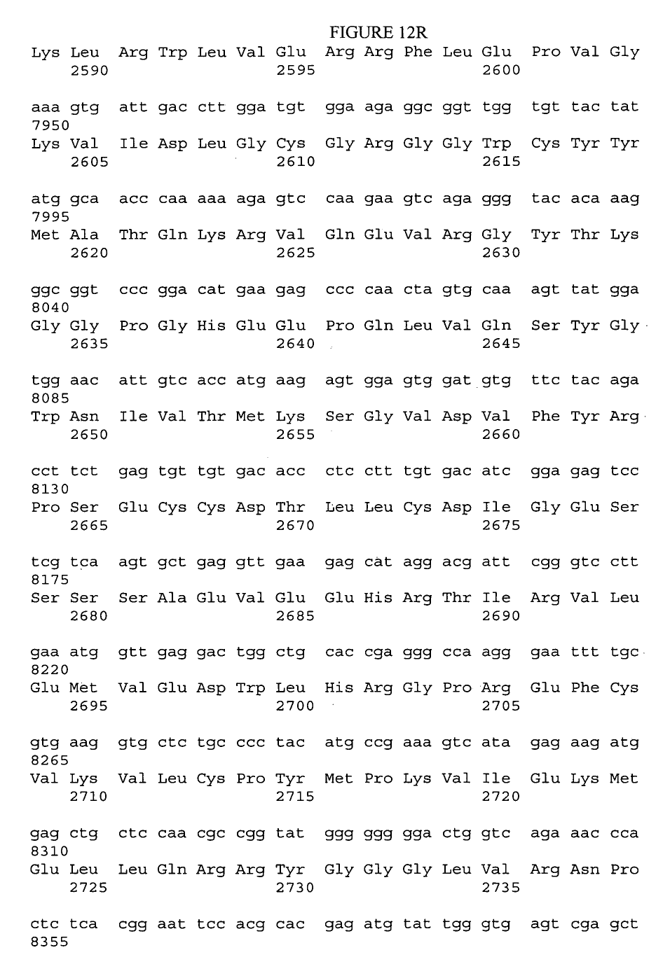

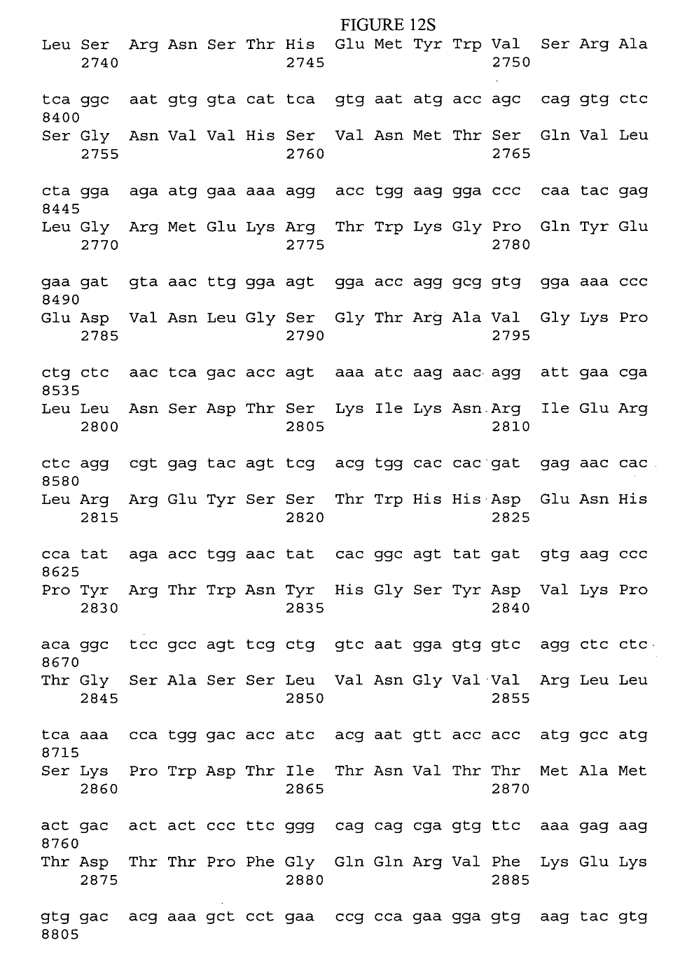

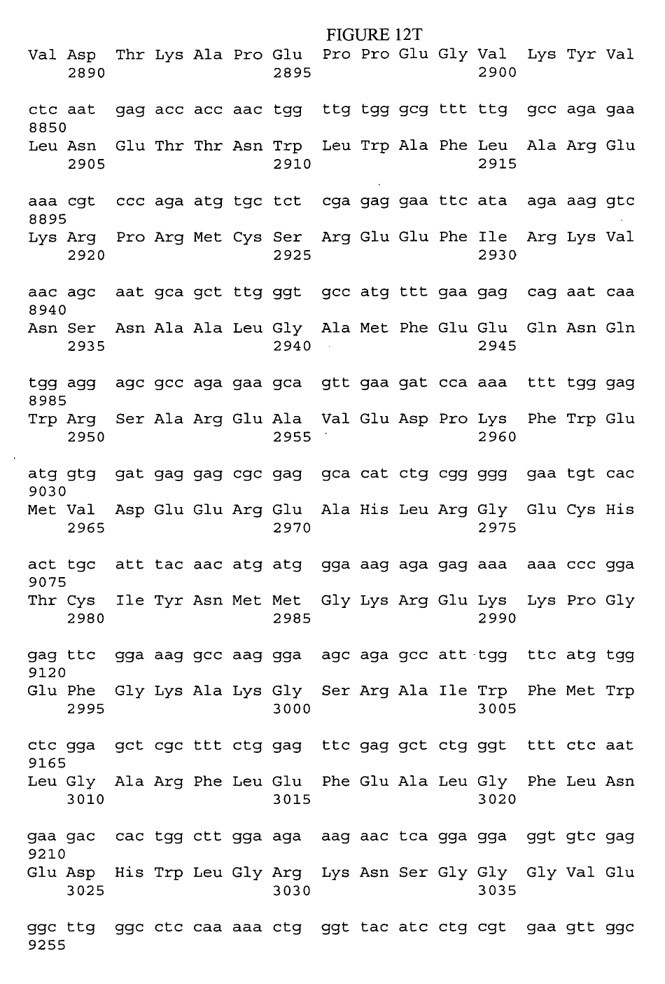

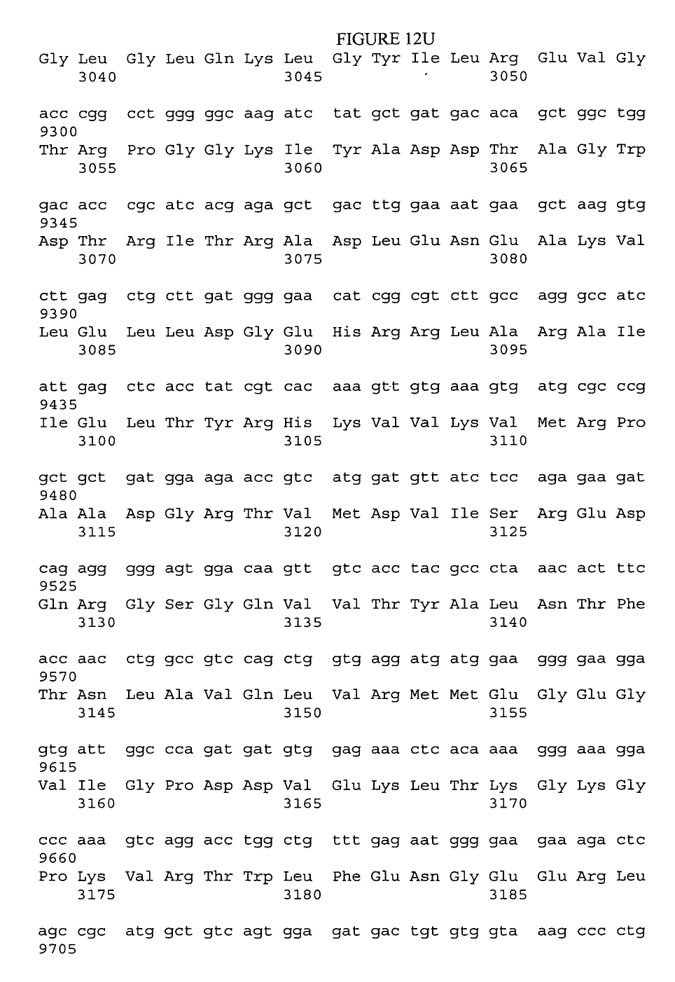

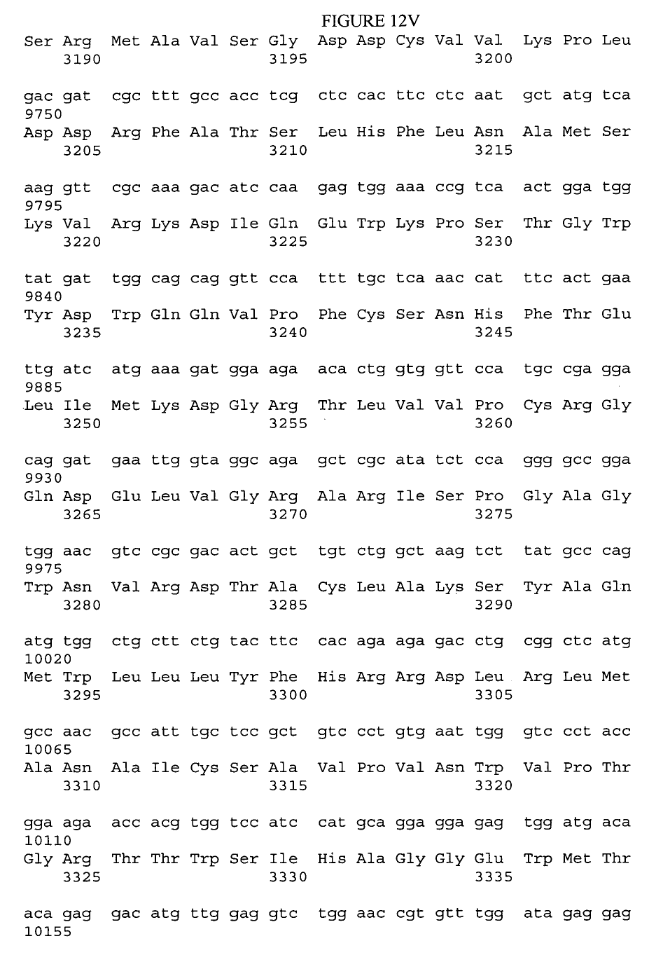

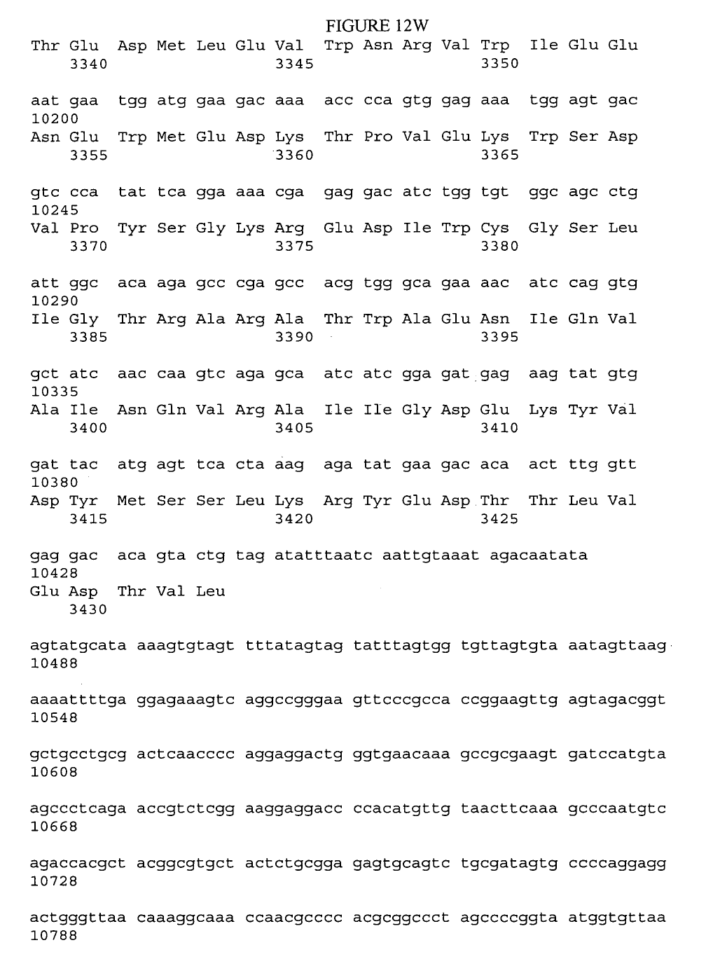

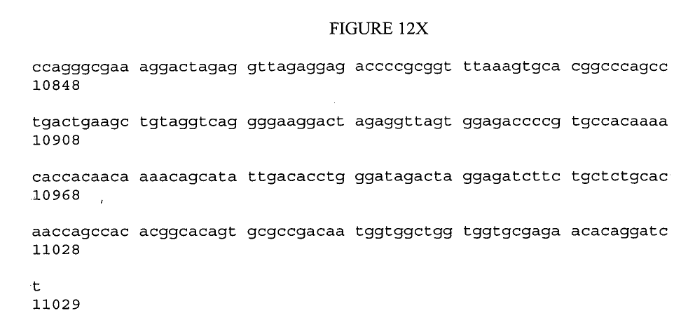

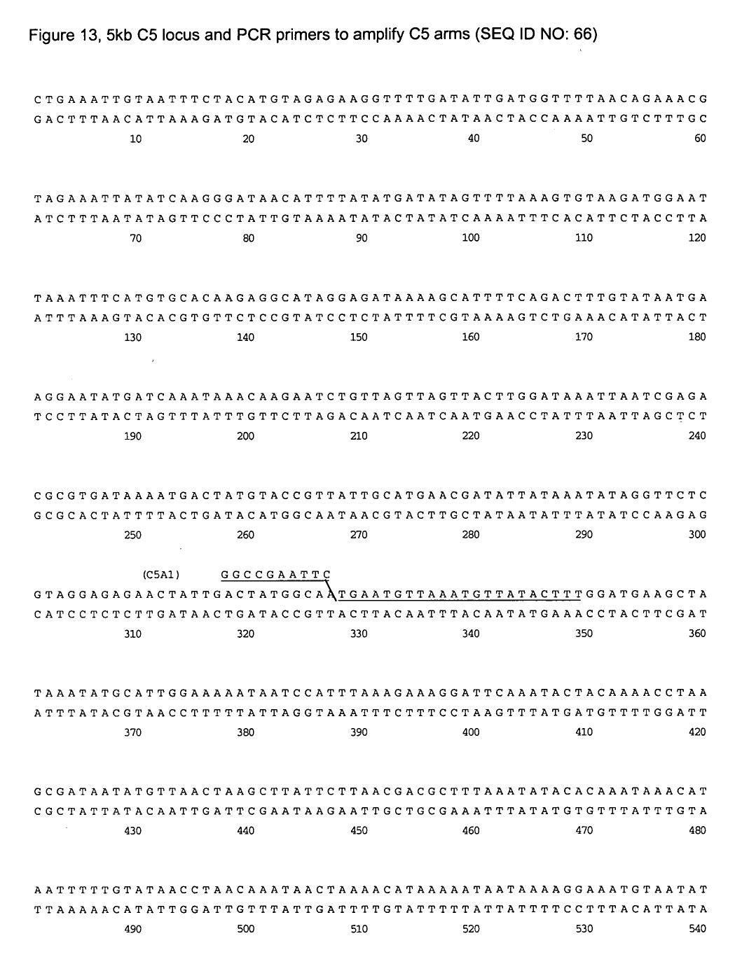

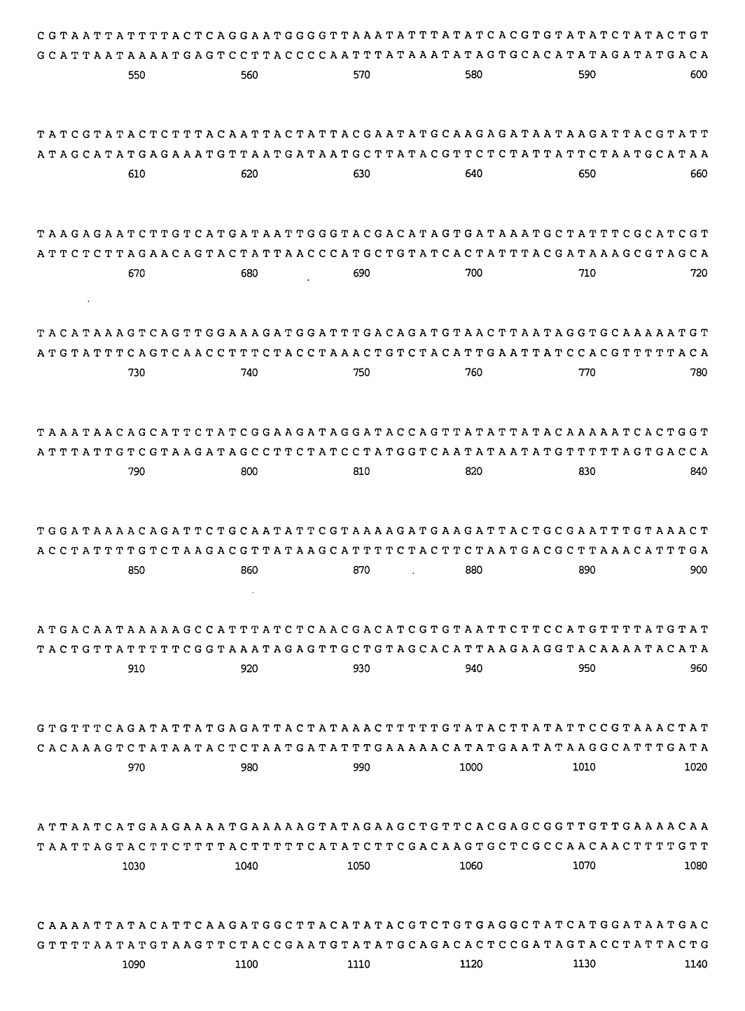

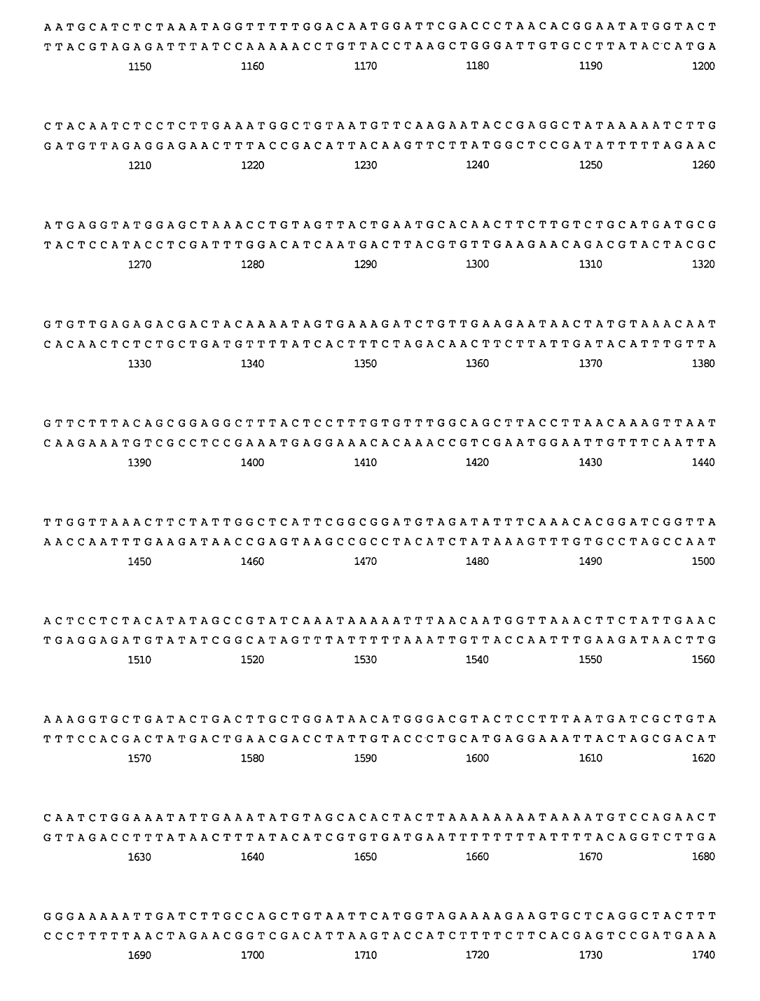

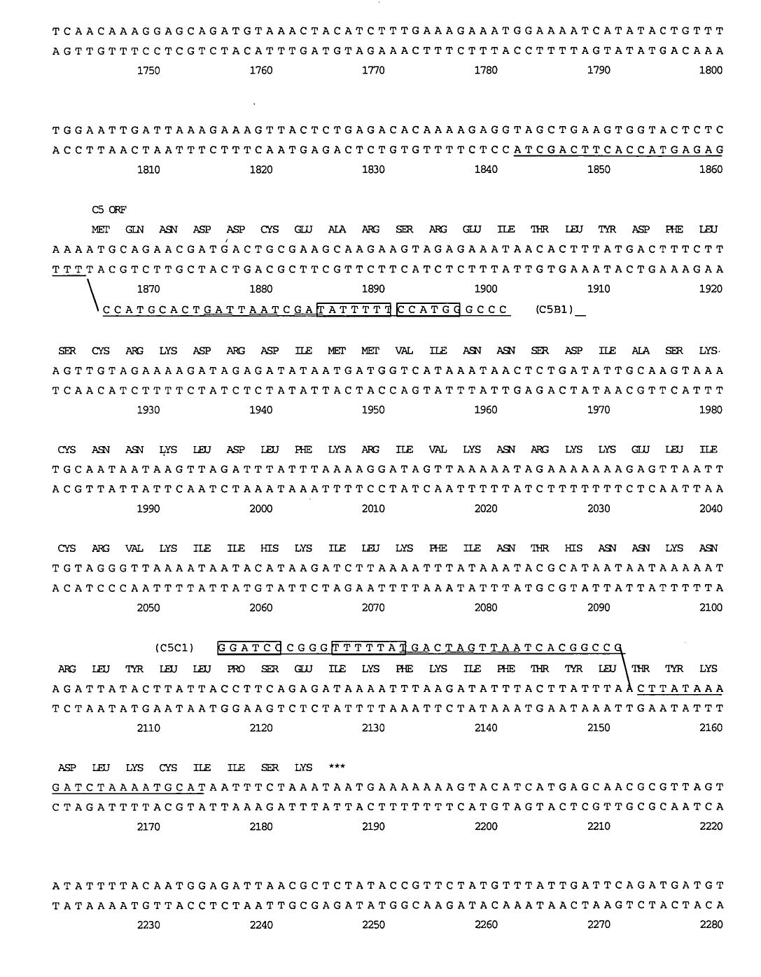

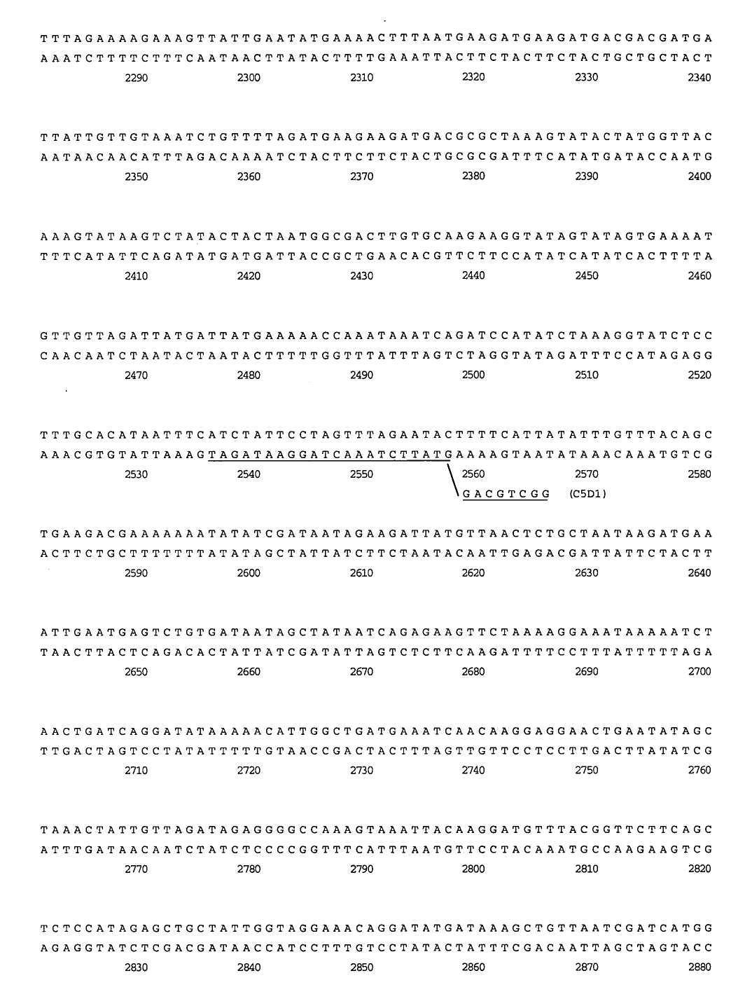

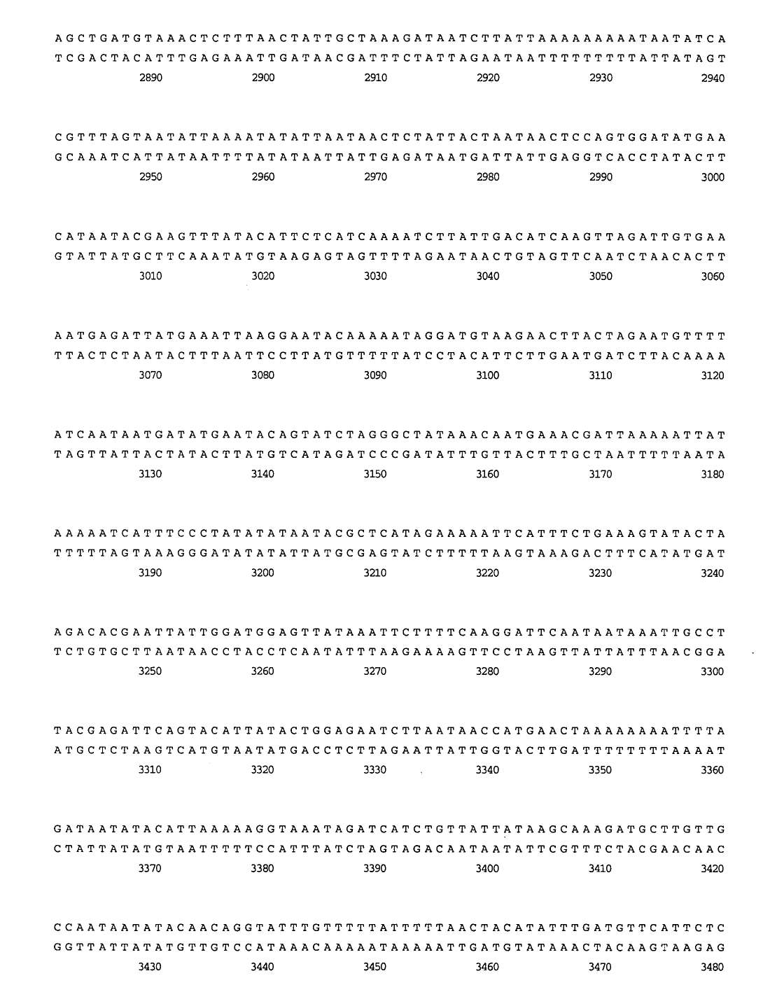

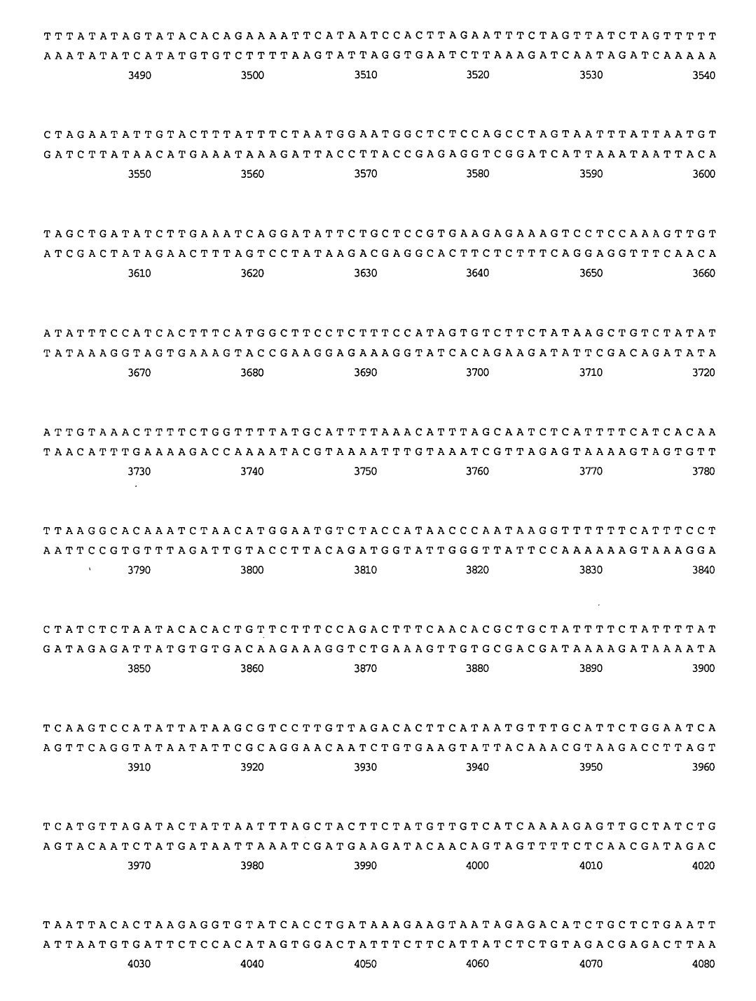

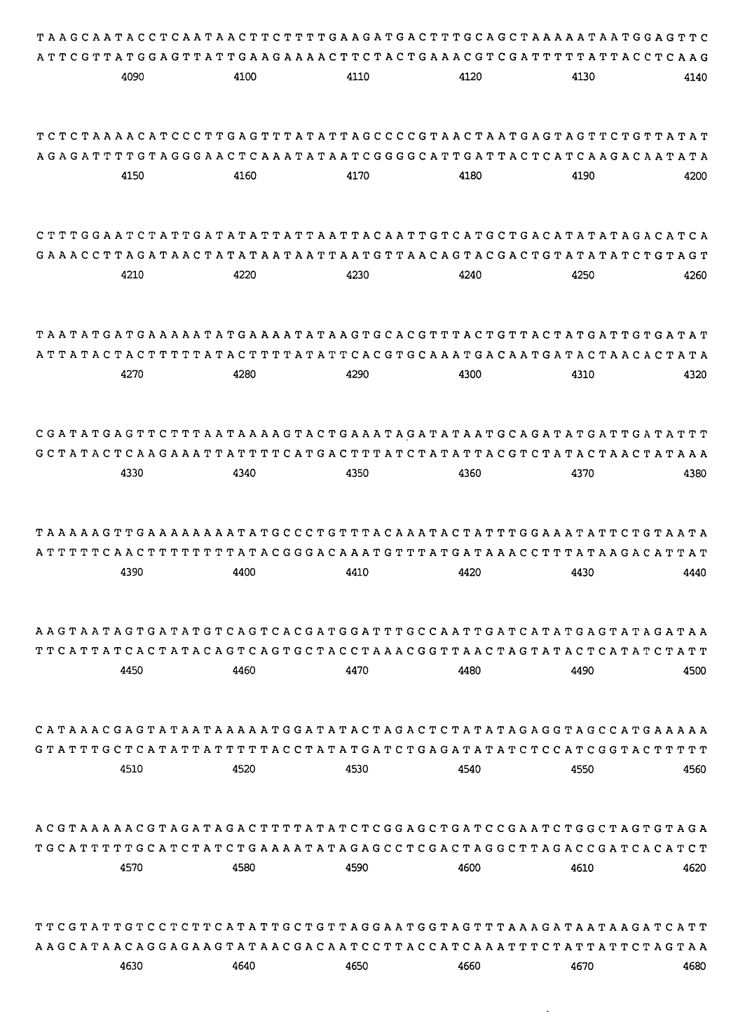

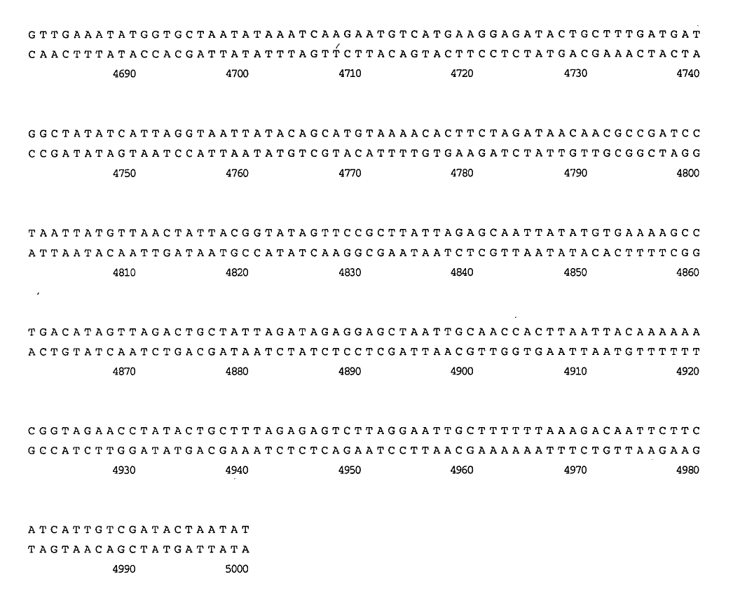

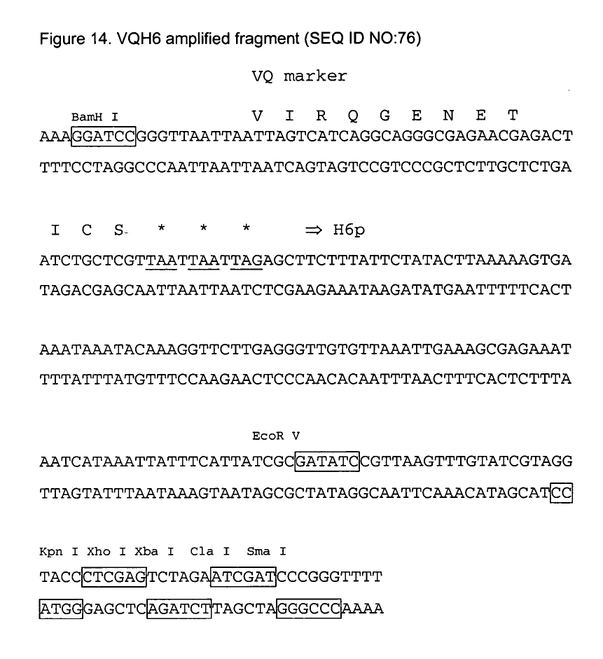

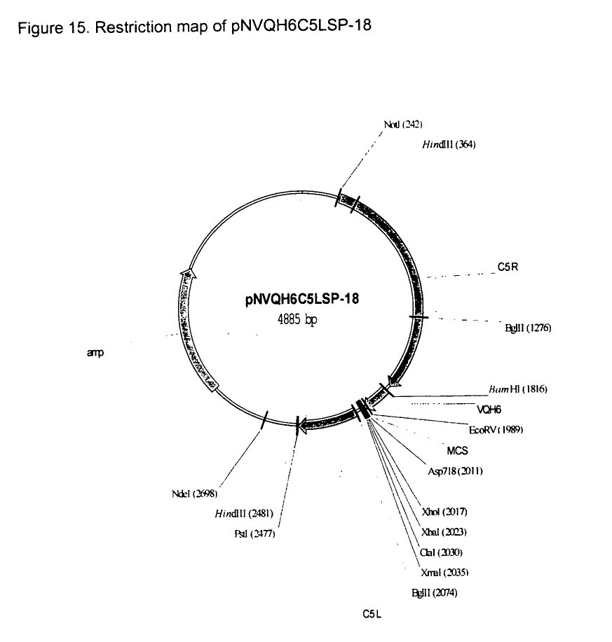

Alternatively, plasmids with a truncated H6p and/or truncated WNV capsid leader sequence may be useful (see FIG. 10 and FIG. 11).

### Example 18.2

### Generation and Characterization of ALVAC WNV, (vCP2017) and ALVAC-2 WNV (vCP2018).

ALVAC-2 is a canarypox virus containing an E3LK3L gene cassette inserted at the unique C6 locus of ALVAC and is described in U.S. Pat. No. 5,756,103. To generate ALVAC- or ALVAC-2-based WNV recombinants, primary chick embryo firbroblast cells (CEFS) were transfected with Not I-linearized pDS-2946-1-1 plasmid DNA (pC5 H6p WNV prM-M-E) mixed with FuGENE-6 transfection reagent (Roche), then infected with ALVAC or ALVAC-2 as rescue virus at an MOI of 10. After 24-48 hours, recombinant plaques were lifted onto nylon membrane and hybridized with a WNV-specific DNA probe which was labelled with horseradish peroxidase according to the manufacturer's protocol (Amersham Cat# RPM3001). Following 2-4 sequential rounds of plaque purification, single plaques were amplified to produce stocks of vCP2017 and vCP2018. Recombinant viruses were characterized by restriction enzyme and Southern blot analyses. The C5-H6p WNV-C5 locus was PCR-amplified and the complete sequence confirmed. Expression of the WNV proteins was confirmed by immunoplaque and immunoblot analyses. Both of these recombinants contain two copies of the H6p WNV prM-M-E genes.

### Example 19

### Production of Recombinant Vaccines

For the preparation of equine vaccines, the recombinant canarypox vCP1712 virus (Example 10) is adjuvanted with carbomer solutions, namely Carbopol™974P manufactured by BF Goodrich, Ohio, USA (molecular weight about 3,000,000).

A 1.5% Carbopol™974P stock solution was initially prepared in distilled water containing 1 g/l of sodium chloride. This stock solution was then used for the preparation of a 4 mg/ml Carbopol™974P solution in physiological salt solution. The stock solution was mixed with the adequate volume of the physiological salt solution, either in a single stage or in several successive stages, the pH value being adjusted in each stage with a IN sodium hydroxide solution (or even more concentrated) in order to obtain a final pH value of 7.3 to 7.4.

The ready-to-use Carbopol™974P solution obtained in this way was used for taking up recombinant, lyophilized viruses or for diluting concentrated, recombinant virus stock solutions. For example, to obtain a viral suspension containing 10⁸ pfu/l ml dose, a viral stock solution was diluted so as to obtain a titer of 10^{8.3} pfu/ml, followed by dilution in equal parts with said ready-to-use 4 mg/ml Carbopol™974P solution.

Recombinant vaccines can also be produced with recombinant canarypox viruses vCP1713 (Example 11) or vCP1717 (Example 16) or vCP1718 (Example 16) or vCP2017 (Example 18.1) or a mixture of three canarypox viruses vCP1714 (Example 12), vCP1715 (Example 13) and vCP1716 (Example 14) according to the procedure described herein.

### Example 20

### Production of DNA Vaccines for Equines

A DNA solution containing the plasmid pFC104 (Example 6) was concentrated by ethanol precipitation in the manner described by Sambrook et al (1989). The DNA sediment was taken up by a 0.9% NaCl solution so as to obtain a concentration of 1 mg/ml. A 0.75 mM DMREE-DOPE solution is prepared by taking up a DMRIE-DOPE lyophilizate by a suitable sterile H₂O volume.

The formation of plasmid-lipid DNA complexes was brought about by diluting in equal parts the 0.75 mM DMRIE-DOPE solution (1:1) with the 1 mg/ml DNA solution in 0.9% NaCl. The DNA solution was progressively introduced with the aid of a 26 G crimped needle along the wall of the flask containing the cationic lipid solution so as to prevent the formation of foam. Gentle stirring takes place as soon as the two solutions mixed. Finally a composition comprising 0.375 mM of DMRIE-DOPE and 500 μg/ml plasmid was obtained.

It is desirable for all the solutions used to be at ambient temperature for all the operations described herein. DNA/DMRIE-DOPE complexing takes place at ambient temperature for 30 minutes before immunizing the animals.

DNA vaccines can also be produced with DNA solutions containing plasmids pFC104 (Example 6) and pFC106 (Example 8) or containing plasmids pFC105 (Example 7) and pFC106, plasmids pFC115 (Example 17) and pFC106, or containing plasmid pFC101, pFC102 and pFC103 (Examples 3 to 5), or containing plasmid pFC105 or pFC115 according to the procedure described in the present Example.

### Example 21

### In vitro Expression Tests

The expression of WN proteins was tested for each construction by conventional indirect immunofluorescence and Western Blot methods.

These tests were carried out on 96 well plates containing CHO cells cultured in monolayers and transfected by plasmids or containing CEF cells cultured in monolayers and infected by recombinant viruses.

The WN proteins were detected by the use of infected chicken or horse sera and of labelled anti-sera.

The size of the fragments obtained after migration on agarose gel was compared with those expected.

### Example 22

### Effectiveness on Animals

The recombinant vaccines and plasmid vaccines were injected twice at approximately two week intervals into approximately seven day old, unvaccinated SPF chickens by the intramuscular route and in a volume of approximately 0.1 ml. An unvaccinated control group was included in the study.

The chickens were challenged by subcutaneous administration into the neck of 10^{3.5}TCID₅₀ of pathogenic WN virus.

Viremia, antibody response and mortality were observed. Autopsies were carried out to observe lesions.

### Example 23

### Titrating Anti-WNV Neutralizing Antibodies

Dilution series were produced for each serum at a rate of 3 in DMEM medium to which was added 10% fetal calf serum in 96 well plates of the cellular culture type. To 0.05 ml of diluted serum was added 0.05 ml of culture medium containing approximately 100 CCIP₅₀/ml of WNV. This mixture was incubated for 2 hours at 37° C. in an oven in an atmosphere containing 5% CO2.

0.15 ml of a suspension of VERO cells containing approximately 100,000 cells/ml was then added to each mixture. The cytopathic effect (CPE) was observed by phase contrast microscopy after 4 to 5 days culturing at 37° C. in an atmosphere containing 5% CO₂. The neutralizing titers of each serum were calculated using the Karber method. The titers were given in the form of the largest dilution inhibiting the cytopathic effect for 50% of the wells. The titers were expressed in log10 VN50. Each serum was titrated at least twice and preferably four times.

### Example 24

### Test on Horses of vCP2017

Recombinant vaccines containing vCP2017 (Example 18.1) formulated extemporaneously with 1 ml of Carbopol©974P adjuvant (4 mg/ml) were injected twice at 35 day intervals into horses aged more than three months and which had not been previously vaccinated, using the intramuscular route and a volume of approximately 1 ml. Three groups of animals were vaccinated, with doses of 10^{5.8}CCID₅₀ (i.e. 10^{5.64} pfu) for group 1, 10^{6.8}CCID₅₀ (i.e. 10^{6.64} pfu) for group 2 and 10^{7.8}CCID₅₀ (i.e. 10^{7.64} pfU) for group 3. An unvaccinated control group was included in the study.

The neutralizing antibody titers were determined as indicated in Example 23 The titers were expressed in log10 VN50.

| Group | Titers at day 0 | Titers at day 35 | Titers at day 49 |
|---|---|---|---|
| 1 | <0.6 | <0.78 | 2.66 |
| 2 | <0.6 | 1.14 | 2.58 |
| 3 | <0.6 | 1.16 | 2.26 |
| Control | <0.6 | <0.6 | <0.6 |

### Example 25

### Protection After Challenge in Horses

Twenty horses (mares), 3-11 years old, were randomly allocated into two groups of 10 horses each. 1 ml of vaccine containing the recombinant vCP2017 10^{6.3} CCID₅₀ (example 18.1) was formulated extemporaneously with 1 ml of Carbopol© 974P adjuvant at 4 mg/ml. Horses of group 1 were injected twice at 35 day intervals using the intramuscular route and a volume of approximately 1 ml containing 10^{6.0}CCID₅₀ (i.e. 10^{5.84} pfu). One of the vaccinated horses had to be removed from the study prior to challenge due to recurrent colic.

Horses of group 2 remained unvaccinated and served as controls.

Horses from both groups were challenged on day 49 with WNV via the bites from WNV-infected Aedes albopictus mosquitoes. The Aedes albopictus mosquitoes were infected intrathoracically with WNV NY99 eight days prior to the challenge. Each mosquito received approximately 150 pfu. At challenge, a round carton capped with a fine nylon mesh (containing approximately 20 WNV-infected Aedes albopictus mosquitoes) was held (mesh side down) over a clipped area of the horse skin for 5 to 8 minutes.

The neutralizing antibody titers were determined as indicated in example 23. The titers were expressed in log10 VN50.

| Group | D0 | D35 | D42 | D49 | D63 |
|---|---|---|---|---|---|
| Vaccinated | <0.84 | <0.93 | 2.42 | 2.78 | 3.36 |
| Control | <0.72 | <0.75 | <0.78 | <0.78 | 3.43 |

None of the 9 vaccinated horses developed detectable WNV viremia while 8 of 10 control horses developed detectable WNV viremia.

### Example 26

### Test on Cats of vCP2017

41 cats, 14-20 weeks old, were randomly allocated into four groups.

Vaccines containing the recombinant vCP2017 (example 18.1) in 1 ml of sterile water per dose were injected twice at 28 day intervals via the subcutaneous route. Three groups of animals were vaccinated, with doses of 10^{7.9}CCID₅₀ (i.e. 10^{7.74} pfU) for group 1 (8 cats), 10^{6.4}CCID₅₀(i.e. 10^{6.24} pfu) for group 2 (14 cats) and 10^{5.9}CCID₅₀ (i.e. 10^{5.74} pfu) for group 3 (8 cats).

An unvaccinated control group was included in the study. The 11 cats of this group received placebo injections (1.0 ml of Phosphate Buffered saline (PBS) subcutaneously, twice 4 weeks apart).

The neutralizing antibody titers in serum were determined according to example 23. The titers were expressed in log10 VN50.

| Group | D0 | D28 | D42 |
|---|---|---|---|
| Control | <1.01 | <1.01 | <1.03 |
| 1 | <0.9 | <1.08 | 2.26 |
| 2 | <1.08 | <0.99 | 2.16 |
| 3 | <0.95 | <0.97 | 1.36 |

The neutralizing antibody titers in serum were determined with PRNT method (plaque reduction neutralization test; see Bunning M. L. et al., Emerging infectious diseases, 8(4), 380-386, 2002). The titers were expressed as a dilution starting from 1:5. The mean PRNT results at day 42 at 90%, 80% and 50% reduction by group:

| | Titers at day 42 | Titers at day 42 | Titers at day 42 |
|---|---|---|---|
| Group | (reduction of 90%) | (reduction of 80%) | (reduction of 50%) |
| Control | 5.00 | 5.00 | 5.00 |
| 1 | 16.88 | 29.38 | 55.63 |
| 2 | 15.36 | 11.92 | 45.36 |
| 3 | 5.00 | 5.00 | 6.25 |

### Example 27

### Protection After Challenge in Cats

The cats of the groups 2, 3 and control of the example 26 were challenged, 4 months after the second injection (example 26), with WNV via the bites of WNV-infected Aedes albopictus mosquitoes. The Aedes albopictus mosquitoes were infected intrathoracically with WNV NY99 8-10 days prior to the challenge. Each mosquito received approximately 150 pfu. At challenge, a round carton capped with a fine nylon mesh (containing approximately 5-15 WNV-infected Aedes albopictus mosquitoes) was held (mesh side down) over a clipped area of the cat skin for 5 to 10 minutes. The feeding of mosquitoes was confirmed by visualization of engorgement.

A representative sample of engorged, infected mosquitoes was titrated for WNV in order to determine the infection rate of mosquitoes. About 3 representative engorged mosquitoes from each cat were titrated for WNV. The results are 8.4 log pfu/mosquito for control group cats, 8.4 log pfu/mosquito for group 2 cats and 8.3 log pfu/mosquito for group 3 cats.

The neutralizing antibody titers and post-challenge WNV viremia were determined. The titers were calculated with PRNT method (plaque reduction neutralization test) and expressed in dilution starting at 1:5.

The mean PRNT results at 90% reduction by group and by post-challenge day:

| Group | D0 | D7 | D14 |
|---|---|---|---|
| Control | 5.00 | 5.00 | 20 |
| 2 | 7.14 | 89.64 | 148.57 |
| 3 | 5.00 | 47.50 | 47.50 |

The mean PRNT results at 80% reduction by group and by post-challenge day:

| Group | D0 | D7 | D14 |
|---|---|---|---|
| Control | 5.00 | 5.45 | 21.82 |
| 2 | 9.29 | 101.43 | 214.29 |
| 3 | 5.63 | 53.75 | 125.00 |

Incidence of WN virus isolation (number of cats having a positive WN virus isolation) by group and by post-challenge day:

| Group | Day 0 | day 1 | day 2 | day 3 | day 4 | day 5 | day 6 | days 7-10 | day 14 |
|---|---|---|---|---|---|---|---|---|---|
| Control | 0 | 0 | 3 | 5 | 7 | 7 | 5 | 0 | 0 |
| 2 | 0 | 0 | 0 | 0 | 0 | 0 | 0 | 0 | 0 |
| 3 | 0 | 0 | 1 | 0 | 0 | 0 | 0 | 0 | 0 |

None of the 14 vaccinated cats of group 2 developed a detectable WNV viremia, only one of the 8 vaccinated cats of group 3 developed a detectable WNV viremia while 9 of 11 control cats developed a detectable WNV viremia.

### Example 28

### Construction of the Recombinant Viruses vFP2000

As illustrative of one embodiment of the invention a specific fowlpox recombinant construct is described in this Example.

A PCR reaction was performed on the basis of the matrix constituted by genomic DNA of a fowlpox virus (DIFTOSEC CT(D strain marketed by MERIAL) and with the following oligonucleotides:

| | |
|---|---|
| F8FCA1 (42 mer) (SEQ ID NO: 30): | 5′ATCATCGAGCTCGACCCTTTACAAGAATAAAAGAAGAAACAA3′ |
| and F8FCB1 (73 mer) (SEQ ID NO: 31): | 5′CTCGAGCTGCAGGAATTCCCCGGGTTTTTATTAGCTAATTAGCAATATAGATTCAA |
| | TATGATAATTACTCTAA3′ |

in order to isolate a 1483 bp PCR fragment (fragment H).

A PCR reaction was carried out on the basis of the matrix constituted by genomic DNA of the fowlpox virus and with the following oligonucleotides:

| | |
|---|---|
| F8FCC1 (72 mer) (SEQ ID NO: 32): | 5′CCCGGGGAATTCCTGCAGCTCGAGTTTTTATTGACTAGTTAATCATAAGATAAATA |
| | ATATACAGCATTGTAA3′ |
| and F8FCD1 (45 mer) (SEQ ID NO: 33): | 5′GATGATGGTACCGGGTAATGGCTTTTGTTTATAACCACGTTTGTC3′ |

in order to isolate a 1433 bp PCR fragment (fragment I).

Fragments H and I were hybridized together in order to serve as a matrix for a PCR reaction performed with the oligonucleotides F8FCA1 (SEQ ID NO: 30) and F8FCD1 (SEQ ID NO: 33) in order to generate a 2892 bp PCR fragment. This fragment was digested by the restriction enzymes Sacd and KpnI in order to isolate, following agarose gel electrophoresis, a 2875 bp SacI-KpnI fragment. This fragment was ligated with the pBlueScript® II SK+vector (Stratagene, La Jolla, USA, Cat # 212205), previously digested by the restriction enzymes Sacd and KpnI, in order to give the plasmid pF8L. The sequence of this plasmid was verified by sequencing. This plasmid contains 1424 bp of sequences upstream of ORF F8 (left flanking arm F8), an early transcription stop vaccine signal, stop codons in 6 reading frames, a multiple cloning site containing restriction sites Smal, PstI, XhoI and EcoRi and finally 1376 bp of sequences located downstream of ORF F8 (right flanking arm F8).

The plasmid pMP528HRH (Perkus M. et al. J. Virol. 1989, 63, 3829-3836) was used as the matrix for amplifying the complete sequence of the vaccine promoter H6 (GenBank accession no. M28351) with the following oligonucleotides:

| | |
|---|---|
| FC125 (95 mer) (SEQ ID NO: 34) | 5′AAACCCGGGTTAATTAATTAGTCATCAGGCAGGGCGAAACGAGACTATCTGCTCG |
| | TTAATTAATTAGAGCTTCTTTATTCTATACTTAAAAAGTG3′ |
| and FC126 (43 mer) (SEQ ID NO: 35) | 5′AAAACTGCAGGTCGACTACGATACAAACTTAACGGATATCGCG3′ |

in order to amplify a 211 bp PCR fragment. This fragment was digested by restriction enzymes Smal and PstI in order to isolate, following agarose gel electrophoresis, a 200 bp SmaI-PstI restriction fragment. This fragment was then ligated with the plasmid pF8L, previously digested by Smal and PstI, in order to give the plasmid pFC 121.

A PCR reaction was performed using the plasmid pFC115 (example 17) as the matrix and the following oligonucleotides:

FC127 (58 mer) (SEQ ID NO: 36)
5′ TTTTCGCGATATCCGTTAAGTTTGTATCGTAATGACCGGAATTGCAG
TCATGATTGGC 3′
and
FC128 (43 mer) (SEQ ID NO: 37)
5′ TTTTGTCGACTCTAGATAAAAATTAAGCGTGCACGTTCACGGA 3′

in order to amplify an approximately 2111 bp PCR fragment. This fragment was digested by restriction enzymes NruI and SalI in order to isolate, following agarose gel electrophoresis, an approximately 2096 bp NruI-SalI restriction fragment. This fragment was then ligated with plasmid pFC121 previously digested by restriction enzymes NruI and XhoI in order to give the plasmid pFC122.

Plasmid pFC122 was linearized by PvuI, then transfected in primary chicken embryo cells infected with the fowlpox virus according to the previously described calcium phosphate precipitation method (Panicali et Paoletti Proc. Nat. Acad. Sci. 1982, 79, 4927-4931; Piccini et al. In Methods in Enzymology, 1987, 153, 545-563, publishers Wu R. and Grossman L. Academic Press). Positive plaques were selected on the basis of a hybridization with a radioactively labelled probe specific to the nucleotide sequence of the envelope glycoprotein E. These plaques underwent 4 successive selection/purification cycles until a pure population was isolated. A representative plaque corresponding to in vitro recombination between the donor plasmid pFC122 and the genome of the fowlpox virus was then amplified and the recombinant virus stock obtained was designated vFP2000, fowlpox WNV prM-M-E.

### Example 28.1

### Construction of a pF8 H6p WNV prM-M-E donor plasmid (pSL-5513-1-1-1) for the Generation of Fowlpox WNV (vFP2000)

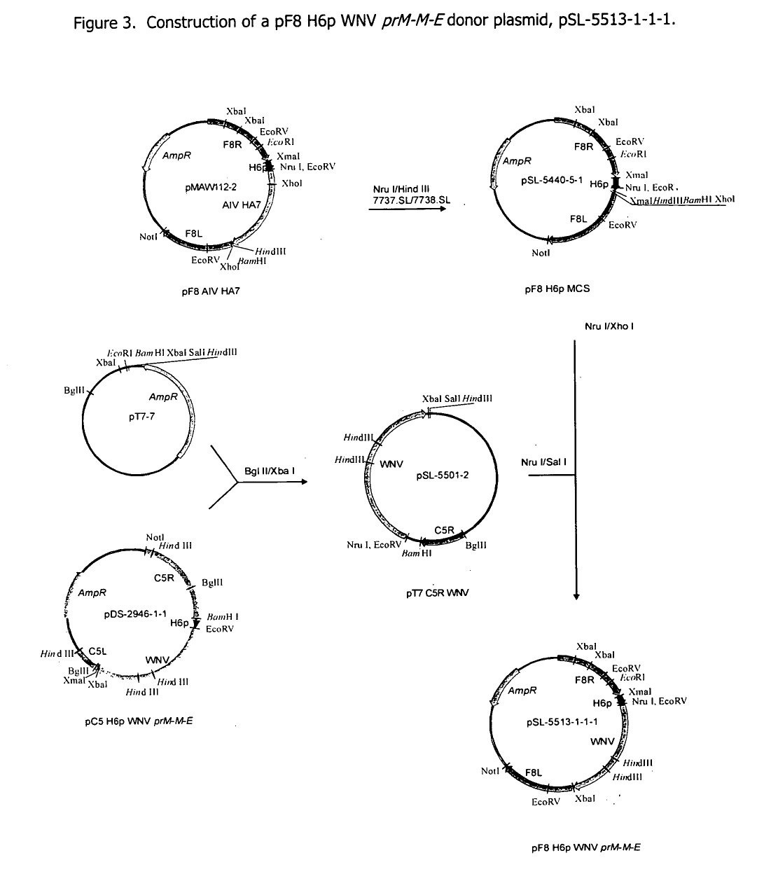

The construction scheme is illustrated in FIG. 3.

There is one commonly used recombination site in the Fowlpox genome, designated as F8. Plasmid pMAW112-2/F8 AIV HA has been described in patent application xxx and was used as the source of the Fowlpox F8 arms. The 1.7 kb AIV HA insert in pMAW112-2/F8 AIV HA was deleted by digestion with Nru I and Hind III, to be replaced by oligonucleotides 7737.SL (SEQ ID NO: ) and 7738.SL (SEQ ID NO: ) encoding the 3′-end of the H6 promoter and a Sma I/Xma I site. The correct insertion of the oligos was confirmed in plasmid pF8 H6p MCS, pSL-5440-5-1. This plasmid contains ˜1.4 kb of the upstream F8 flanking sequence, designated as F8R and ˜1.4 kb of the downstream flanking sequence of F8, designated as F8L, as well as the H6 promoter and a multiple cloning site of Xma I, Hind III, BamH I and Xho I.

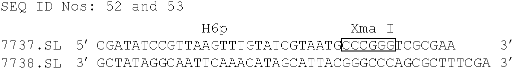

Plasmid pDS-2946-1-1 is an ALVAC donor plasmid that contains the H6 promoter and West Nile Virus prM-M-E genes, between the C5 arms and is described in Example 18.1 and FIG. 1. pDS-2946-1-1 was digested with Bgl II and Xba I and the 2.8 kb C5R-H6p WNV fragment was inserted into pT7-7, between Bgl II and Xba I, generating plasmid pT7C5R-WNV, pSL-5501-2. Plasmid pSL-5501-2 was digested with Nru I and SalI and the 2.1 kb fragment inserted into pSL-5440-5-1 that had been digested with Nru I and Xho. The resultant pF8 H6p WNV prM-M-E plasmid, pSL-5513-1-1-1, was confirmed by restriction enzyme digestion and nucleotide sequence analysis. The sequence of the F8-H6p WNV prM-M-E-F8 gene cassette from pSL-5513-1-1-1 is shown in FIG. 4.

### Example 28.2

### Generation and Characterization of a Fowlpox Recombinant Expressing West Nile Virus prM-M-E, vFP2000.

Primary CEFs were transfected with Not I-linearized pSL-5513-1-1-1 plasmid DNA (15 ug) using Fugene reagent (Roche) by the method suggested by the supplier. The transfected cells were subsequently infected with fowlpox as rescue virus at MOI of 10 and after ˜24 h, the transfected-infected cells were harvested, sonicated and used for recombinant virus screening. Recombinant plaques were screened based on the plaque lift hybridization method using a WNV-specific probe, which was directly labelled with horseradish peroxidase according to the manufacturer's protocol (Amersham). After five sequential rounds of plaque purification, the recombinants designated as vFP2000.2.1.1.1.1 and vFP2000.3.2.1.2.1 were generated and confirmed by hybridization as 100% positive for the WNV insert and 100% negative for the F8 ORF. Agarose punches were selected from the fifth round of plaque purification, and expanded to obtain stocks of vFP2000. Recombinant viruses were characterized by restriction enzyme and Southern blot analyses. The F8-H6p WNV-F8 locus was PCR-amplified and the complete sequence confirmed. Expression of the WNV proteins was confirmed by immunoplaque and immunoblot analyses. The fowlpox recombinants contain one copy of the H6p WNV prM-M-E gene cassette.

### Example 29

### Test on Geese of vFP2000

20 Chinese geese, one-weeks old, were randomly allocated into four groups.

Vaccine was prepared by mixing extemporaneously 1 ml of the recombinant vFP2000 (Example 28) at 10^{6.3} CCID₅₀ with 1 ml of Carbopol© 974P adjuvant at 4 mg/ml. One group of 5 birds was vaccinated with 0.2 ml by the intramuscular route twice at 13 day interval (called vFP2000 group).

Two unvaccinated control groups were included in the study. One control group of 7 geese was not vaccinated and was not challenged (called sham control group). One control group of 8 geese was not vaccinated but was challenged (called challenged control group). The geese of these groups received by intramuscular route placebo injections (0.2 ml of a Carbopol© 974P solution at 2 mg/ml) twice 13 days apart.

Geese from the vFP2000 group and from the control group were challenged on day 26 with 0.2 ml containing about 10^{3.5} CCID₅₀ of WNV by subcutaneous route.

The morbidity and post-challenge WNV viremia were observed. The virus titer was calculated and expressed as TCID₅₀/0.1 ml virus titers.

| Group | D0 | D1 | D2 | D3 | D4 | D5 | D7 | D10 |
|---|---|---|---|---|---|---|---|---|
| Challenged | <0.3 | 4.2 | 4.5 | 3.3 | 3.9 | 0.7 | <0.3 | <0.3 |
| control | | | | | | | | |
| vFP2000 | <0.3 | 0.5 | 0.6 | 0.5 | 0.8 | <0.3 | <0.3 | <0.3 |
| Sham | <0.3 | <0.3 | <0.3 | <0.3 | <0.3 | <0.3 | <0.3 | <0.3 |
| control | | | | | | | | |

The viremia was expressed as the percentage of WNV-excreting animals for each group.

| Group | D0 | D1 | D2 | D3 | D4 | D5 | D7 | D10 |
|---|---|---|---|---|---|---|---|---|
| Challenged | 0% | 87.5% | 100% | 100% | 100% | 62.5% | 0% | 0% |
| control | | | | | | | | |
| vFP2000 | 0% | 20% | 40% | 20% | 40% | 0% | 0% | 0% |
| Sham | 0% | 0% | 0% | 0% | 0% | 0% | 0% | 0% |
| control | | | | | | | | |

None of the five vFP2000 vaccinated geese developed detectable morbidity while 4 of 8 challenged control geese developed detectable morbidity. None of the sham control geese developed detectable morbidity.

### Example 30

### Immunoblot Analysis of the Expression of WNV Proteins from ALVAC WNV (vCP2017), ALVAC-2 WNV (vCP2018) and Fowlpox WNV (vFP2000) Recombinants

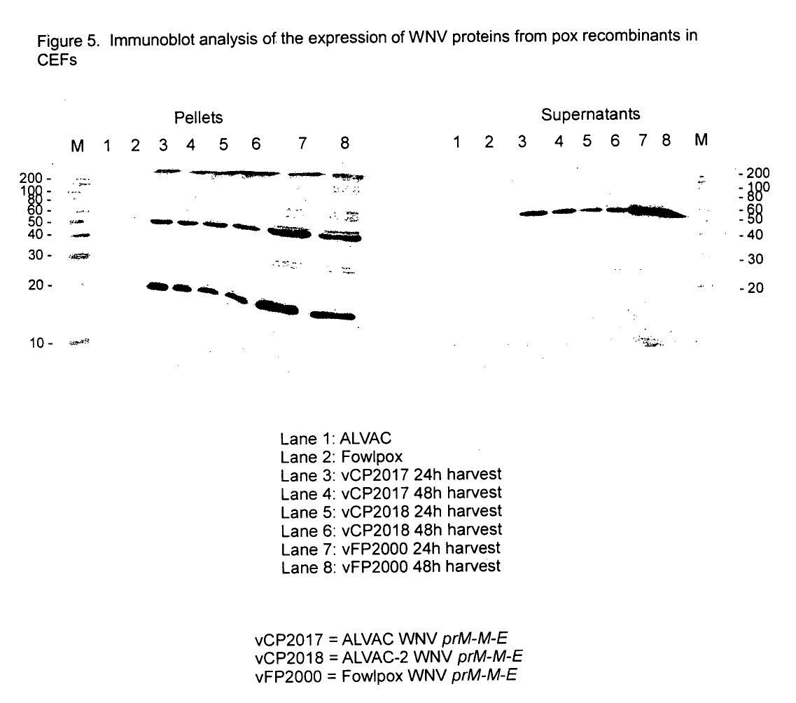

ALVAC recombinants vCP2017 and vCP2018 each contain two copies of the H6p WNV prM-M-E gene cassette located at the C5 loci, while vFP2000 contains a single copy of H6p WNV prM-M-E at the F8 locus. CEFs were infected with vCP2017, vCP2018 or vFP2000 at an MOI of 10 and grown in serum-free DMEM medium for 24 and 48 hours at 37° C., 5% CO₂. The culture medium was collected and the cells were resuspended in PBS, then centrifuged and the PBS discarded. Cell lysate samples were prepared by resuspending the pellets in water, adding 5×SDS-PAGE sample buffer (without β-mercaptoethanol), and boiling for 5 minutes. Supernatant samples were prepared by adding 5×SDS-PAGE sample buffer (without β-mercaptoethanol), and boiling for 5 minutes. Samples were run on a 12% SDS-PAGE gel and the separated proteins were transferred to nylon membrane (Millipore Immobilon P). The immunoblot was probed with chicken anti-WNV antisera (Merial Ltd.), then horseradish peroxidase conjugated donkey-anti-chicken antibody (Jackson Labs) was used as secondary antibody. The reactive bands were visualized with Chemiluminescence Reagent (Perkin Elmer). As illustrated in FIG. 5, the expression of both the WNV E and M proteins was higher in the cell fraction from the fowlpox recombinant than from the ALVAC recombinants. The production of the WNV E protein was also higher in the supernatant fraction from the fowlpox recombinant than the ALVAC recombinants, but there was no M protein secreted.

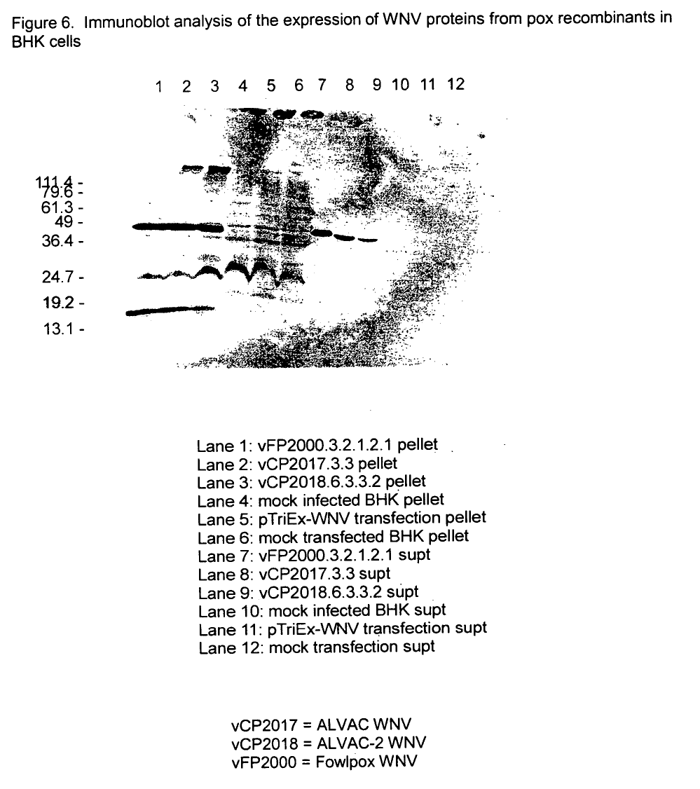

In order to determine whether this finding was reproducible in non-avian cells, baby hamster kidney (BHK) cells were infected with vCP2017, vCP2018 or vFP2000 at an MOI of 10. After ˜24 h, culture supernatants and cells were collected, as described above. For SDS-PAGE analysis, 5×sample buffer (without β-mercaptoethanol) was added to the culture supernatants and the samples boiled for 5 min. The infected cells were resuspended in PBS, then spun to concentrate them and for SDS-PAGE analysis, lysis buffer was added, samples boiled for 5 min, then 5×sample buffer was added and the samples re-boiled for 5 min. Samples were separated on a 12% SDS-PAGE gel and proteins transferred to Millipore Immobilon P nylon membrane. Chicken anti-WNV antibody (Merial Ltd.) was used as primary antibody and horseradish peroxidase-conjugated donkey-anti-chicken antibody (Jackson Labs) was used as secondary antibody. Reactive protein bands were visualized with Chemiluminescence Reagent (Perkin Elmer). As illustrated in FIG. 6, the expression of the WNV E protein in the supernatant fraction from the fowlpox recombinant was higher than the ALVAC recombinants, in mammalian cells. There was no obvious difference in expression levels for the various recombinants in the cell fraction, however the fowlpox recombinant has only one gene copy compared to two gene copies in the ALVAC vectors.

### Example 31

### Generation and Characterization of Specific Antisera to West Nile Virus Proteins Expressed by ALVAC WNV (vCP2017), ALVAC-2 WNV (vCP2018) and Fowlpox WNV (vFP2000) Recombinants

The in vitro expression of West Nile Virus M and E proteins from ALVAC or fowlpox recombinants in primary CEFs and BHK cells was demonstrated in Example 30. In order to determine whether the recombinants were immunogenic and if there was a demonstrable difference in the quality of the antisera, mice were immunized with vCP2017, vCP2018 and vFP2000. Balb/c mice (Charles River, Quebec) were immunized IM with 4×10⁷ pfu of recombinant virus in 1 mM PBS, at days 0 and 21. Blood from day 35 was processed and used to probe immunoblots. Samples for immunoblot analysis of antisera from ALVAC WNV immunizations were prepared by transient transfection of Chinese hamster ovary (CHO) cells with pTriEx-WNV DNA, which had been shown to express the WNV M and E genes. CHO cells were transfected with 10ug of pTriEx-WNV DNA, using electroporation at settings 1.5 kV, 25 uF and infinite resistance. Mock samples were prepared by the electroporation of CHO cells at the same settings, without DNA. After approximately 65 hours, supernatants were harvested and clarified by spinning at 3000 rpm for 5 min. The plates were washed twice with PBS, then 500 μl of PBS was added and the cells were scraped off. After spinning at 3000 rpm for 5 min, the supernatant was removed and the cells resuspended in 100 μl of SDS-PAGE lysis buffer. Samples were suspended in SDS-PAGE loading buffer (minus β-mercaptoethanol) and separated on a 12% SDS-PAGE gel before electrotransfer to Immobilon P nylon membrane. The membranes were processed and probed with 1:1000 dilution of the mouse anti-WNV antisera. Peroxidase-conjugated goat anti-mouse antisera (Jackson Labs) were used as secondary antibody at a dilution of 1:500 and bands were visualized using Chemiluminescence Reagent (Perkin Elmer). Five of five mouse anti-WNV antisera generated by vCP2017 immunization, reacted specifically with a protein band in the pTriEx-WNV samples, of the expected size for the WNV E protein. All of these antisera also reacted with media components in both the pTriEx-WNV and mock samples. Four of five mouse anti-WNV antisera generated by vCP2018 immunization, reacted specifically with a protein band in the pTriEx-WNV samples, of the expected size for the WNV E protein. All of the five antisera also reacted with media components in both the pTriEx-WNV and mock samples.

For analysis of anti-WNV antibodies following vFP2000 immunization, vFP2000 pellet and supernatant samples were run on 12% SDS-PAGE gels and transferred to Immobilon P nylon membrane (Millipore). The membranes were processed and probed with 1:1000 dilution of the mouse anti-WNV antisera. Peroxidase-conjugated goat anti-mouse antibody (Jackson Labs) was used as secondary antibody at a dilution of 1:500 and bands were visualized using Chemiluminescence Reagent (Perkin Elmer). Five of five mouse anti-WNV antisera generated by vFP2000 immunization, recognized specific bands in the vFP2000 samples, corresponding to WNV E and M proteins. Anti-vFP2000 antisera also recognized specific WNV bands expressed in vCP2017 and vCP2018.

Having thus described in detail preferred embodiments of the present invention, it is to be understood that the invention defined by the appended claims is not to be limited to particular details set forth in the above description as many apparent variations thereof are possible without departing from the spirit or scope of the present invention.

1. An immunogenic composition to induce an immune response against West Nile virus (WNV) in an animal susceptible to WNV comprising a vector comprising a recombinant virus or DNA plasmid that encodes and expresses in vivo in the animal WNV E; WNV prM and E; WNV M and E; WNV prM, WNV M and E, WNV polyprotein prM-E, WNV polyprotein M-E, or WNV polyprotein prM-M-E.

2. The immunogenic composition of claim 1 wherein the recombinant virus is a recombinant adenovirus, herpesvirus or poxvirus.

3. The immunogenic composition of claim 2 wherein the recombinant virus is a recombinant poxvirus.

4. The immunogenic composition of claim 3 wherein the recombinant poxvirus is a recombinant avipox virus.

5. The immunogenic composition of claim 4 wherein the recombinant avipox virus is a cannarypox or fowlpox virus.

6. The immunogenic composition of claim 5 wherein the canarypox virus is ALVAC and the fowlpox virus is TROVAC.

7. The immunogenic composition of claim 1 wherein the nucleic acid molecule is a coding frame encoding polyprotein prM-M-E.

8. The immunogenic composition of claim 5 wherein the nucleic acid molecule is a coding frame encoding polyprotein prM-M-E

9. The immunogenic composition of claim 6 wherein the nucleic acid molecule is a coding frame encoding polyprotein prM-M-E

10. The immunogenic composition of claim 1 wherein the nucleic acid molecule comprises nucleotides 466-741, 742-966 and 967-2469 of GenBank AF196835 encoding WNV prM, M and E, respectively.

11. The immunogenic composition of claim 1 wherein the nucleic acid molecule comprises nucleotides 466-2469 of GenBank AF196835 (SEQ ID NO: 66) encoding WN protein prM-M-E.

12. The immunogenic composition of claim 1 wherein the nucleic acid molecule comprises nucleotides 421-2469 of GenBank AF196835 (SEQ ID NO: 66) encoding WN protein prM-M-E and the signal peptide of prM.

13. The immunogenic composition of claim 1, further comprising an adjuvant.

14. The immunogenic composition according to claim 10, wherein the adjuvant is a carbomer.

15. The immunogenic composition of claim 1 further comprising an antigen or immunogen or epitope thereof of a pathogen other than WNV of the animal, or a vector that contains and expresses in vivo in the animal a nucleic acid molecule encoding the antigen, immunogen or epitope thereof, or an inactivated or attenuated pathogen other than WNV of the animal.

16. The immunogenic composition of claim 1, wherein the animal is a cat or a horse.

17. A method for inducing an immunological or protective immune response against WNV in an animal comprising administering to the animal the immunogenic or vaccine composition according to claim 1.

18. A method for inducing an immunological response against WNV in an animal comprising administering to the animal the immunogenic or vaccine composition according to claim 17.

19. The method according to claim 18 wherein the adjuvant comprises a carbomer adjuvant.

20. A method for inducing an immunological response against WNV in an animal and against another pathogen of the animal comprising administering to the animal the immunogenic composition according to 19.

21. A method for inducing an immunological response against WNV in an animal comprising administering to the animal (a) the immunogenic composition according to claim 1, and (b) a WNV isolated antigen, immunogen or epitope thereof, wherein (a) is administered prior to (b) in a prime-boost regimen, or (b) is administered prior to (a) in a prime-boost regimen, or (a) and (b) are administered together, either sequentially or in admixture.

22. The method of any of claims 17, 20 or 21, wherein the animal is a cat or a horse.

23. A differential diagnosis method comprising administering to animals an immunogenic composition of claim 1, and/or a WNV antigen, immunogen or epitope, and testing the animals for presence or absence of a WNV protein or antibody thereto not expressed by the immunogenic or vaccine composition or not administered as the WNV antigen, immunogen or epitope.

24. The method of claim 23, wherein the animal is a cat or a horse.

25. A kit for performing the method of claim 23 comprising (a) and (b) in separate containers, optionally with instructions for admixture and/or administration.

26. A kit for performing the method of claim 24 comprising the immunogenic composition and/or the WNV antigen, immunogen or epitope, and an assay for testing for the presence or absence of the WNV protein, in separate containers, optionally with instructions for administration of the immunogenic or vaccine composition and/or the WNV antigen, immunogen or epitope, and/or for performing the assay.

27. A kit comprising (a) the immunogenic composition according to claim 1, and (b) the antigen or immunogen or epitope thereof of a pathogen other than WNV of the animal, or the vector that contains and expresses in vivo in the animal a nucleic acid molecule encoding the antigen, immunogen or epitope thereof, or the inactivated or attenuated pathogen other than WNV of the animal, wherein (a) and (b) are in separate containers, and the kit optionally contains instructions for admixture and/or administration of (a) and (b).

28. The kit of claim 25 or 27, wherein the animal is a cat or a horse.

29. A plasmid that encodes and expresses in vivo in an animal susceptible to West Nile Virus WNV E; WNV prM and E; WNV M and E; WNV prM, WNV M and E, WNV polyprotein prM-E, WNV polyprotein M-E, or WNV polyprotein prM-M-E.
